(19) United States
(12) Patent Application Publication
(10) Pub. No.: US 2024/0287596 A1
(43) Pub. Date: Aug. 29, 2024

(54) METHOD FOR SEQUENCING RNA OLIGONUCLEOTIDES

(71) Applicant: CeMM - Forschungszentrum für Molekulare Medizin GmbH, Wien (AT)

(72) Inventors: Paul DATLINGER, Wien (AT); Christoph BOCK, Wien (AT)

(73) Assignee: CeMM - Forschungszentrum für Molekulare Medizin GmbH, Wien (AT)

(21) Appl. No.: 17/641,048

(22) PCT Filed: Sep. 7, 2020
(86) PCT No.: PCT/EP2020/074985

§ 371 Date: Sep. 8, 2022

(30) Foreign Application Priority Data
Sep. 6, 2019 (EP) 19196008.7
Dec. 16, 2019 (EP) 19216696.5

(51) Int. Cl. C12Q 1/6869

(52) U.S. Cl. CPC C12Q 1/6869

(57) ABSTRACT

The invention relates to a method for sequencing oligonucleotides comprising RNA, wherein two indexing sequences are introduced in RNA oligonucleotides. The invention furthermore relates to uses of such methods and devices used for such methods. Further provided are kits comprising one or more components used in the methods of the invention.

The present application is a national phase application under 35 U.S.C. § 371 of International Application No. PCT/EP2020/074985, filed Sep. 7, 2020, which claims the priority benefit of European Application No. 19216696.5, filed Dec. 16, 2019, and European Application No. 19196008.7, filed Sep. 6, 2019, the entire contents of each of which are hereby incorporated by reference.

This application contains a Sequence Listing which has been submitted electronically in ASCII format and is hereby incorporated by reference in its entirety. The ASCII copy, created on Aug. 30, 2022, is named VOSSP0127US_ST25_2.txt and is 5,658 bytes in size.

The invention relates to a method for sequencing oligonucleotides comprising RNA, the method comprising the steps of (a) providing permeabilized cells and/or nuclei comprising a first oligonucleotide comprising RNA; (b) combining said cells and/or nuclei of (a) with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide; (c) reversely transcribing the first oligonucleotide in said cells/nuclei to obtain an elongated second oligonucleotide; (d) combining said cells and/or nuclei obtained in step (c) with a microbead-bound third oligonucleotide in a second reaction compartment, wherein the third oligonucleotide comprises (i) a first sequence corresponding to a fourth sequence comprised in the second oligonucleotide used in step (b); or (ii) a first sequence complementary to a first sequence of a fourth oligonucleotide, wherein the fourth oligonucleotide further comprises a second sequence at least partially complementary to the third sequence of the second oligonucleotide; wherein for (i), the method further comprises a step of second strand DNA synthesis subsequent to step (c) and prior to step (d) and wherein for (ii) the method further comprises a step of DNA ligation; and wherein the third oligonucleotide further comprises a second sequence comprising an indexing sequence and a third sequence comprising a primer binding site; (e) amplifying the DNA oligonucleotides obtained in step (d); and (f) sequencing of amplified DNA oligonucleotides. The invention furthermore relates to uses of such methods and devices used for such methods. Further provided are kits comprising one or more components used in the methods of the invention.

Cell atlas projects (e.g., the Human Cell Atlas (Rozenblatt-Rosen et al. (2017) Nature 550, 451-3) and single-cell CRISPR screens (e.g. using CROP-seq (Datlinger et al. (2017) Nat Methods 14, 297-301)) hit the limits of current technology, as they require profiling of millions of single cells. Most single-cell RNA-seq studies that reach beyond the scale of what is feasible using standard microtiter (96-well or 384-well) plates are currently based on either sub-nanoliter well plates or on microfluidic droplet generators. Both technologies build on a micro-manufacturing method called soft lithography.

In sub-nanoliter well-based scRNA-seq (Cyto-Seq (Chen et al. (2015) Science 348, aaa6090), Seq-Well (Gierahn et al. (2017) Nat Methods 14, 395-8), Microwell-Seq (Han et al. (2018) Cell 172, 1091-1107), sci-RNA-seq (Cao et al. (2017) Science 357, 661-7)), a plate with miniaturized reaction compartments in the sub-nanoliter range is cast from a material such as PDMS or agarose. Beads and cells are loaded by gravity. While beads are typically loaded to near saturation, cells are loaded at a limiting dilution (i.e., very low concentration) to avoid cells entering the same reaction compartment. If two cells did enter the same well on the plate, they would end up with the exact same cell barcode and would be indistinguishable in the downstream analysis. On the plate, cells are lysed and their transcriptome anneals to complementary oligonucleotides on the microbeads. Typically, beads are then collected, and the reverse transcription is performed in bulk. Currently, there is a lack of well-validated and readily available protocols and commercial solutions, so that most labs prefer microfluidic droplet generators (described next).

Soft lithography is not limited to open designs such as sub-nanoliter well plates. When using PDMS as the material, the open side can be sealed by bonding it to a glass slide to realize complex channel designs. This has allowed the manufacturing of microfluidic droplet generators for scRNA-seq (Drop-seq (Macosko et al. (2015) Cell 161, 1202-14), inDrop (Klein et al. (2015) Cell 161, 1187-1201), 10× Genomics Chromium (Zheng et al. (2017) Nat. Commun. 8, 14049)). A typical microfluidic device for scRNA-seq has four inputs (for cells, barcoded microbeads, reverse transcription reagents, and carrier oil) and one output (for the droplet emulsion). The reverse transcription reaction is typically performed inside the droplets. While deformable beads can be loaded to near saturation, cells are supplied at a limiting dilution to make it unlikely that two cells enter the same droplet. If two cells did enter the same droplet, they would receive the exact same cell barcode and would be indistinguishable in the downstream analysis. As a consequence, while most droplets contain both reagents and beads and are thus fully functional, they are ultimately not used because they do not contain a cell.

The throughput of sub-nanoliter well plates and microfluidic droplet generators is limited by the requirement to load cells at a limiting dilution to avoid cell doublets. These platforms typically reach a throughput of about 10,000 cells per experiment (e.g. per sub-nanoliter well plate or per channel on the 10× Genomics Chromium chip) but this can be increased by parallelization (multiple plates, multiple channels on the microfluidic device). However, this often comes at high cost and is labour-intensive.

In combinatorial indexing, the number of cells profiled can scale exponentially with the number of barcoding rounds. Two rounds of barcoding allow the profiling of roughly 10,000 cells (when using 384×384 barcodes), which generates a lot of manual work, but does not provide any advantage over sub-nanoliter well plates or droplet generators. Only when a third round of indexing is introduced, the processing of over one million cells becomes possible. The currently largest dataset generated with sci-RNA-seq v3 comprises 2 million single-cell transcriptomes from the developing mouse embryo (Cao et al. (2019) Nature 566, 496-502). However, this comes with several drawbacks: (1) most NGS library preparation protocols are not immediately compatible with three rounds of combinatorial indexing (e.g. assays such as ATAC-seq, DNA methylation profiling, Hi-C). (2) In each barcoding step, nuclei or cells have to remain intact despite aggressive reaction buffers and high temperature incubations. With three barcoding rounds, the loss of material is typically >90%. (3) It is challenging to design an elegant library read structure to sequence the combination of three barcodes cost effectively (this is particularly problematic when ligation overhangs have to be sequenced along with the barcodes such as in SPLIT-seq or sci-RNA-seq v3. (4) Synthesis and sequencing errors in the barcodes accumulate, so that a larger percentage of reads cannot be assigned with confidence. (5) Running reactions on intact cells or nuclei is only partially efficient. The more reactions have to be run this way, the lower the overall efficiency of the library preparation and quality of the resulting single-cell transcriptomes. (6) To achieve high cell numbers, a large number of indices have to be used for each barcoding round. As an example, to generate the 2 million cell dataset a combination of 384×384×768 barcodes was used. This is both labor-intensive and wasteful in terms of the reagent volumes required. Given these disadvantages, it is hard to imagine that published methods for combinatorial indexing scRNA-seq will be universally adopted by research labs or become a commercial success.

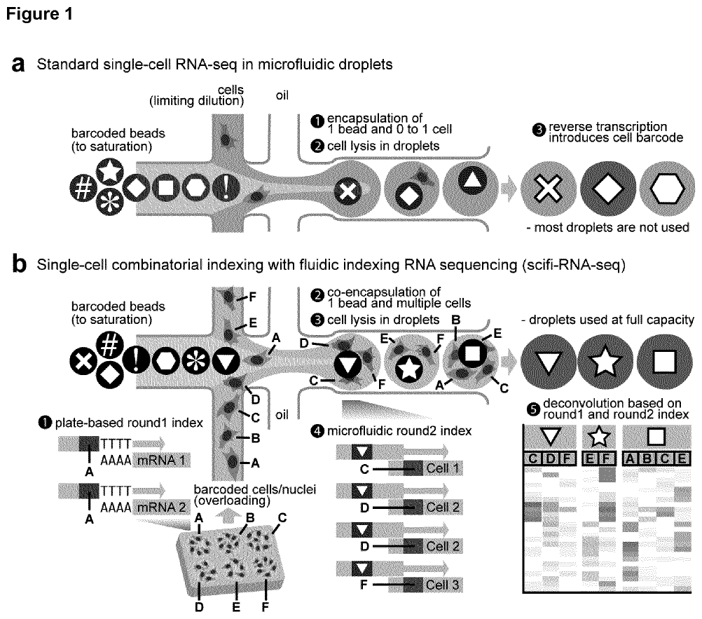

In a typical experiment, the cell suspension is loaded onto a microfluidic chip, along with a population of microbeads with unique DNA barcodes, reverse transcription reagents, and carrier oil (FIG. 1a). When aqueous and oil phases are combined at controlled flow rates, emulsion droplets co-encapsulate individual cells with individual microbeads. Due to the buffer composition, cells are lysed, and cellular macromolecules are released into the droplet. Cellular transcripts anneal to complementary, bead-tethered primers carrying a unique cell barcode. For whole transcriptome applications these primers contain an oligo-dT stretch complementary to the poly-A tail in messenger RNAs. But in principle, any capture sequence can be used so that specific transcripts or RNAs can be selectively enriched. In some implementations, the microbead is dissolved by reducing conditions or UV light for a more efficient transcript capture. In most protocols, the emulsion droplets are used as reaction compartments for the reverse transcription reaction, which incorporates the barcode into the cell's transcriptome.

Importantly, if two cells enter the same droplet or the same well, e.g. on a sub-nanoliter well plate, their transcriptomes are labelled with the exact same cell barcode, resulting in a cell doublet that confounds the analysis. To avoid this issue, state-of-the-art droplet generators are supplied with the cell suspension at a limiting dilution, with most droplets carrying 0 or 1 cells. This makes microfluidic scRNA-seq highly inefficient. While most emulsion droplets are fully functional (they contain both barcoded microbeads and reverse transcription reagents), they do not receive a cell and thus do not result in a productive library preparation event.

Accordingly, there is a need for improved methods for analyzing RNA oligonucleotides, in particular methods allowing high throughput analysis.

The technical problem is solved by the embodiments provided herein and in particular as provided in the claims.

The present invention relates to, inter alia, the following items:

1. A method for sequencing oligonucleotides comprising RNA, the method comprising the steps of:
(a) providing permeabilized cells and/or nuclei comprising a first oligonucleotide comprising RNA;
(b) combining said cells and/or nuclei of (a) with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide;
(c) reversely transcribing the first oligonucleotide in said cells/nuclei to obtain an elongated second oligonucleotide;
(d) combining said cells and/or nuclei obtained in step (c) with a microbead-bound third oligonucleotide in a second reaction compartment, wherein the third oligonucleotide comprises
   (i) a first sequence corresponding to a fourth sequence comprised in the second oligonucleotide used in step (b); or
   (ii) a first sequence complementary to a first sequence of a fourth oligonucleotide, wherein the fourth oligonucleotide further comprises a second sequence at least partially complementary to the third sequence of the second oligonucleotide;
   wherein for (i), the method further comprises a step of second strand DNA synthesis subsequent to step (c) and prior to step (d) and wherein for (ii) the method further comprises a step of DNA ligation;
   and wherein the third oligonucleotide further comprises a second sequence comprising an indexing sequence and a third sequence comprising a primer binding site;
(e) amplifying the DNA oligonucleotides obtained in step (d); and
(f) sequencing of amplified DNA oligonucleotides.

2. The method of item 1, wherein in step (c) untemplated nucleotides are added to the 3′-end of the second oligonucleotide.

3. The method of item 2, wherein second strand DNA synthesis comprises the use of primers comprising a sequence complementary to the added untemplated nucleotides.

4. The method of item 2, wherein a primer comprising RNA nucleotides complementary to the added untemplated nucleotides is added for extension.

5. The method of item 1, wherein second strand DNA synthesis comprises
(a) introducing nicks in the first oligonucleotide;
(b) extending nicked oligonucleotides; and
(c) ligating extended oligonucleotides.

6. The method of item 1 or 5, further comprising subsequent to or concurrently with second strand DNA synthesis a step of introducing untemplated nucleotides at the 5′-end of the synthesized second strand DNA.

7. The method of item 6, wherein untemplated nucleotides are introduced using a transposase enzyme, in particular Tn5 transposase.

8. The method of item 1, wherein the method further comprises a step of linear extension subsequent to DNA ligation, wherein linear extension comprises adding a primer comprising RNA nucleotides and adding a reverse transcriptase enzyme.

9. The method of item 1, wherein the method further comprises a step of linear extension comprising adding a primer comprising random nucleotides.

10. The method of any one of items 1 to 9, wherein the sequence of the first oligonucleotide bound by the first sequence of the second oligonucleotide is located at the 3′-end of the first oligonucleotide.

11. The method of any one of items 1 to 10, wherein the first sequence of the second oligonucleotide is complementary to the 3′ poly-A tail of the first oligonucleotide.

12. The method of any one of items 1 to 11, wherein the first reaction compartment comprises permeabilized intact cells and/or nuclei.

13. The method of any one of items 1 to 12, wherein the first reaction compartment comprises 5000 to 10000 cells.

14. The method of any one of items 1 to 13, wherein the second reaction compartment comprises lysed cells and/or nuclei.

15. The method of any one of items 1 to 14, wherein the second reaction compartment comprises more than one cell and/or nuclei per microbead, preferably 10 cells/nuclei per microbead.

16. The method of any one of items 1 to 15, wherein the second reaction compartment is a microfluidic droplet or a well on a microtiter plate, in particular a sub-nanoliter well plate.

17. The method of item 16, wherein the second reaction compartment is a microfluidic droplet and the third oligonucleotide is released from the microbead upon formation of the droplets.

18. The method of any one of items 1 to 17, wherein the second oligonucleotide further comprises a unique molecular identifier (UMI).

19. The method of any one of items 1 to 18, wherein the cells and/or nuclei are obtained from in vitro cultures or fresh or frozen samples.

20. The method of any one of items 1 to 19, wherein the cells/nuclei are
(a) obtained from existing cell lines, primary cells, blood cells, somatic cells, derived from organoids or xenografts;
(b) CAR-T cells, CAR-NK cells, modified T-cells, B-cells, NK cells, immune cells, or isolated from patients treated with such products; or
(c) pluripotent stem cells (iPS) or embryonic stem cells undergoing natural differentiation or artificially induced reprogramming or transdifferentiation.

21. The method of any one of items 1 to 20, wherein DNA ligation uses a thermostable DNA ligase.

22. Use of a microfluidic system, in particular to generate microfluidic droplets or to deliver material into a microfluidic well-based device, in the method of any one of items 1 to 21.

23. The use of item 22, wherein the microfluidic system is a droplet generator.

24. The use of item 22, wherein the microfluidic system comprises a sub-nanoliter well plate.

25. A kit comprising a second oligonucleotide as defined in item 1, preferably together with instructions regarding the use of the method of any one of items 1 to 21.

26. The kit of item 25 further comprising a transposase enzyme.

27. The kit of item 25 further comprising second strand synthesis reagents and/or a thermostabe ligase.

28. The kit of any one of items 25 to 27 further comprising the fourth oligonucleotide.

The present invention relates to a method for sequencing oligonucleotides comprising RNA, the method comprising the steps of (a) providing permeabilized cells and/or nuclei comprising a first oligonucleotide comprising RNA; (b) combining said cells and/or nuclei of (a) with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide; (c) reversely transcribing the first oligonucleotide in said cells/nuclei to obtain an elongated second oligonucleotide; (d) combining said cells and/or nuclei obtained in step (c) with a microbead-bound third oligonucleotide in a second reaction compartment, wherein the third oligonucleotide comprises (i) a first sequence corresponding to a fourth sequence comprised in the second oligonucleotide used in step (b); or (ii) a first sequence complementary to a first sequence of a fourth oligonucleotide, wherein the fourth oligonucleotide further comprises a second sequence at least partially complementary to the third sequence of the second oligonucleotide; wherein for (i), the method further comprises a step of second strand DNA synthesis subsequent to step (c) and prior to step (d) and wherein for (ii) the method further comprises a step of DNA ligation; and wherein the third oligonucleotide further comprises a second sequence comprising an indexing sequence and a third sequence comprising a primer binding site; (e) amplifying the DNA oligonucleotides obtained in step (d); and (f) sequencing of amplified DNA oligonucleotides. The present method(s) as provided herein may also comprise an additional step of fixation of the permeabilized cells and/or nuclei comprising said first oligonucleotide comprising RNA. Corresponding embodiments are also provided herein below.

The present inventors have surprisingly found that microfluidic scRNA-seq could be used at full capacity when entire transcriptomes are pre-indexed with a first barcode prior to the microfluidic run (FIG. 1b). Even if multiple cells end up in the same droplet and receive the same second microfluidic barcode, their transcriptomes can still be deconvoluted using the first barcode. Importantly, this concept is entirely different from cell hashing with DNA-labelled antibodies (Stoeckius et al. (2018) Genome Biol. 19, 224) or lipids (McGinnis et al. (2019) Nature Methods 16, 619-626). In the case of cell hashing, the cellular transcriptome is never barcoded. Therefore, cell doublets can only be detected but not resolved and have to be discarded for the analysis.

The herein provided method for single-cell RNA sequencing at ultra-high throughput is named scifi-RNA-seq (for: single-cell combinatorial indexing with fluidic indexing RNA sequencing). The method of the invention extends state-of-the-art droplet-based scRNA-seq by single-round combinatorial pre-indexing and thereby increases the throughput by at least 15-fold, at least 20-fold, at least 25-fold or more. This is mainly achieved due to possible loading of multiple cells into one droplet without creating indistinguishably labelled readouts.

In scifi-RNA-seq (FIG. 1b), cells or nuclei are permeabilized and their transcriptomes are pre-indexed by reverse transcription in split pools (i.e., in many physically separated bulk aliquots on microwell plates, for example containing 384 pre-indexing (round1) barcodes). Next, cells or nuclei containing pre-indexed cDNA are pooled, randomly mixed, and encapsulated using a microfluidic droplet generator, such that most droplets are filled and multiple cells or nuclei occupy the same droplet. Inside the droplets, transcripts are labeled with a microfluidic (round2) barcode. Importantly, neither of the two barcodes is exclusive to a cell but shared between all cells in the respective reaction compartment (plate well in round1, droplet in round2). Still, because cells or nuclei are randomly mixed between barcoding rounds, the combination of the two barcodes uniquely identifies single cells.

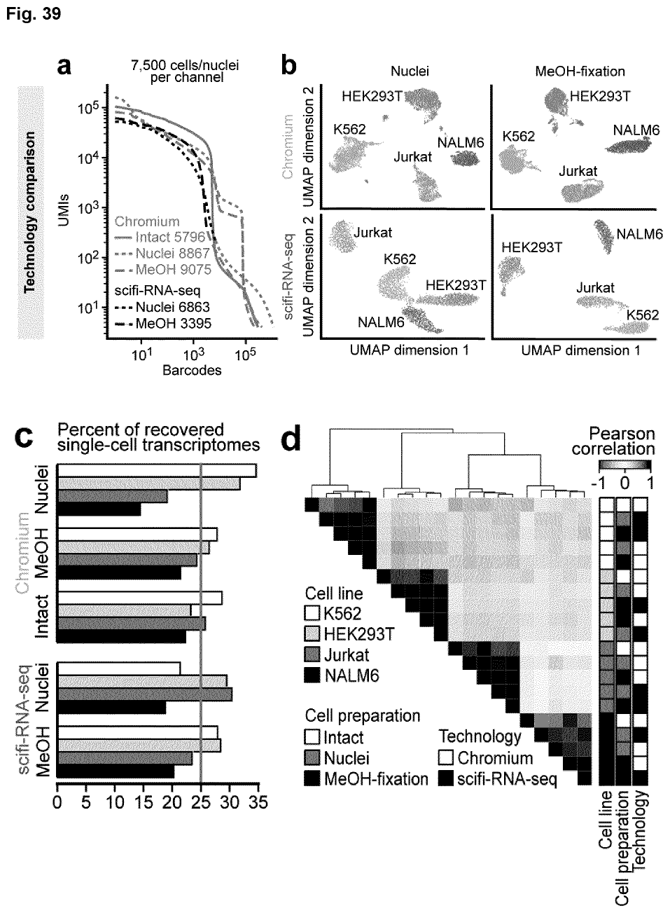

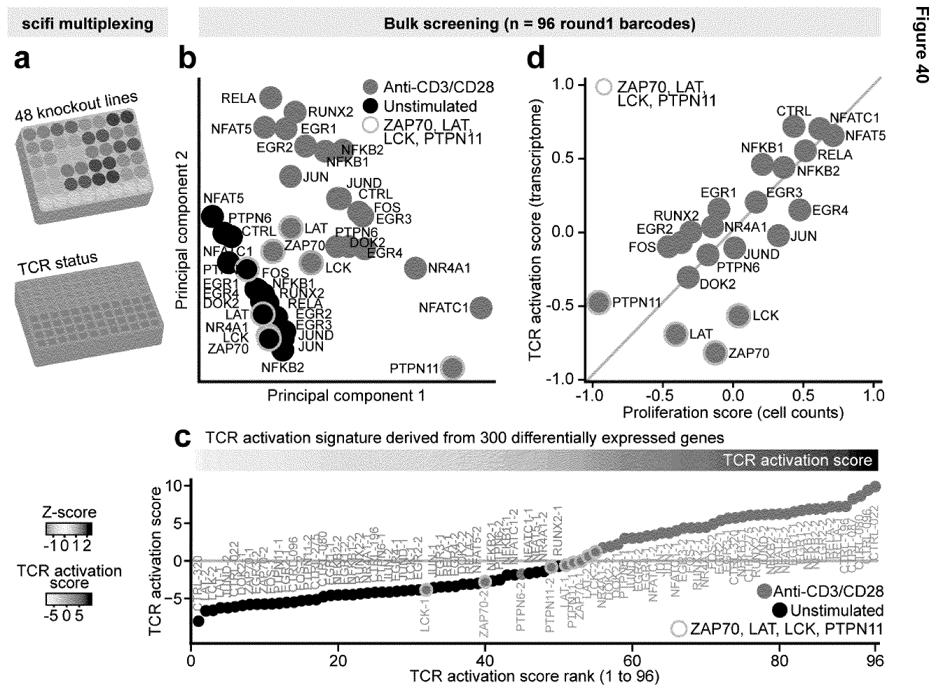

The herein provided means and methods can, inter alia, be used on the Chromium platform commercialized by 10× Genomics (“Chromium™”), which is currently the most popular scRNA-seq platform. However, the method(s) of the invention can be adopted to boost the throughput of any microfluidic or plate-based platforms, in particular nano and/or sub-nanoliter microplate-based platforms, and/or any protocols involving barcoding, like combinatorial indexing protocols. For example, the methods of the invention can be used to improve results obtained using the BectonDickinson Rhapsody system (see e.g. Shum et al. (2019) Adv Exp Med Biol, 1129:63-79/“BD Rhapsody™”). Such an improvement can, inter alia, be seen in a substantially higher cell/nuclei input and/or the potential multiplexing of hundreds or thousands of samples since with the present method no individual channels for assessment are needed. The present invention also provides for cleaner data, like a high single-cell purity. Moreover, the inventors have shown that the method(s) of the invention solve various drawbacks of the standard method(s) used on prior art systems, like the above mentioned Chromium™ platform of 10× Genomics. These surprising ameliorations over the prior art, like Chromium™, comprise for example, reduced “backgrounds” (which are often due to free-floating RNA or cell preparation artefacts) and/or improved (single-)cell purity (as inter alia, illustrated in FIG. 39, for example FIG. 39 a and/or b).

As such, the scifi-RNA-seq method as provided herein and variations thereof, i.e. the methods of the present invention can be used, inter alia, in organ-scale and/or organism-scale single-cell sequencing projects (e.g. Human Cell Atlas) and/or developmental studies at the organ and/or organism level. The methods of the present invention can also be used for the identification of extremely rare and/or transient cell types, developmental stages and/or cellular phenotypes. Such applications may include the identification of extremely rare reprogramming and/or transdifferentiation events that are so far difficult to capture with selectable marker proteins. In a further application of the methods of the present invention, CRISPR single-cell sequencing (e.g. by CROP-seq, Perturb-seq, CRISP-seq, Mosaic-seq) with combined whole transcriptome and/or CRISPR gRNA readout may be envisaged. As a further example, CRISPR single-cell sequencing (e.g. by CROP-seq, Perturb-seq, CRISP-seq, Mosaic-seq) with combined single transcript and CRISPR gRNA readout, or transcript panel and CRISPR gRNA readout may be done using the methods of the present invention. Furthermore, a combination of scifi-RNA-seq and CRISPR single-cell sequencing with CRISPR activation, to profile the response of the whole transcriptome, or a subset of the transcriptome to a perturbation is envisaged. The scifi-RNA-seq method as provided herein and variations thereof, i.e. the methods of the present invention, may also be employed in the drug screenings and/or the testing of compounds, for example the testing of (a) compound(s) for its/their capacity to elucidate a chance in the cellular expression profile and the like. Accordingly, the present invention also provides for screening methods. The means and methods provided herein are also useful in biological/biochemical research approaches, like, inter alia, in the elucidation of ligand-receptor relationships and/or of signal-cascades and their (cellular) consequences.

The methods of the present invention, scifi-RNA-seq, may serve as a readout for CRISPR single-cell sequencing with multiple perturbations per cell, where ultra-high throughput is required to capture all possible combinations.

The methods of the present invention may be combined with single-cell ATAC-seq for integrated transcriptome/epigenome readout. The methods of the present invention may also be combined with lineage tracing methods, for an integrated readout of lineage information and/or transcriptome.

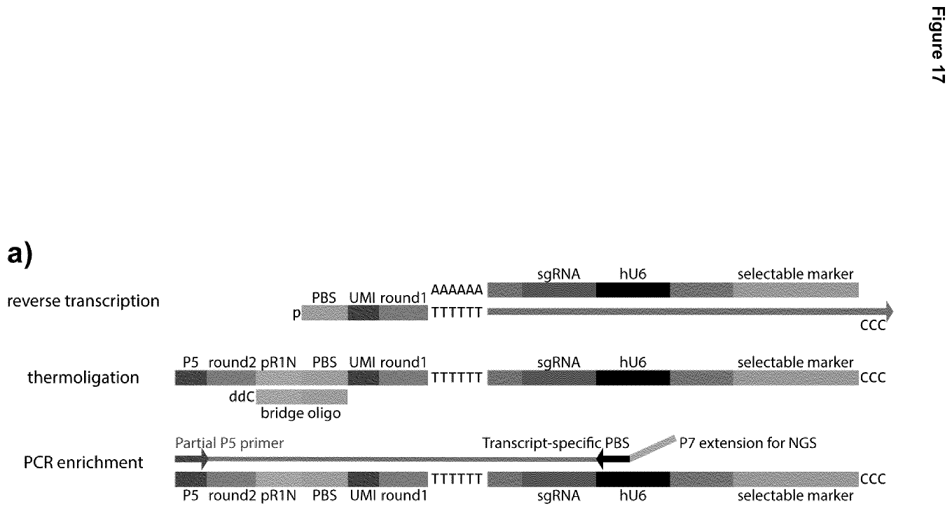

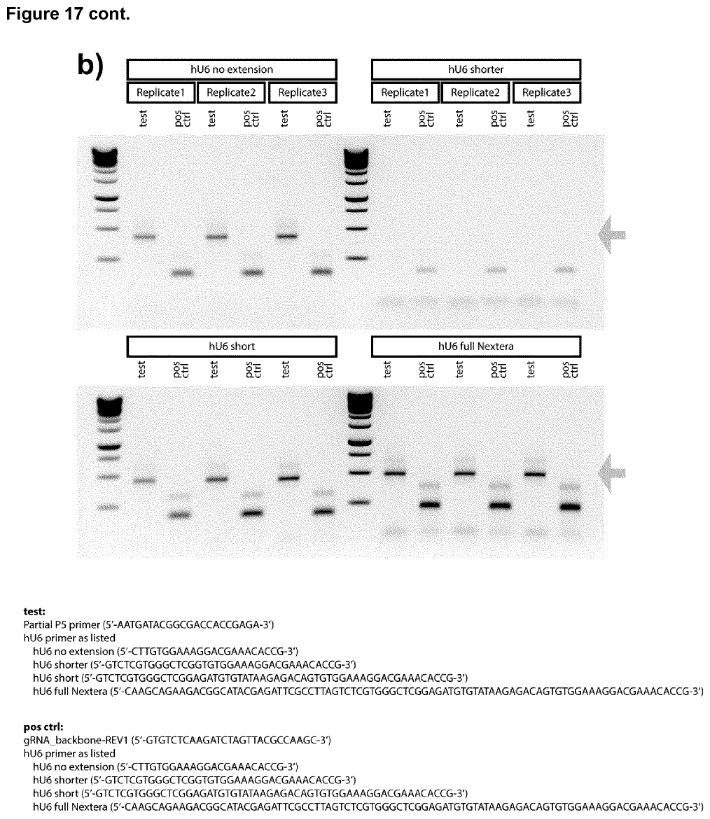

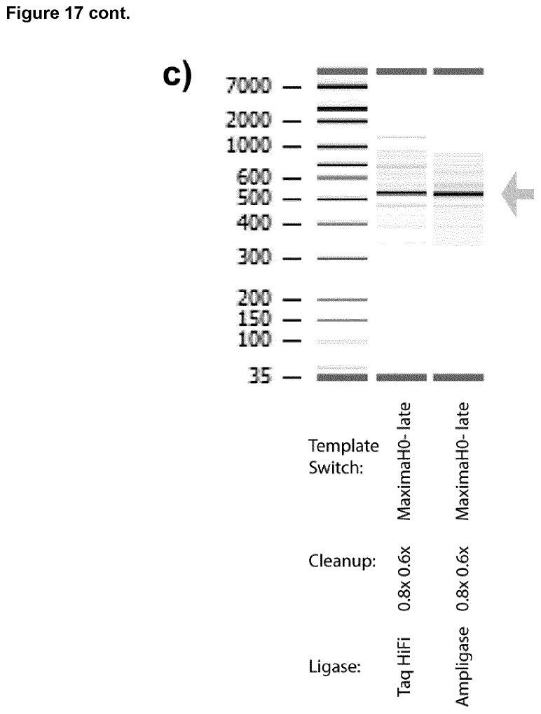

Further provided is the use of scifi-RNA-seq, the methods of the present invention, for immune repertoire sequencing at ultra-high throughput, by specific enrichment of transcripts encoding for the B cell receptor, T cell receptor, or other relevant proteins (FIG. 17).

Also provided is the use of the methods of the present invention, scifi-RNA-seq, for integrated transcriptome and immune repertoire sequencing.

Further provided is the use of the methods of the present invention, scifi-RNA-seq and variations thereof, for the identification of antigen-specific, reactive T-cells, B-cells and/or other immune cells, for example, by means of their activation signature. Also provided is the use for the detection of barcoded antibodies or other biomolecules interacting with extracellular and/or intracellular partners such as targets and/or antigens.

Also provided is the combination of the methods of the present invention with the enrichment of transcripts of interest (single transcripts, panels of transcripts, CRISPR gRNAs, feature barcodes obtained inter alia from barcoded antibodies or other biomolecules), for instance by specific PCR or transcript capture. This includes diagnostic applications.

The means and methods of the present invention are also useful in the assessment of cell-cell interactions and/or in cell-cell interaction profiling. In accordance with this embodiment of the invention the cells are not separated but allowed to physically interact. Cell-cell interactions will allow cells to pass through the same first reaction compartment. Interactions between cells can be stabilized by fixation methods.

Specifically, in a first experiment, the loading capacity of the microfluidic system was tested by substituting the lysis reagents for standard EB buffer. Thus, the number of nuclei contained in the microfluidic droplets could be counted under a light microscope. As shown in FIG. 7, 15,300; 191,250; 382,500; 765,000 and 1,530,000 cell nuclei were loaded per microfluidic channel. Surprisingly, all tested conditions, which constituted massive overloading of the device, resulted in a stable droplet emulsion without clogging of the microfluidic channels, even though up to 1,530,000 nuclei were loaded per channel (100-fold the maximum recommended amount). When loading 1,530,000 nuclei per channel, an average of 9.6 nuclei per droplet was observed. Thus, it was demonstrated that the 10× Genomics Chromium platform can tolerate 100-fold higher loading concentrations than are typically used without clogging of the microfluidic channels. A stable droplet emulsion with the desired random loading distribution was thus achieved.

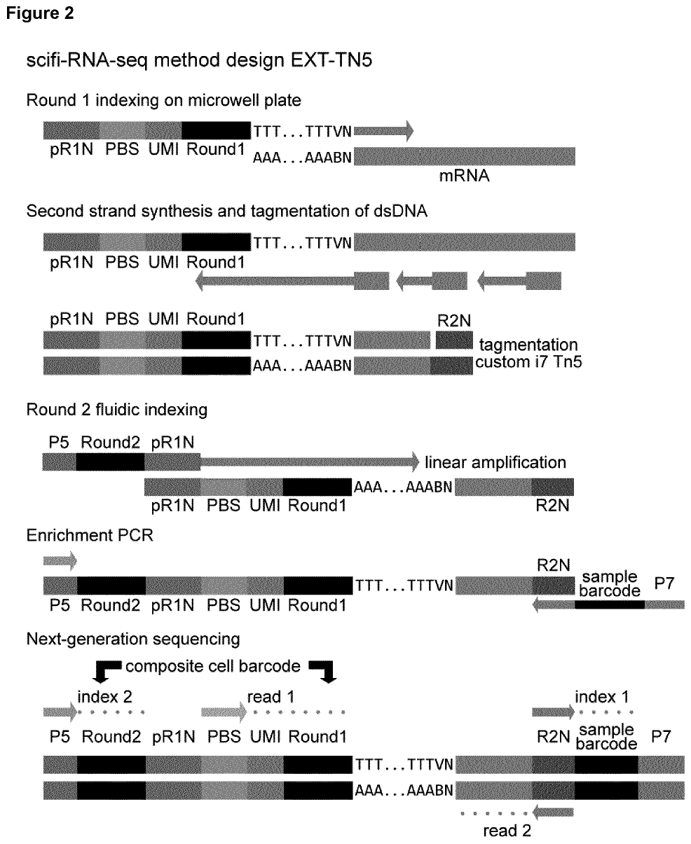

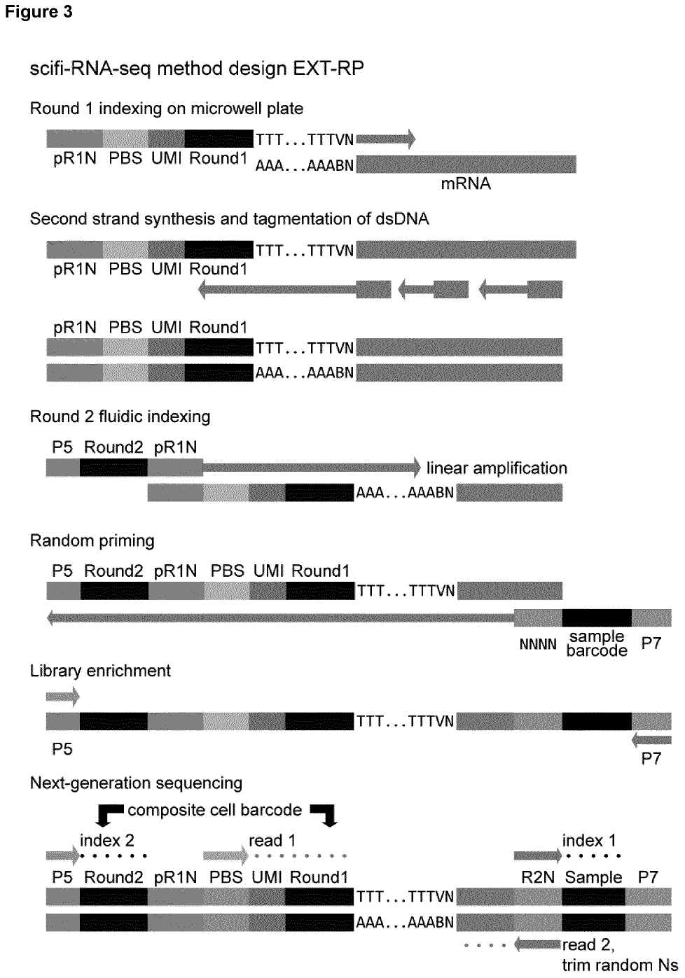

In a second experiment, a first barcode index was introduced using a specialized library preparation method depicted in FIG. 2. Alternative method designs are depicted in FIGS. 3-6. The protocol of the invention works on permeabilized cells and/or nuclei distributed into e.g. a 96-well, 384-well, or 1536-well plate. In this exemplary setup, each well contained a DNA primer containing (1) an oligo-dT stretch for transcript capture, (2) a unique, well-specific round1 index, (3) an optional unique molecular identifier for PCR duplicate removal (4), a primer-binding site for an NGS sequencing primer, and (5) a primer-binding site for a linear barcoding (pR1N) in the microfluidic device. After reverse transcription, RNase H was utilized to introduce nicks into the template mRNA, a DNA polymerase extended the nicks and a DNA ligase sealed them, resulting in double-stranded cDNA.

The next step in this exemplary protocol of the method of the invention was to introduce a second defined end for the ensuing enrichment PCR reaction. This was achieved using a custom Tn5 transposase loaded with an Illumina-compatible i7-only adapter. Alternative means in the methods of the invention to achieve the same outcome are, inter alia, template switching by the reverse transcriptase when provided with an appropriate oligonucleotide; random priming with Klenow Exo- or a similar enzyme; single-stranded ligation with or without RNA base tailing.

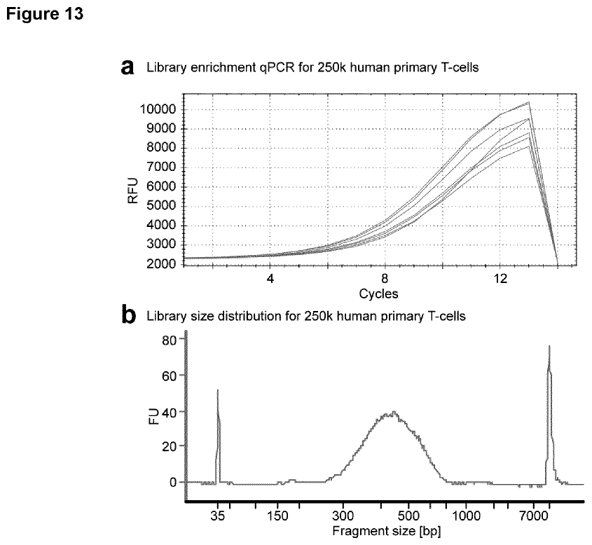

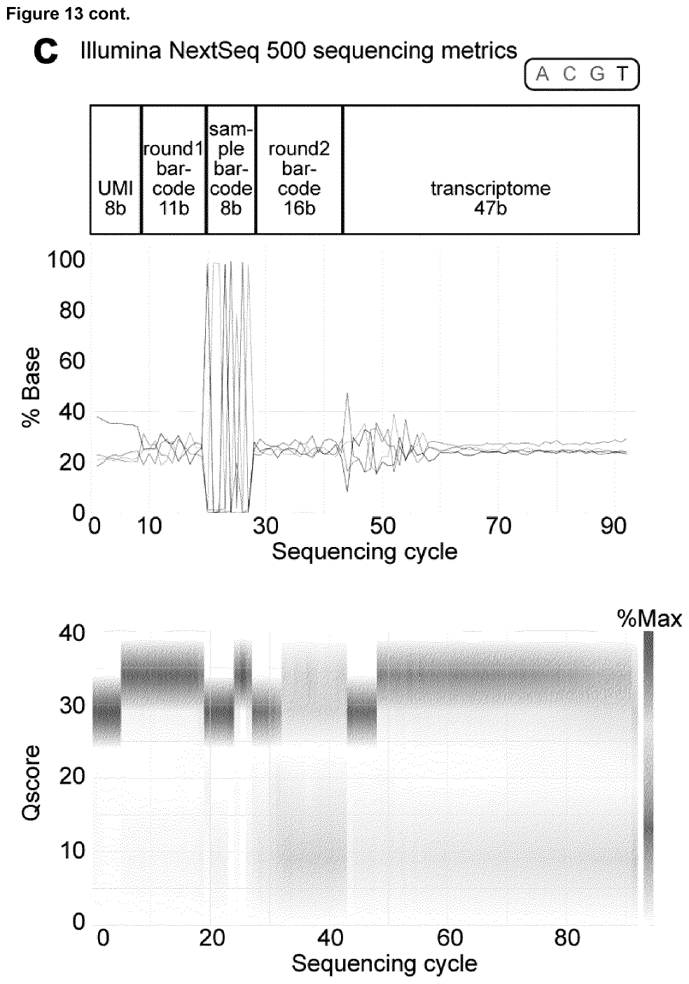

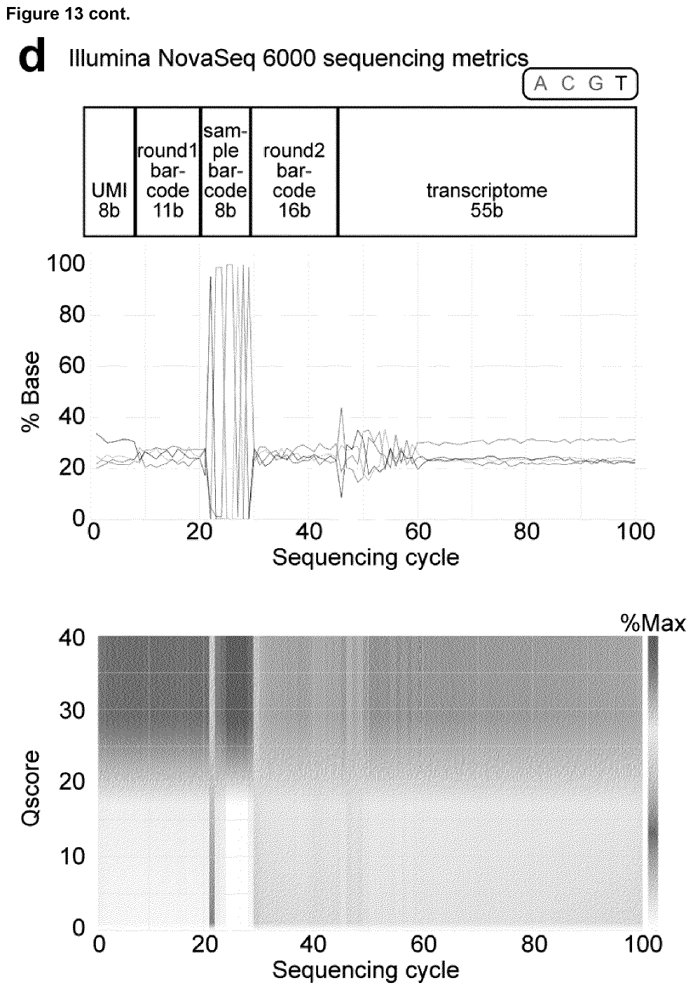

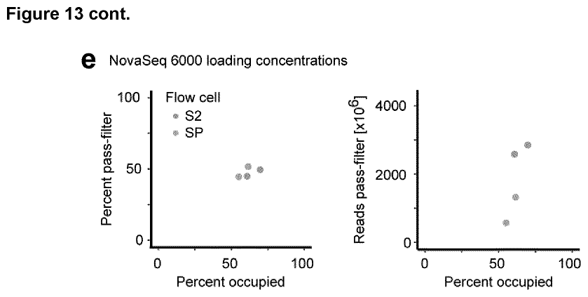

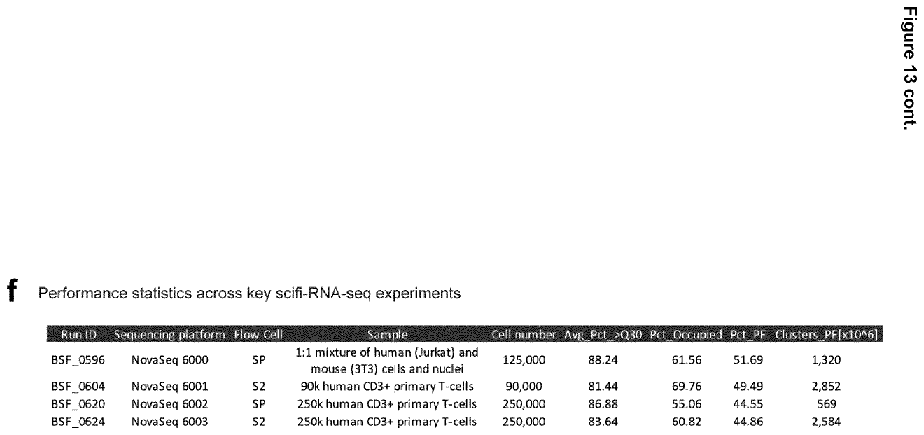

Importantly and advantageously over methods of the prior art, throughout the process, nuclei and/or cells remain intact, and are loaded onto the microfluidic device at an unusually high concentration to promote loading of multiple cells per droplet. In the methods of the invention, one microbead is co-encapsulated with multiple barcoded cells/nuclei. Due to the buffer composition, nuclei are lysed and annealing of the transcriptomes to the microbead-tethered oligos is allowed. The microfluidic droplets were then subjected to multiple rounds of linear extension to introduce the second (microfluidic) barcode into the transcriptomes. After this reaction, the droplet emulsion was broken and the sequencing library was PCR-enriched, which allowed the introduction of an additional, channel-specific barcode. While both the first and second barcodes can be shared by multiple cells, the combination of the two barcodes is unique for an individual cell. During the bioinformatic analysis, cells were identified by their cell barcode comprising both the plate-based first and the microfluidic second barcodes. The combination of both led to the surprising results provided herein. Specifically, the results of a typical library preparation experiment are depicted in FIGS. 13a and 13b. Sequencing metrics for the Illumina NextSeq 500 and NovaSeq 6000 platforms are shown in FIGS. 13c and 13d.

For several reasons, it was believed in the art that combinatorial indexing RNA-seq could not be combined with droplet microfluidics. Most importantly, it was believed that subjecting cells or nuclei to reverse transcription, second strand synthesis, and tagmentation is inevitably damaging. It was thus surprising and unexpected that the methods of the invention lead to a significant improvement over the prior art methods.

In the appended examples, it is shown that the 10× Genomics Chromium assay can be overloaded with 100-fold higher nuclei amounts as maximally recommended. Surprisingly, stable droplet emulsions were achieved without clogging of the microfluidic channels even at the highest loading concentration. Detailed metrics on the nuclei fill rate over a range of high loading concentrations are provided, and it is demonstrated that it can be tightly controlled even at unusually high loading concentrations. For instance, a stable mean fill rate of 9.6 cells per droplet was achieved when loading 1.53 million nuclei per channel (100× the maximum recommended amount). It is also shown that there is no physical limit to filling droplets with nuclei. For instance, loading 1.53 million nuclei per channel resulted in a fill rate of 95.5%.

Moreover, it is shown in the appended examples that nuclei subjected to a combinatorial pre-indexing round are sufficiently stable to withstand the pressure and shear stress inside a microfluidic device. This was unexpected, as they are in some instances of the present invention subjected to three enzymatic reactions: reverse transcription, second strand synthesis, and tagmentation. These steps involve high-temperature incubations and aggressive buffers that were expected to compromise the integrity of nuclei. It was therefore not obvious to combine a pre-indexing step with microfluidics. Surprisingly, the optimized workflow for scifi-RNA-seq as provided herein recovers pre-indexed cells/nuclei at a rate comparable to standard microfluidic scRNA-seq.

The methods of the invention constitute the first use of linear barcoding for single-cell transcriptome sequencing. In some instances, the present invention also provides the first use of a thermostable ligase for next-generation sequencing library preparation. Linear barcoding refers to the introduction of a cell barcode by annealing to a bead-tethered oligonucleotide followed by linear extension with a suitable DNA polymerase. While linear barcoding has been recently described for single-cell ATAC-seq, it has not been suggested for scRNA-seq. There is no other scRNA-seq method using linear barcoding prior to the present invention. Through the invention as described herein, it was demonstrated that linear barcoding is effective for preparing single-cell transcriptome libraries. The resulting data is of high quality and complexity, with minimal technical noise or sequencing artefacts. Similarly, there is no other scRNA-seq method using a thermostable ligase prior to the present invention. For the relevant methods provided herein, it was demonstrated that use of a thermostable ligase is effective for preparing single-cell transcriptome libraries. The resulting data is of high quality and complexity, with minimal technical noise or sequencing artefacts.

By employing droplet microfluidics for the second index, about 750,000 sequences can be used for the second combinatorial barcoding round in the methods of the invention. This results in roughly 288 million barcode possibilities when using a 384-well plate for the first indexing round (384×750,000). Two rounds of state-of-the-art combinatorial indexing in 384-well plates only results in 147,456 combinations. The combination of combinatorial indexing and microfluidic droplet generators also enables scaling of NGS protocols that-due to their design—are not immediately compatible with three rounds of indexing.

In summary, in the methods of the present invention, a pre-indexing step is used to barcode entire single-cell transcriptomes prior to the microfluidic run. The methods of the invention are not subject to the aforementioned limitation because cells can be distinguished even if they enter the same droplet. Thus, microfluidic droplet generators (but also sub-nanoliter well plates) can be loaded with a much higher number of cells than in existing protocols.

As such, the methods of the present invention can be used, inter alia, as a high content readout for saturation mutagenesis, for instance for the experimental annotation of genetic variants in cells. The methods of the present invention can also be used as a high content readout for synthetic biology, e.g. when a large number of synthesized DNA modules are introduced into cells, both natural and artificial.

Accordingly, the present invention, in a first embodiment, relates to a method for sequencing oligonucleotides comprising RNA, the method comprising the steps of (a) providing permeabilized cells and/or nuclei comprising a first oligonucleotide comprising RNA; (b) combining said cells and/or nuclei of (a) with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide; (c) reversely transcribing the first oligonucleotide in said cells/nuclei to obtain an elongated second oligonucleotide; (d) combining said cells and/or nuclei obtained in step (c) with a microbead-bound third oligonucleotide in a second reaction compartment, wherein the third oligonucleotide comprises (i) a first sequence corresponding to a fourth sequence comprised in the second oligonucleotide used in step (b); or (ii) a first sequence complementary to a first sequence of a fourth oligonucleotide, wherein the fourth oligonucleotide further comprises a second sequence at least partially complementary to the third sequence of the second oligonucleotide; wherein for (i), the method further comprises a step of second strand DNA synthesis subsequent to step (c) and prior to step (d) and wherein for (ii) the method further comprises a step of DNA ligation; and wherein the third oligonucleotide further comprises a second sequence comprising an indexing sequence and a third sequence comprising a primer binding site; (e) amplifying the DNA oligonucleotides obtained in step (d); and (f) sequencing of amplified DNA oligonucleotides. As discussed herein the permeabilized cells and/or nuclei comprising a first oligonucleotide comprising RNA may also be fixed, for axmaple via chemical cross linking of the RNA to be analyzed on or to cellular structures or on or to structures of the nuclei. Details of this embodiment of an additional fixing step are also provided herein below. The fixation step may in particular of interest when fresh samples, like, non-preserved cells/nuclei (e.g. material that is previously not formalin-fixed) is to be analyzed in accordance with means and methods of the present invention.

Thus, in general, the invention relates to a method for sequencing oligonucleotides comprising RNA. The term “sequence” refers to sequence information about an oligonucleotide or any portion of the oligonucleotide that is two or more units (nucleotides) long. The term can also be used as a reference to the oligonucleotide itself or a relevant portion thereof.

Oligonucleotide sequence information relates to the succession of nucleotide bases in the oligonucleotide, in particular RNA, in particular RNA of the first oligonucleotide as in the methods of the present invention. For example, if the oligonucleotide contains bases Adenine, Guanine, Cytosine, and/or Uracil, or chemical analogs thereof, the oligonucleotide sequence can be represented by a corresponding succession of letters A, G, C, or U, respectively. Such oligonucleotides may be sequenced using the methods of the present invention.

Accordingly, in a first step, the methods of the invention comprise a step of providing permeabilized cells and/or nuclei comprising a first oligonucleotide comprising RNA. The first oligonucleotide comprises RNA. However, the methods of the present invention are not limited by the type of RNA of the first oligonucleotide or as comprised in the cells/nuclei used in the methods of the invention. Thus, the RNA may of any type known to the person skilled in the art. The RNA may preferably be messenger RNA. It may preferably represent parts or the entirety of the transcriptome as comprised in the cells/nuclei used in the methods of the present invention, preferably the transcriptome in its entirety. As such, the RNA comprised in the first oligonucleotide is preferably in the form of messenger RNA (mRNA). As the skilled person will appreciate, mRNA generally comprises a polyadenylated tail at its 3′ end. Accordingly, it is preferred that the first sequence of the second oligonucleotide is at least partially complementary to the 3′ end of the first oligonucleotide, i.e. the poly-A-tail. However, the methods of the present invention are not limited to binding to the 3′ end. Rather, the first sequence of the second oligonucleotide can be at least partially complementary to a sequence of the first oligonucleotide, wherein said sequence is located in 5′ direction from the 3′ end of the first oligonucleotide. This can, inter alia, be used in cases where the target sequence is known or at least partially known.

The cells/nuclei may be present in various states and may be obtained from samples of various states or origins.

For example, in one embodiment, the cells and/or nuclei are obtained from in vitro cultures or fresh or frozen samples. Cells/nuclei might be obtained from preserved tissue samples, such as formalin-fixed paraffin-embedded (FFPE) material.

Within the present invention, the cells/nuclei may be of any origin as long as the cells/nuclei comprise oligonucleotides comprising RNA. For example, the cells may be cell lines, primary cells, blood cells, somatic cells, derived from organoids or xenografts. Furthermore, cells might be obtained from cell preparations used in immune oncology such as, for example, CAR-T cells, CAR-NK cells, modified T cells, B cells, NK cells or other immune cells, or isolated from patients treated with such products. Moreover, cells might be induced pluripotent stem cells (iPS) or embryonic stem cells undergoing natural differentiation or artificially induced reprogramming or transdifferentiation. Accordingly, the nuclei may be derived from any of the above cells, including e.g. blood cells, somatic cells, induced pluripotent stem cells (iPS) or embryonic stem cells. As such, the methods of the present invention can, inter alia, be used in immune oncology (CAR-T cells, CAR-NK cells, bispecific engagers, BiTEs, immune checkpoint blockade, cancer vaccines delivered as mRNA), molecularly targeted cancer therapy, the dissection of drug resistance and toxicity mechanisms and/or target discovery and/or validation.

In further embodiments, the cells and/or nuclei may be obtained from biological material used in forensics, reproductive medicine, regenerative medicine or immune oncology. Accordingly, the cells and/or nuclei may be cells/nuclei derived from a tumor, blood, bone marrow aspirates, lymph nodes and/or cells/nuclei obtained from a microdissected tissue, a blastomere or blastocyst of an embryo, a sperm cell, cells/nuclei obtained from amniotic fluid, or cells/nuclei obtained from buccal swabs. It is preferred that the tumor cells/nuclei are disseminated tumor cells/nuclei, circulating tumor cells/nuclei or cells/nuclei from tumor biopsies. It is furthermore preferred that the blood cells/nuclei are peripheral blood cells/nuclei or cells/nuclei obtained from umbilical cord blood. It is particularly preferred that the RNA oligonucleotides comprised in the cells/nuclei represent the transcriptome of the cells/nuclei.

Within the methods of the present invention, the cells/nuclei are provided in a permeabilized state. The skilled person is well-aware of methods suitable to provide cells/nuclei in said state. For example, methanol permeabilization may be used for whole cells, whereas incomplete lysis with detergents such as Igepal CA-630, Digitonin or Tween-20 may be used. As such, the first reaction compartment may comprise permeabilized intact cells and/or nuclei.

The number of cells in the first reaction compartment is not particularly limited. However, the total number of cells will depend on the lengths chosen for first and second indexing sequences and the number of unique first and second indices in order to ensure proper sample attribution. Typically, in the methods of the present invention, the first reaction compartment comprises 5000 to 10000 cells.

In a second step of the methods of the invention, the cells and/or nuclei comprising the first oligonucleotide comprising RNA are combined with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide.

In a preferred embodiment of the invention, the cells and/or nuclei comprising the first oligonucleotide comprising RNA are combined with a second oligonucleotide comprising DNA in a first reaction compartment, wherein the second oligonucleotide comprises at least a first sequence at least partially complementary to the 3′-end of the first oligonucleotide, a second sequence comprising an indexing sequence, and a third sequence comprising a primer binding site, under conditions to allow annealing of the first sequence of the second oligonucleotide to the 3′-end of the first oligonucleotide.

As detailed further above, the methods of the invention allow a surprisingly high throughput of cells/nuclei to be analyzed/sequenced. This is at least partially due to the introduction of at least two indexing sequences into the oligonucleotide comprising RNA that is to be analyzed/sequenced. The first of said at least two indexing sequences is introduced by combining the cells/nuclei comprising the first oligonucleotide with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide. In a particular embodiment, the first of at least two indexing sequences is introduced by combining the cells/nuclei comprising the first oligonucleotide comprising RNA with a second oligonucleotide comprising DNA in a first reaction compartment, wherein the second oligonucleotide comprises at least a first sequence at least partially complementary to the 3′-end of the first oligonucleotide, a second sequence comprising an indexing sequence, and a third sequence comprising a primer binding site, under conditions to allow annealing of the first sequence of the second oligonucleotide to the 3′-end of the first oligonucleotide.

Accordingly, a second oligonucleotide is employed in the methods of the present invention. The second oligonucleotide comprises DNA and at least three functional sequences/parts. A first sequence of the second oligonucleotide is at least partially complementary to a sequence of the first oligonucleotide, preferably to the 3′end of the first oligonucleotide. As described above, it is preferred within the present invention that the first oligonucleotide comprising RNA comprises a polyadenylated 3′ end, for example as generally comprised in mRNA. Thus, it is preferred that the first sequence of the second oligonucleotide employed in the methods of the present invention comprises a sequence at least partially complementary to the 3′-end of the first oligonucleotide, in particular a sequence predominantly comprising thymine residues or consisting of thymine residues. As such, the first sequence of the second oligonucleotide may partially or completely anneal to the 3′ end of the first oligonucleotide. Provided is thus a method, wherein the first sequence of the second oligonucleotide is complementary to the 3′ poly-A tail of the first oligonucleotide. However, as also provided herein, the methods of the invention are not limited to the first sequence of the second oligonucleotide being at least partially complementary to the poly-A-tail of the first oligonucleotide. The first sequence of the second oligonucleotide can be at least partially complementary to a sequence lying 5′ from the 3′ end of the first oligonucleotide.

The second sequence/part of the second oligonucleotide comprises or consists of an indexing sequence. The term “indexing sequence” is known to the person skilled in the art, although it is surprising that an indexing sequence is used as part of the second oligonucleotide employed in the methods of the invention.

The term “indexing sequence” in accordance with the invention is to be understood as a sequence of nucleotides that is known or may not be known, wherein each position has an independent and equal probability of being any nucleotide. In a preferred embodiment of the methods of the present invention, the first indexing sequence is known and the second indexing sequence may be known or unknown. The nucleotides of the indexing sequence can be any of the nucleotides, for example G, A, C, T, U, or chemical analogs thereof, in any order, wherein: G is understood to represent guanylic nucleotides, A adenylic nucleotides, T thymidylic nucleotides, C cytidylic nucleotides and U uracylic nucleotides. The skilled person will appreciate that known oligonucleotide synthesis methods may inherently lead to unequal representation of nucleotides G, A, C, T or U. For example, synthesis may lead to an overrepresentation of nucleotides, such as G in randomized DNA sequences. This may lead to a reduced number of unique sequences as expected based on an equal representation of nucleotides. However, the skilled person is well aware that the overall number of unique sequences comprised in the second oligonucleotide used in the methods of the invention will generally be sufficient to clearly identify each target RNA comprising oligonucleotide. This is because the skilled person will also be aware of the fact that the length of the indexing sequence may be varied depending on the number of expected first oligonucleotides. The expected number of first oligonucleotides may be derived from the number of genes expected to be expressed and/or the number of cells/nuclei expected to be analyzed/sequenced. Accordingly, the potential unequal representation of nucleotides in the indexing sequence of the second oligonucleotide used in the methods of the invention, which is due to unequal coupling efficiencies of nucleotides in known standard oligonucleotide synthesis methods, can easily be taken into account by the skilled person based on the general knowledge in the art. In particular, the skilled person is well aware that the length of the indexing sequence may be increased in order to obtain an increased number of unique sequences.

The third sequence comprised in the second oligonucleotide used in the methods of the present invention comprises a primer binding site. The skilled person is well aware of suitable sequences. As such, any sequence can be employed as long as a primer employed in the methods of the present invention is allowed to bind to the third sequence of the second oligonucleotide used in the methods of the present invention.

Within the methods of the present invention, the first sequence of the second oligonucleotide is allowed to anneal to a sequence comprised in the first oligonucleotide, preferably to the 3′ end of the first oligonucleotide. The skilled person is well aware of conditions allowing the annealing of these sequences to each other. Within the present invention, the constitution of the first sequence of the second oligonucleotide favours the annealing. Namely, the first sequence of the second oligonucleotide predominantly comprises nucleotides complementary to nucleotides comprised in the target sequence of the first oligonucleotide, preferably constituting the 3′-end of the first oligonucleotide. In a preferred embodiment, the 3′ end of the first oligonucleotide comprises adenine nucleotides and as such will anneal to thymine nucleotides comprised in the first sequence of the second oligonucleotide.

In certain embodiments of the invention, the second oligonucleotide further comprises a unique molecular identifier (UMI).

Subsequent to annealing of the first sequence of the second oligonucleotide to the first oligonucleotide, preferably to the 3′ end of the first oligonucleotide, the methods of the present invention comprise a step of reversely transcribing the first oligonucleotide in said cells/nuclei to obtain an elongated second oligonucleotide. The skilled person is well-aware of means and methods that can be employed to reversely transcribe the first oligonucleotide within the methods of the present invention. More specifically, the reaction will generally involve the use of a reverse transcriptase enzyme. In certain embodiments of this invention a reverse transcriptase with the ability to add untemplated nucleotides might be preferred.

Reverse transcriptases are enzymes composed of distinct domains that exhibit different biochemical activities. RNA-dependent DNA polymerase activity and RNase H activity are the predominant functions of reverse transcriptases, although depending on the source organisms there are variations in functions, including, for example, DNA-dependent DNA polymerase activity. The reverse transcription process typically involves a number of steps:

In the presence of an annealed primer, reverse transcriptase binds to an RNA template and initiates the reaction. RNA-dependent DNA polymerase activity synthesizes the complementary DNA (cDNA) strand, incorporating dNTPs. Optional RNase H activity degrades the RNA template of the DNA:RNA complex. DNA-dependent DNA polymerase activity (if present) recognizes the single-stranded cDNA as a template, uses an RNA fragment as a primer, and synthesizes the second-strand cDNA to form double-stranded cDNA. In the methods of the present invention, various types of reverse transcriptase enzymes can be used, in particular enzymes having RNA-dependent DNA polymerase activity only or enzymes having RNA-dependent DNA polymerase activity combined with RNase H activity. Enzymes having all of the above three activities may also be used.

For example, the method may be carried out by incubating the first reaction compartment, for example a multi-well plate, for a given time at an elevated temperature, for example for 5 or more minutes at about 55° C., such that RNA secondary structures are resolved. Subsequent to resolving secondary structures, the first reaction compartment may be placed on ice to prevent their re-formation. Then, a reaction mix comprising buffer, dNTPs and a reverse transcription enzyme may be added to initiate the reverse transcription reaction. Additives such as RNase inhibitors or DTT might be added to the reaction. Preferably, the reaction is carried out at increasing temperatures starting with about 4° C. and gradually increasing the temperature to about 55° C.

Certain reverse transcriptases may also display terminal nucleotidyl transferase (TdT) activity, which results in non-template-directed addition of nucleotides to the 3′ end of the synthesized DNA. TdT activity occurs only when the reverse transcriptase reaches the 5′ end of the RNA template, adds extra nucleotides to the cDNA end, and exhibits specificity towards double-stranded nucleic acid substrates (e.g., DNA:RNA in the first-strand cDNA synthesis and DNA:DNA in the second-strand cDNA synthesis). An exemplary reverse transcriptase enzyme having such activity is Maxima H Minus RT. While this activity is oftentimes undesirable because the added nucleotides do not correspond to the template, the methods of the invention may comprise the use of such enzymes. As such, in a particular embodiment, the methods of the invention comprise a step (c), wherein untemplated nucleotides are added to the 3′-end of the second oligonucleotide. In a more particular embodiment of the invention, second strand DNA synthesis may then comprises the use of primers comprising a sequence complementary to the added untemplated nucleotides.

Accordingly, subsequently to reverse transcription, the methods of the invention may comprise a step of second strand DNA synthesis to obtain double-stranded cDNA.

Subsequent to reverse transcription and/or second strand DNA synthesis, the methods of the invention comprise the transfer of the permeabilized cells/nuclei to a second reaction compartment. At this stage, the cells/nuclei are permeabilized but preferably still intact, that is non-lysed. As such, the methods of the present invention allow using permeabilized intact cells/nuclei during the first indexing reaction, whereas methods of the prior art comprise a lysis step prior to the first indexing reaction.

The second reaction compartment may be a microfluidic droplet or a microtiter plate. The microtiter plate may be a miniaturized microtiter plate. In another embodiment of the invention, both the first and second reaction compartment may be generated by a microfluidic droplet generator or may be a miniaturized plate. Within the present invention, both reaction compartments may also be standard microwell plates. Exemplary plates include Seq-Well (Gierahn et al. (2017) Nature Methods 14, 395-8) or Microwell-seq (Han et al. (2018) Cell 172(5), 1091-1107).

In the second reaction compartment, the cells and/or nuclei obtained in step (c) are combined with a microbead-bound third oligonucleotide, wherein the third oligonucleotide comprises
(i) a first sequence corresponding to a fourth sequence comprised in the second oligonucleotide used in step (b); or
(ii) a first sequence complementary to a first sequence of a fourth oligonucleotide, wherein the fourth oligonucleotide further comprises a second sequence at least partially complementary to the third sequence of the second oligonucleotide;
wherein for (i), the method further comprises a step of second strand DNA synthesis subsequent to step (c) and prior to step (d) and wherein for (ii) the method further comprises a step of DNA ligation;
and wherein the third oligonucleotide further comprises a second sequence comprising an indexing sequence and a third sequence comprising a primer binding site.

The cells/nuclei may be lysed subsequent to transfer to the second reaction compartment. As such, the second reaction compartment may comprise lysed cells/nuclei.

The third oligonucleotide used in the methods of the present invention comprises at least three functional parts/sequences and is initially bound to a microbead. In the second reaction compartment, the microbead may be dissolved and the third oligonucleotide released. A first sequence comprised in the third oligonucleotide is used to either directly or indirectly direct the cDNA comprised in the cells/nuclei obtained in the previous method steps to the microbead-bound third oligonucleotide.

Whether the first sequence of the third oligonucleotide binds the cDNA directly or indirectly depends on the presence of a second strand DNA synthesis step prior to combining the cDNA with the microbead-bound third oligonucleotide. In one embodiment, the first sequence of the third oligonucleotide may correspond to a fourth sequence part of the second oligonucleotide. As the skilled person will appreciate, a sequence corresponding to a part of the second oligonucleotide will be complementary to the synthesized second strand DNA. As such, this embodiment of the invention comprises a step of second strand DNA synthesis subsequent to step (c) and prior to step (d).

In a preferred embodiment of the invention, second strand DNA synthesis comprises introducing nicks in the first oligonucleotide; extending nicked oligonucleotides; and ligating extended oligonucleotides. The nicks may be introduced by addition of a further enzyme, for example RNase H. As detailed above, the reverse transcriptase enzyme may have RNase H activity and may thus also be used to introduce nicks in the first oligonucleotide. The nicked oligonucleotides are then extended by the reverse transcriptase enzyme and/or a further enzyme such as a DNA polymerase and are subsequently ligated to form cDNA oligonucleotides for further processing.

The methods of the present invention may further comprise subsequent to or concurrently with second strand DNA synthesis a step of introducing untemplated nucleotides at the 5′-end of the synthesized second strand DNA. Preferably, untemplated nucleotides are introduced using a transposase enzyme, in particular Tn5 transposase.

Transposase is an enzyme that binds to the end of a transposon and catalyzes the movement of the transposon to another part of the genome by a cut and paste mechanism or a replicative transposition mechanism. Transposases are classified under EC number EC 2.7.7. Genes encoding transposases are widespread in the genomes of most organisms and are the most abundant genes known. A preferred transposase within the context of the present invention is Transposase (Tnp) Tn5, in particular a customized transposase. Tn5 is a member of the RNase superfamily of proteins which includes retroviral integrases. Tn5 can be found in Shewanella and Escherichia bacteria. The transposon codes for antibiotic resistance to kanamycin and other aminoglycoside antibiotics. Tn5 and other transposases are notably inactive. Because DNA transposition events are inherently mutagenic, the low activity of transposases is necessary to reduce the risk of causing a fatal mutation in the host, and thus eliminating the transposable element. One of the reasons Tn5 is so unreactive is because the N- and C-termini are located in relatively close proximity to one another and tend to inhibit each other. This was elucidated by the characterization of several mutations which resulted in hyperactive forms of transposases. One such mutation, L372P, is a mutation of amino acid 372 in the Tn5 transposase. This amino acid is generally a leucine residue in the middle of an alpha helix. When this leucine is replaced with a proline residue the alpha helix is broken, introducing a conformational change to the C-Terminal domain, separating it from the N-Terminal domain enough to promote higher activity of the protein. Accordingly, it is preferred that such a modified transposase be used, which has a higher activity than the naturally occurring Tn5 transposase. In addition, it is particularly preferred that the transposase employed in the methods of the invention is loaded with oligonucleotides, which are inserted into the target double-stranded oligonucleotide, preferably loaded with untemplated nucleotides.

Accordingly, it is preferred to use a hyperactive Tn5 transposase and a Tn5-type transposase recognition site (Goryshin and Reznikoff, J. Biol. Chem., 273:7367 (1998)), or MuA transposase and a Mu transposase recognition site comprising RI and R2 end sequences (Mizuuchi, K., Cell, 35: 785, 1983; Savilahti, H, et al, EMBO J., 14: 4893, 1995). More examples of transposition systems that can be used in the methods of the present invention include Staphylococcus aureus Tn552 (Colegio et al, J. Bacteriol, 183: 2384-8, 2001; Kirby C et al, Mol. Microbiol, 43: 173-86, 2002), Tyl (Devine & Boeke, Nucleic Acids Res., 22: 3765-72, 1994 and International Publication WO 95/23875), Transposon Tn7 (Craig, N L, Science. 271: 1512, 1996; Craig, N L, Review in: Curr Top Microbiol Immunol, 204:27-48, 1996), Tn/O and IS 10 (Kleckner N, et al, Curr Top Microbiol Immunol, 204:49-82, 1996), Mariner transposase (Lampe D J, et al, EMBO J., 15: 5470-9, 1996), Tel (Plasterk R H, Curr. Topics Microbiol. Immunol, 204: 125-43, 1996), P Element (Gloor, G B, Methods Mol. Biol, 260: 97-1 14, 2004), Tn3 (Ichikawa & Ohtsubo, J Biol. Chem. 265: 18829-32, 1990), bacterial insertion sequences (Ohtsubo & Sekine, Curr. Top. Microbiol. Immunol. 204: 1-26, 1996), retroviruses (Brown, et al, Proc Natl Acad Sci USA, 86:2525-9, 1989), and retrotransposon of yeast (Boeke & Corces, Annu Rev Microbiol. 43:403-34, 1989). More examples include IS5, TnIO, Tn903, IS91 1, and engineered versions of transposase family enzymes (Zhang et al, (2009) PLOS Genet. 5:el000689. Epub 2009 Oct. 16; Wilson C. et al (2007) J. Microbiol. Methods 71:332-5) and those described in U.S. Pat. Nos. 5,925,545; 5,965,443; 6,437,109; 6,159,736; 6,406,896; 7,083,980; 7,316,903; 7,608,434; 6,294,385; 7,067,644, 7,527,966; and International Patent Publication No. WO2012103545, all of which are specifically incorporated herein by reference in their entirety.

While any buffer suitable for the used transposase may be used in the methods of the present invention, it is preferred to use a buffer particularly suitable for efficient enzymatic reaction of the used transposase. In this regard, a buffer comprising dimethylformamide is particularly preferred for use in the methods of the present invention, in particular during the transposase reaction. In addition, buffers comprising alternative buffering systems including TAPS, Tris-acetate or similar systems can be used. Moreover, crowding reagents as polyethylenglycol (PEG) are particularly useful to increase tagmentation efficiency of very low amounts of DNA. Particularly useful conditions for the tagmentation reaction are described by Picelli et al. (2014) Genome Res. 24:2033-2040.

The transposase enzyme catalyzes the insertion of a nucleic acid, in particular a DNA in a target nucleic acid, in particular target DNA. The transposase used in the methods of the present invention is loaded with oligonucleotides, which are inserted into the target nucleic acid, in particular the target DNA. The complex of transposase and oligonucleotide is also referred to as transposome. Preferably, the transposome is a heterodimer comprising two different oligonucleotides for integration. In this regard, the oligonucleotides that are loaded onto the transposase comprise multiple sequences. In particular, the oligonucleotides comprise, at least, a first sequence and a second sequence. The first sequence is necessary for loading the oligonucleotide onto the transposase. Exemplary sequences for loading the oligonucleotide onto the transposase are given in US 2010/0120098. The second sequence comprises a linker sequence necessary for primer binding during amplification, in particular during PCR amplification, optionally further comprising untemplated nucleotides. Accordingly, the oligonucleotide comprising the first and second sequence is inserted in the target nucleic acid, in particular the target DNA, by the transposase enzyme. The oligonucleotide may further comprise sequences comprising barcode sequences. Barcode sequences may be random sequences or defined sequences. In this regard, the term “random sequence” in accordance with the invention is to be understood as a sequence of nucleotides, wherein each position has an independent and equal probability of being any nucleotide. The random nucleotides can be any of the nucleotides, for example G, A, C, T, U, or chemical analogs thereof, in any order, wherein: G is understood to represent guanylic nucleotides, A adenylic nucleotides, T thymidylic nucleotides, C cytidylic nucleotides and U uracylic nucleotides. The skilled person will appreciate that known oligonucleotide synthesis methods may inherently lead to unequal representation of nucleotides G, A, C, T or U. For example, synthesis may lead to an overrepresentation of nucleotides, such as G in randomized DNA sequences. This may lead to a reduced number of unique random sequences as expected based on an equal representation of nucleotides. The oligonucleotide for insertion into the target nucleic acid, in particular DNA, may further comprise sequencing adaptors.

The person skilled in the art is well-aware that the time required for the used transposase to efficiently integrate a nucleic acid, in particular a DNA, in a target nucleic acid, in particular target DNA, can vary depending on various parameters, like buffer components, temperature and the like. Accordingly, the person skilled in the art is well-aware that various incubation times may be tested/applied before an optimal incubation time is found. Other factors may be the ratio of transposomes to tagmented DNA. Optimal in this regard refers to the optimal time taking into account integration efficiency and/or required time for performing the methods of the invention.

The first sequence of the third oligonucleotide may alternatively be complementary to a first sequence of a fourth oligonucleotide present in the second reaction compartment. Accordingly, the third oligonucleotide may comprise a first sequence complementary to a first sequence of a fourth oligonucleotide, wherein the fourth oligonucleotide further s a second sequence at least partially complementary to the third sequence of the second oligonucleotide. The presence of the fourth oligonucleotide directs the second oligonucleotide to the third oligonucleotide. In this embodiment, the second oligonucleotide is then ligated to the third oligonucleotide. As the skilled will appreciate, in this embodiment, the second oligonucleotide comprises a 5′-phosphorylation for ligation. In this embodiment, the fourth oligonucleotide is preferably blocked on its 3′-end to prevent extension by DNA polymerases. Thus, in this embodiment, the method further comprises a step of DNA ligation to obtain an oligonucleotide comprising the second and third oligonucleotide. In a preferred embodiment of this invention, the ligase is thermostable. Exemplary thermostable ligases include, but are not limited to, Ampligase (Lucigen) or Taq HiFi DNA Ligase (New England Biolabs). This allows the use of heat denaturation and cooling, i.e. temperature cycles, to anneal the second, third and fourth oligonucleotides without compromising the activity of the ligase. Specifically, emulsion droplets containing said oligonucleotides and the ligase enzyme can be subjected to multiple rounds of thermal cycling between heat denaturation and annealing, which allows efficient annealing and ligation.

In the methods of the present invention, the third oligonucleotide further comprises a second sequence comprising an indexing sequence and a third sequence comprising a primer binding site. As such, a second indexing sequence is introduced in the methods of the present invention. The combined use of the first and second indexing sequences enables the surprisingly high throughput of cells/nuclei achieved in the methods of the present invention. This is because due to the presence of two independent indexing sequences, the second reaction compartment in the methods of the present invention may comprise more than one cell/nuclei per microbead, preferably 10 cells/nuclei per microbead. Methods of the prior art allow much lower throughput, because the number of cells/nuclei is limited in theory to 1 cell/nuclei per microbead in order to ensure that RNA of a cell/nuclei receives a unique indexing sequence. In practice, methods of the prior art are even further limited due to practical reasons to 0.1-0.2 cells/nuclei per microbead.

The methods of the present invention further comprise a step of amplifying the DNA oligonucleotides obtained by combining the second and third oligonucleotides, optionally together with the fourth oligonucleotide. This step comprises linear extension for incorporation of the second indexing sequence comprised in the third oligonucleotide and amplification for sequencing.

The methods of the invention then comprise a step of sequencing of amplified DNA oligonucleotides.

The skilled person is well-aware of methods suitable to sequence DNA oligonucleotides. Exemplary, non-limiting methods to be used in order to determine the sequence of an oligonucleotide are e.g. methods for sequencing of nucleic acids (e.g. Sanger di-deoxy sequencing), massive parallel sequencing methods such as pyrosequencing, reverse dye terminator, proton detection, phospholinked fluorescent nucleotides or nanopore sequencing.

In particular, the resulting amplified oligonucleotides may be subjected to either conventional Sanger-based dideoxy nucleotide sequencing methods or employing novel massive parallel sequencing methods (“next generation sequencing”) such as those marketed by Roche (454 technology), Illumina (e.g. Solexa technology, sequencing-by-synthesis technology), ABI (Solid technology), Oxford Nanopore (e.g. nanopore sequencing) or Pacific Biosciences (SMRT technology). It is preferred to use the Illumina NextSeq 500/550 platform, the Illumina NovaSeq 6000 platform, or the NextSeq 1000/2000 platform for sequencing.

Various steps of the methods of the invention involve oligonucleotide generation and/or amplification. Such reactions, as well as the sequencing reaction, may comprise the use of primer sequences.

Accordingly, the present invention relates to an oligonucleotide capable of specifically amplifying the oligonucleotides of the present invention. Accordingly, oligonucleotides within the meaning of the invention may be capable of serving as a starting point for amplification, i.e. may be capable of serving as primers. Such oligonucleotide may comprise oligoribo- or desoxyribonucleotides which are complementary to a region of one of the strands of an oligonucleotide. According to the present invention, a person skilled in the art would readily understand that the term “primer” may also refer to a pair of primers that are with respect to a complementary region of an oligonucleotide directed in the opposite direction towards each other to enable, for example, amplification by polymerase chain reaction (PCR). Purification of the primer(s) is generally envisaged, prior to its/their use in the method of the present invention. Such purification steps can comprise HPLC (high performance liquid chromatography) or PAGE (polyacrylamide gel-electrophoresis), and are known to the person skilled in the art.

When used in the context of primers, the term “specifically” means that preferably or exclusively the desired oligonucleotides as described herein are amplified. Thus, a primer according to the invention is preferably a primer, which binds to a region of an oligonucleotide which is unique for this molecule. In connection with a pair of primers, according to the invention, it is possible that one of the primers of the pair is specific in the above described meaning or both of the primers of the pair are specific.

The 3′-OH end of a primer is used by a polymerase to be extended by successive incorporation of nucleotides. Preferably, the primer or pair of primers of the present invention are used for amplification reactions on template oligonucleotides. The term “template” refers to oligonucleotides or fragments thereof of any source or composition, that comprise a target oligonucleotide sequence. It is known that the length of a primer results from different parameters (Gillam, Gene 8 (1979), 81-97; Innis, PCR Protocols: A guide to methods and applications, Academic Press, San Diego, USA (1990)). Preferably, the primer should only hybridize or bind to a specific region of a target oligonucleotide. The length of a primer that statistically hybridizes only to one region of a target nucleotide sequence can be calculated by the following formula: (¼)^{x}(whereby x is the length of the primer). However, it is known that a primer exactly matching to a complementary template strand must be at least 9 base pairs in length, otherwise no stable-double strand can be generated (Goulian, Biochemistry 12 (1973), 2893-2901). It is also envisaged that computer-based algorithms can be used to design primers capable of amplifying DNA. It is also envisaged that the primer or pair of primers is labeled. The label may, for example, be a radioactive label, such as 32P, 33P or 35S. In a preferred embodiment of the invention, the label is a non-radioactive label, for example, digoxigenin, biotin and fluorescence dye or dyes.

The invention furthermore relates to the use of a microfluidic system, in particular a microfluidic droplet generator, in the methods of the invention. The microfluidic system may be in particular used to generate (microfluidic) droplets or to deliver material into a well- or chamber-based device, like into microfluidic well-based device Such devices are known in the art and are, inter alia, based on integrated fluidic circuit technologies. An example of such a provider for such devices is Fluidigm Corporation/U.S.A. Accordingly, the generation of (microfluidic) droplets or the delivery of material into a well- or chamber-based device may also be part of the methods of the present invention. An exemplary droplet generator is the Chromium™ Controller provided by 10× Genomics (Pleasanton, CA). Further examples include Drop-seq and inDrop platforms. Moreover, the invention can be used to boost the throughput of sub-nanoliter well based platforms such as CytoSeq (Fan et al., 2015), Seq-Well (Gierahn et al., 2017), Microwell-Seq (Han et al, 2018) or microfluidic systems with built-in reaction chambers. A compatible commercial version is the above mentioned BD Rhapsody™ system on which the methods of the invention can be shown to provide surprising results.

The methods of the invention may further comprise an additional layer of multiplexing by cell hashing.

As provided herein, the methods of the present invention may be used in synthetic biology. For example, the methods of the present invention may be used with a gene panel readout (e.g. a few 10s to 100s of specifically assayed genes instead of a whole-transcriptome readout). As such, provided is a device that uses single-cell RNA-seq, the methods of the present invention, to replace flow cytometry as a key diagnostic assay (especially when combined with barcoded antibodies and/or TCR/BCR immune repertoire profiling) in cancer, immune disorders, and many other diseases. In a further envisaged embodiment, the methods of the present invention are combined with guide-RNA enrichment for massive-scale CRISPR single-cell sequencing (CROP-seq, Perturb-seq, etc. —using CRISPR knockout, CRISPR activation, CRISPR knockdown, CRISPR knock-in of natural or synthetic sequences, CRISPR epigenome editing, saturation mutagenesis or similar assays for the perturbation step) with hypothesis-driven gene set/pathway readout.

Further provided are the methods of the present invention combined with ChIPmentation as described in WO 2017/025594 as a separate assay based on the same technology (e.g. for single-cell epigenome profiling) or combined with guide-RNA enrichment (e.g. for epigenome-based CROP-seq screens).

The methods of the invention also provided for use in drug discovery, drug screening, testing of compounds and/or target validation. As such, the methods of the invention are able to derive, inter alia, relevant screening signatures directly from the transcriptome of control cells, so that no prior knowledge about the mechanism of action of a drug and/or test compound is required. Moreover, the single-cell resolution of the methods of the invention allows to assess the effect of a drug/test compound to be screened on different cell types in a complex mixture (for instance, but not limited to, PBMCs), or on a mixture of cells from distinct donors.

Accordingly, provided herein, is a method for identifying and/or screening a test compound able to alter the transcriptome of a cell, the method comprising the steps of:
(a) contacting cells and/or nuclei which comprise a first oligonucleotide comprising RNA with one or more test compound(s) to be identified and/or screened;
(b) permeabilizing said cells and/or nuclei which comprise said first oligonucleotide comprising RNA;
(c) combining said cells and/or nuclei of (b) with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide;
(d) reversely transcribing the first oligonucleotide in said cells/nuclei to obtain an elongated second oligonucleotide;
(e) combining said cells and/or nuclei obtained in step (d) with a microbead-bound third oligonucleotide in a second reaction compartment, wherein the third oligonucleotide comprises
   (i) a first sequence corresponding to a fourth sequence comprised in the second oligonucleotide used in step (c); or
   (ii) a first sequence complementary to a first sequence of a fourth oligonucleotide, wherein the fourth oligonucleotide further comprises a second sequence at least partially complementary to the third sequence of the second oligonucleotide;
wherein for (i), the method further comprises a step of second strand DNA synthesis subsequent to step (d) and prior to step (e) and wherein for (ii) the method further comprises a step of DNA ligation;
and wherein the third oligonucleotide further comprises a second sequence comprising an indexing sequence and a third sequence comprising a primer binding site;
(f) amplifying the DNA oligonucleotides obtained in step (e);
(g) sequencing of amplified DNA oligonucleotides; and
(h) identifying the test compound(s) as compound(s) able to alter the transcriptome of a cell if the sequenced DNA oligonucleotides differ from the sequenced DNA oligonucleotides obtained by the method without step (a).

In the above method, said “first oligonucleotide comprising RNA” as comprised in said cells and/or nuclei may be a naturally occurring RNA but may also be a synthetically synthesized, chimeric and/or artificial RNA construct, like an guide RNA and/or shRNAs as employed in the CRISPR technology, a viral or viral derived nucleic acid as, inter alia, used for gene transfers, etc. Non-limiting examples of such “first oligonucleotides comprising RNA” include: the cell's naturally occurring transcriptome, other naturally occurring or artificial small RNAs, such as tRNA, snRNA, snoRNA, micro-RNA, rRNA, synthetic biology tools such as riboswitches and RNA aptamers, combinations of RNAs as employed in CRISPR technologies, like combinations of guide RNAs or shRNAs in the same cell e.g. (co-essentiality, combined action), synthetic genes and synthetic mutagenized gene libraries, RNA barcodes, e.g. to mark sample of origin, spatial location, treatment, transgenes, RNA barcodes from lineage tracing experiments, RNA barcodes connected to antibodies expressed in a given cell, RNA barcodes marking the location on a tissue slice, RNA barcodes marking cell-cell interactions, RNA barcodes that label (cell surface) proteins (intracellular proteins or modified amino acid residues (for example via antibodies), RNA barcodes used as synthetic readers of biological processes, viral RNA, for example to assess the infection state of a cell, immune receptors such as chimeric antigen receptors or T cell receptors, (synthetic) transcription factors, (synthetic) homing receptors, etc.

As with all means and methods as provided in the present invention, also this method for identifying and/or screening a test compound able to alter the transcriptome of a cell as provided herein and comprising the step “permeabilized cells and/or nuclei comprising a first oligonucleotide comprising RNA” (herein above in step (b)), may comprise an additional, optional step wherein said cells/nuclei are fixed. Fixation of cells/nuclei are known in the art and comprise, inter alia but preferably, chemical cross-linking (like, e.g, with formaldehyde or alcohols, like methanol). This fixing step may comprise the fixing of the RNAs to be analyzed in context of the herein provided methods in and on their cellular context, for example, on structural components of the cells/nuclei etc. Such an optional fixing step has also the advantage that said cells/nuclei may be preserved/conserved and/or that these fixed cells/nuclei may be employed/analyzed at a later point of time. Such a preservation/conservation may comprise freezing of said permeabilized and fixed cells/nuclei.

Said one or more test compound(s) to be screened/validated/identified and/or used in the method as recited above may be selected from the group of small molecules, large molecules, RNA, DNA and other compounds, including chemical compounds and/or pharmaceuticals. But also biological material and/or pathogens may be the “test compound” to be screened/identified and/or used in the methods of the present invention. Such biological material and/or pathogens may comprise bacteria, viruses, fungi and/or other biological material, like multicellular pathogens, like nematodes, jellyfish, etc. The term “biological material and/or pathogens” also comprises parts of said materials/pathogens, like, inter alia, proteins, peptides, nucleic acids, mixtures of such materials/pathogens, extracts etc. Said test compound(s) may also be a compound or group of compounds resulting in genetic perturbations, such as CRISPR modifications and/or edits in the genome of the cell and/or nuclei.

Further examples of the “test compound” to be employed in the methods of the present invention comprise, but are not limited to, compounds that lead to a status modification and/or change in a given cell, like a change in differentiation status or leading to apoptosis. The “test compound” may also be an mRNA to be introduced in the cells/nuclei, a plasmid, a viral vector etc. Such compounds may also be used, inter alia, for gene transfer. Such “coding” nucleic acids and/or gene transfer shuttles may encode, without being limiting for transcription factors, epigenetic regulators, kinases, homing receptors to control the localization of cells within an organism or tissue, immune co-stimulatory domains (such as 41BB, CD27, CD28, OX40, CD2, or CD40L), or immune co-inhibitory domains (such as BTLA, CTLA4, LAG3, LAIR1, PD-1, TIGIT or TIM3). Also constituents of receptor/ligand systems (or isolated parts thereof, like extracellular domains and/or soluble parts) may be employed as “test compounds”. Non-limiting examples of such receptor/ligand systems include, inter alia, molecules of signaling pathways and/or immunomodulation pathways, like the PD-1/PD-L1/PD-L2 system(s), or CD40/CD40L system(s), B7-1, B7-2, etc.

As is evident from the current description and in context of this invention, the examples for “test compounds” as provided herein above are not limited to the above discussed “method for identifying and/or screening a test compound able to alter the transcriptome of a cell”. These “test compounds” may be also employed in the general method for sequencing oligonucleotides provided herein, i.e. in the inventive scifi-RNA-seq method and variations thereof.

The methods of the invention may also combine various steps as also illustrated herein and in the appended examples. Particularly preferred are versions of the invention, like EXT-TN5 (Example 3), LIG-TS (Example 4), EXT-RP (Example 5), LIG-RP (Example 6) and/or EXT-TS (Example 7). Each of these versions of the inventive mean and method are particularly useful to increase the number of uniquely labeled cells and thus the throughput as compared to existing methods.

Thus, in a particular embodiment, the present invention relates to a method for sequencing oligonucleotides comprising RNA (EXT-TN5), the method comprising the steps of:
(a) providing permeabilized cells and/or nuclei comprising a first oligonucleotide comprising RNA;
(b) combining said cells and/or nuclei of (a) with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide;
(c) reversely transcribing the first oligonucleotide in said cells/nuclei to obtain an elongated second oligonucleotide;
(d) synthesizing a second DNA strand and introducing untemplated nucleotides at the 5′-end of the synthesized second strand DNA using a transposase enzyme, in particular Tn5 transposase;
(e) combining said cells and/or nuclei obtained in step (d) with a microbead-bound third oligonucleotide in a second reaction compartment, wherein the third oligonucleotide comprises a first sequence corresponding to a fourth sequence comprised in the second oligonucleotide used in step (b) and wherein the third oligonucleotide further comprises a second sequence comprising an indexing sequence and a third sequence comprising a primer binding site;
(f) amplifying the DNA oligonucleotides obtained in step (e); and
(g) sequencing of amplified DNA oligonucleotides.

In a particular embodiment, the present invention relates to a method for sequencing oligonucleotides comprising RNA (LIG-TS), the method comprising the steps of:
(a) providing permeabilized cells and/or nuclei comprising a first oligonucleotide comprising RNA;
(b) combining said cells and/or nuclei of (a) with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide;
(c) reversely transcribing the first oligonucleotide in said cells/nuclei to obtain an elongated second oligonucleotide, wherein untemplated nucleotides are added to the 3′-end of the second oligonucleotide;
(d) combining said cells and/or nuclei obtained in step (c) with a microbead-bound third oligonucleotide in a second reaction compartment, wherein the third oligonucleotide comprises a first sequence complementary to a first sequence of a fourth oligonucleotide, wherein the fourth oligonucleotide further comprises a second sequence at least partially complementary to the third sequence of the second oligonucleotide; and wherein the third oligonucleotide further comprises a second sequence comprising an indexing sequence and a third sequence comprising a primer binding site;
(e) ligating second and third oligonucleotide using a DNA ligase, preferably a thermostable DNA ligase;
(f) extending ligated oligonucleotide by adding a primer comprising RNA nucleotides and adding a reverse transcriptase enzyme;
(g) amplifying the DNA oligonucleotides obtained in step (f); and
(h) sequencing of amplified DNA oligonucleotides.

In a particular embodiment, the present invention relates to a method for sequencing oligonucleotides comprising RNA (EXT-RP), the method comprising the steps of:
(a) providing permeabilized cells and/or nuclei comprising a first oligonucleotide comprising RNA;
(b) combining said cells and/or nuclei of (a) with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide;
(c) reversely transcribing the first oligonucleotide in said cells/nuclei to obtain an elongated second oligonucleotide;
(d) synthesizing second DNA strand;
(e) combining said cells and/or nuclei obtained in step (d) with a microbead-bound third oligonucleotide in a second reaction compartment, wherein the third oligonucleotide comprises a first sequence corresponding to a fourth sequence comprised in the second oligonucleotide used in step (b); and wherein the third oligonucleotide further comprises a second sequence comprising an indexing sequence and a third sequence comprising a primer binding site;
(f) adding a primer comprising random nucleotides for linear extension;
(g) amplifying the DNA oligonucleotides obtained in step (f); and
(h) sequencing of amplified DNA oligonucleotides.

In a particular embodiment, the present invention relates to a method for sequencing oligonucleotides comprising RNA (LIG-RP), the method comprising the steps of:
(a) providing permeabilized cells and/or nuclei comprising a first oligonucleotide comprising RNA;
(b) combining said cells and/or nuclei of (a) with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide;
(c) reversely transcribing the first oligonucleotide in said cells/nuclei to obtain an elongated second oligonucleotide;
(e) combining said cells and/or nuclei obtained in step (d) with a microbead-bound third oligonucleotide in a second reaction compartment, wherein the third oligonucleotide comprises a first sequence complementary to a first sequence of a fourth oligonucleotide, wherein the fourth oligonucleotide further comprises a second sequence at least partially complementary to the third sequence of the second oligonucleotide; and wherein the third oligonucleotide further comprises a second sequence comprising an indexing sequence and a third sequence comprising a primer binding site;
(f) ligating second and third oligonucleotide using a DNA ligase, preferably a thermostable DNA ligase;
(g) adding a primer comprising random nucleotides for linear extension;
(h) amplifying the DNA oligonucleotides obtained in step (g); and
(i) sequencing of amplified DNA oligonucleotides.

In a particular embodiment, the present invention relates to a method for sequencing oligonucleotides comprising RNA (EXT-TS), the method comprising the steps of:
(a) providing permeabilized cells and/or nuclei comprising a first oligonucleotide comprising RNA;
(b) combining said cells and/or nuclei of (a) with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide;
(c) reversely transcribing the first oligonucleotide in said cells/nuclei to obtain an elongated second oligonucleotide, wherein untemplated nucleotides are added to the 3′-end of the second oligonucleotide, and
wherein a primer comprising RNA nucleotides complementary to the added untemplated nucleotides is added for extension;
(d) combining said cells and/or nuclei obtained in step (d) with a microbead-bound third oligonucleotide in a second reaction compartment, wherein the third oligonucleotide comprises a first sequence corresponding to a fourth sequence comprised in the second oligonucleotide used in step (b); and wherein the third oligonucleotide further comprises a second sequence comprising an indexing sequence and a third sequence comprising a primer binding site;
(e) amplifying the DNA oligonucleotides obtained in step (d); and
(f) sequencing of amplified DNA oligonucleotides.

The above recited versions of the present invention, like EXT-TN5 (also illustrated in appended Example 3), LIG-TS (also illustrated in appended Example 4), EXT-RP (also illustrated in appended Example 5), LIG-RP (also illustrated in appended Example 6) and EXT-TS (also illustrated in appended Example 7) may also, optionally, comprise an additional step wherein the permeabilized cells and/or nuclei comprising a first oligonucleotide comprising RNA are fixed before the following steps are carried out. Accordingly, if desired, an optional fixation step may be carried out after step (a) as recited for the scifi-RNA-seq method and its variants as provided herein above.

The present invention also relates to kits, in particular research kits. The kits of the present invention comprise the second oligonucleotide of the present invention, preferably together with instructions regarding the use of the methods of the invention. The kits of the invention may further comprise a hyperactive, preferably also oligonucleotide loaded, tranposase and/or reagents for second strand synthesis. The kits of the invention may also comprise the transposase enzyme in a ready-to-use form. Further comprised may be one or more of the other oligonucleotides used in the present invention, for example the fourth oligonucleotide and/or the thermostable ligase. The kits of the invention may be used inter alia in research applications such as the sequencing of RNA molecules.

In a particularly preferred embodiment of the present invention, the kits (to be prepared in context) of this invention or the methods and uses of the invention may further comprise or be provided with (an) instruction manual(s). For example, said instruction manual(s) may guide the skilled person (how) to employ the kit of the invention in the diagnostic uses provided herein and in accordance with the present invention. Particularly, said instruction manual(s) may comprise guidance to use or apply the herein provided methods or uses.

The kit (to be prepared in context) of this invention may further comprise substances/chemicals and/or equipment suitable/required for carrying out the methods and uses of this invention. For example, such substances/chemicals and/or equipment are solvents, diluents and/or buffers for stabilizing and/or storing and/or enabling enzymatic reactions or terminating enzymatic reactions, (a) compound(s) required for the uses provided herein, like stabilizing and/or storing the chemical agent(s) and/or transposase comprised in the kits of the present invention.

Further embodiments are exemplified in the scientific part. The appended figures provide for illustrations of the present invention. Whereas the experimental data in the examples and as illustrated in the appended figures are not considered to be limiting. The technical information comprised therein forms part of this invention.

The invention thus also covers all further features shown in the figures individually, although they may not have been described in the previous or following description. Also, single alternatives of the embodiments described in the figures and the description and single alternatives of features thereof can be disclaimed from the subject matter of the other aspect of the invention.

FIG. 1: Single-Cell Combinatorial Indexing with Fluidic Indexing (Scifi) Combines Pre-Indexing of Entire Transcriptomes with Droplet-Based Single-Cell RNA-Seq

a) Standard droplet-based scRNA-seq using a microfluidic droplet generator is highly inefficient in its use of the droplets. Most droplets contain both a barcoded microbead and the reverse transcription reagents (and are thus fully functional), but never receive a cell; furthermore, the reagents within a droplet are sufficient to barcode more than one cell. b) scifi-RNA-seq unlocks the full potential of microfluidic droplet generators. Prior to the microfluidic run, entire transcriptomes are pre-indexed by reverse transcription inside permeabilized cells or nuclei (round1 barcodes indicated by letters A to F). The differentially barcoded pool of cells/nuclei is loaded at fill rates e.g. around 10 per droplet. Cells inside the same emulsion droplet are labelled with an identical microfluidic (round2) barcode, but can still be distinguished via their transcriptome (round1) index.

FIG. 2: scifi-RNA-seq based on linear extension and a custom Tn5 transposome (version EXT-TN5) Inside intact cells or nuclei, mRNA is reverse transcribed. Second strand synthesis is performed by nicking the RNA template, extending with a polymerase and closing nicks with a ligase. Double stranded cDNA is tagmented with a custom i7-only Tn5 transposome. In a second reaction compartment, the round2 index is introduced by linear extension with a polymerase. The final library is enriched by PCR and sequenced.

FIG. 3: scifi-RNA-seq based on linear extension and random priming (EXT-RP) Inside intact cells or nuclei, mRNA is reverse transcribed. Second strand synthesis is performed by nicking the RNA template, extending with a polymerase and closing nicks with a ligase. In a second reaction compartment, the round2 index is introduced by linear extension with a polymerase. The P7 sequencing adapter is introduced by random priming. The final library is enriched by PCR and sequenced.

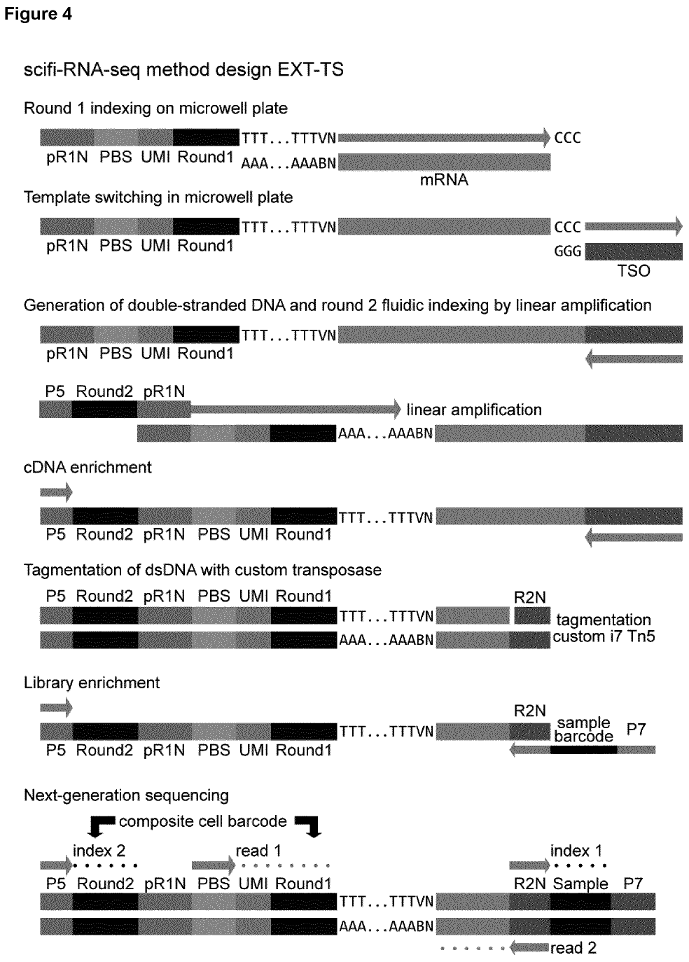

FIG. 4: scifi-RNA-seq based on linear extension and template switching (EXT-TS) Inside intact cells or nuclei, mRNA is reverse transcribed under conditions that allow the addition of untemplated C bases. Template switching extends the cDNA molecule on the 3′ end. In a second reaction compartment, double-stranded cDNA is generated by extension of a TSO enrichment primer, and the round2 barcode is introduced by extension with a polymerase. Afterwards, the cDNA library is enriched by PCR and can be further processed by established methods such as a commercially available or custom transposome, fragmentation followed by adapter ligation, or random priming. The final library is enriched by PCR and sequenced.

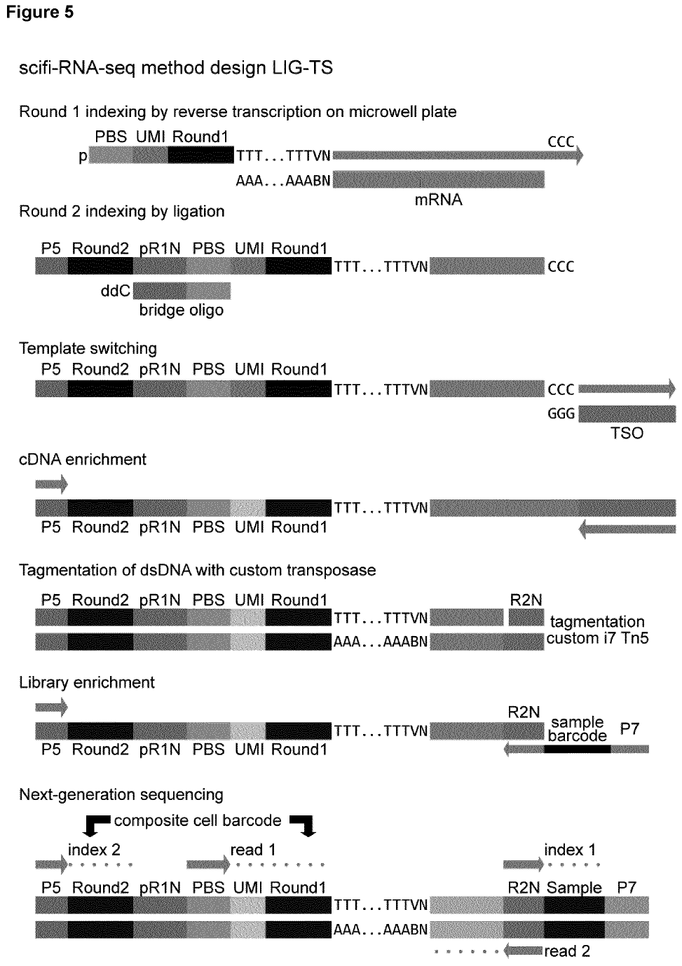

FIG. 5: scifi-RNA-seq based on thermocycling ligation and template switching (version LIG-TS) Inside intact cells or nuclei, mRNA is reverse transcribed under conditions that allow the addition of untemplated C bases, with a 5′-phosphorylated reverse transcription primer. In a second reaction compartment, the round2 barcode is introduced by ligating an indexed oligonucleotide with a ligase, preferably a thermostable ligase. This ligation requires a compatible bridge oligo, preferably blocked on the 3′-end. Template switching then extends the cDNA molecule on the 3′ end. Afterwards, the cDNA library is enriched by PCR and can be further processed by established methods such as a commercially available or custom transposome, fragmentation followed by adapter ligation, or random priming. The final library is enriched by PCR and sequenced.

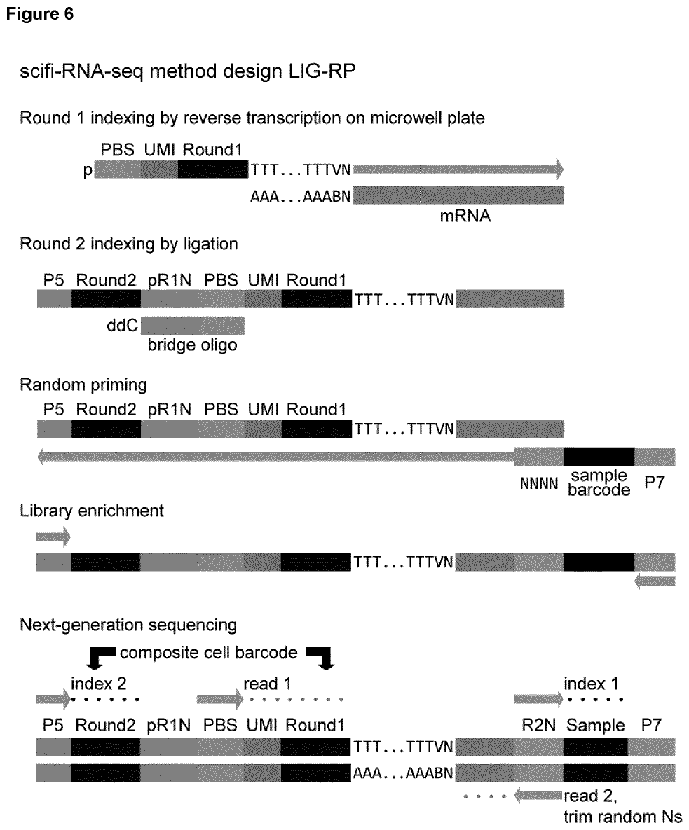

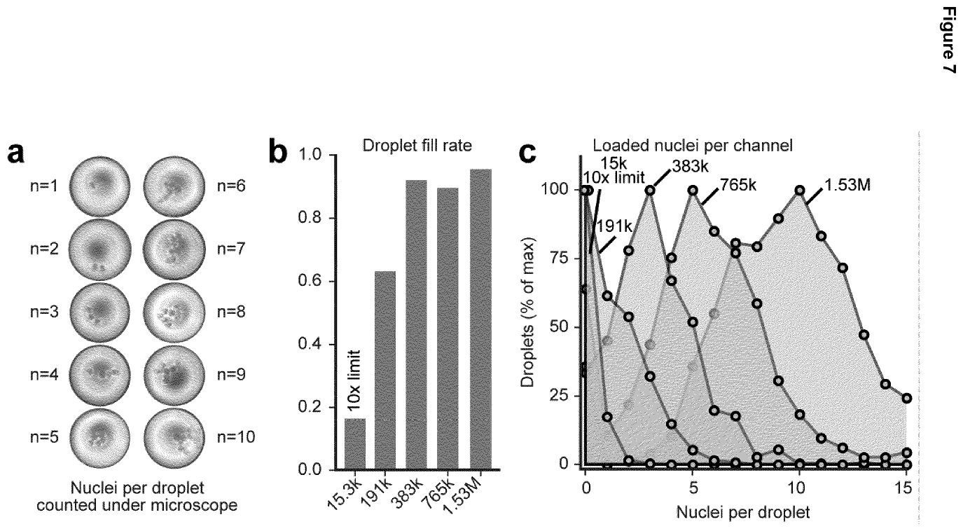

FIG. 6: scifi-RNA-seq based on thermocycling ligation and random priming (version LIG-RP) Inside intact cells or nuclei, mRNA is reverse transcribed with a 5′-phosphorylated reverse transcription primer. In a second reaction compartment, the round2 barcode is introduced by ligating an indexed oligonucleotide with a ligase, preferably a thermostable ligase. This ligation requires a compatible bridge oligo, preferably blocked at the 3′-end. Random priming then introduces the P7 sequencing adapter at the 3′ end. Afterwards, the cDNA library is enriched by PCR and can be further processed by established methods such as a commercially available or custom transposome, fragmentation followed by adapter ligation, or random priming. The final library is enriched by PCR and sequenced.

FIG. 7: a) By omitting the lysis reagents, intact nuclei can be imaged inside emulsion droplets, confirming the feasibility of overloading microfluidic droplet generators. Representative droplets containing between 1 and 10 nuclei are shown. b) Overloading boosts the percentage of droplets filled with nuclei from 16.4% (10× Genomics maximum) to 95.5% (100-fold overloading using 1.53 million nuclei per channel). c) Overloading causes the average number of nuclei per droplet to increase in a controlled fashion while maintaining the desired random loading distribution.

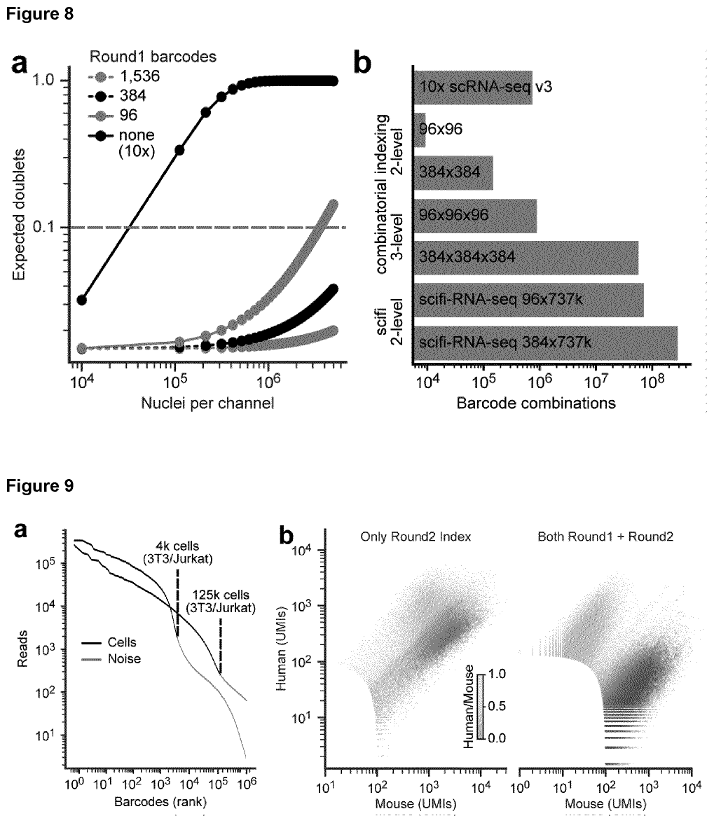

FIG. 8: a) Expected doublet rate as a function of the cell/nuclei loading concentration per channel for defined sets of round1 barcodes. The cell/nuclei fill rate was modelled as a zero-inflated Poisson distribution. b) Due to the high number of microfluidic round2 barcodes, 2-level scifi exceeds the barcode combinations of 3-level combinatorial indexing.

FIG. 9: a) Cells/Nuclei pre-processed with the scifi-RNA-seq protocol are stable in a microfluidic run. Plotting barcode rank versus sequenced reads on logarithmic scales identifies a characteristic inflection point that separates cells/nuclei from noise. The results indicate that scifi-RNA-seq can recover input cells/nuclei with high efficiency. b) The round1 transcriptome index can deconvolute multiple cells/nuclei per droplet into the respective single-cell transcriptomes. 125,000 nuclei/cells of a 1:1 mixture of human (Jurkat) and mouse (3T3) cells and nuclei was processed and demultiplexed based on the microfluidic round2 barcode only (left plot), or based on the combination of round1 and round2 barcodes (right plot).

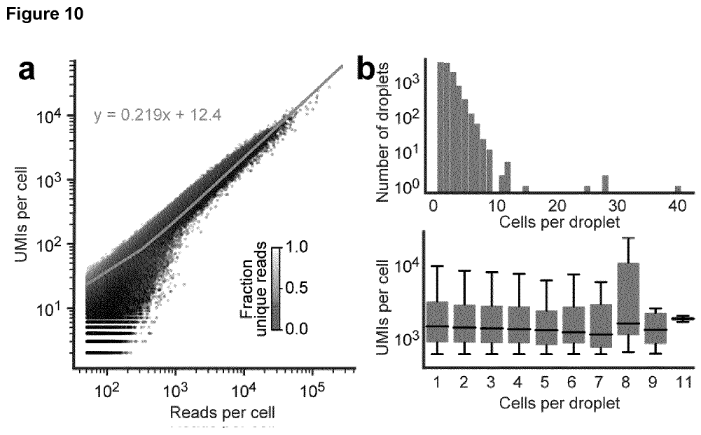

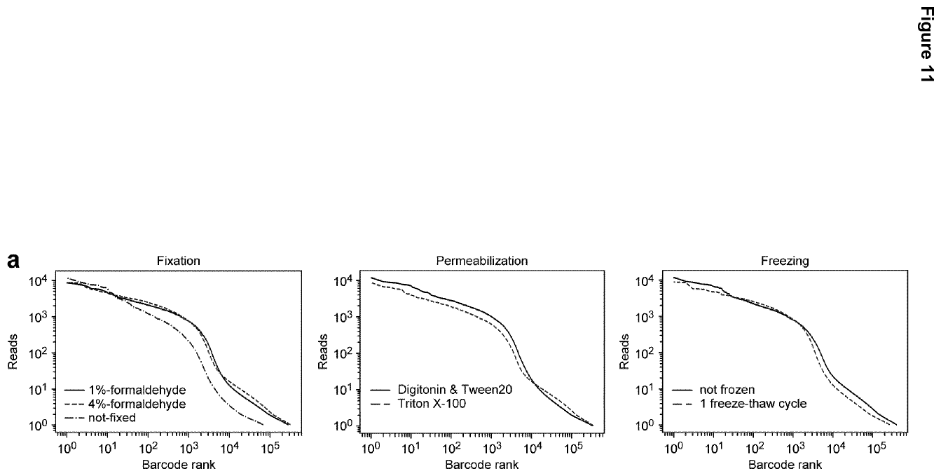

FIG. 10: a) Performance plot showing unique molecular identifiers (UMIs) per cell/nucleus as a function of the sequencing coverage. The fraction of unique reads is shown as a gradient. b) UMIs per cell/nucleus are plotted against the number of cells/nuclei contained in their respective droplet, indicating that there is no decrease in library complexity for high numbers of cells/nuclei per droplet.

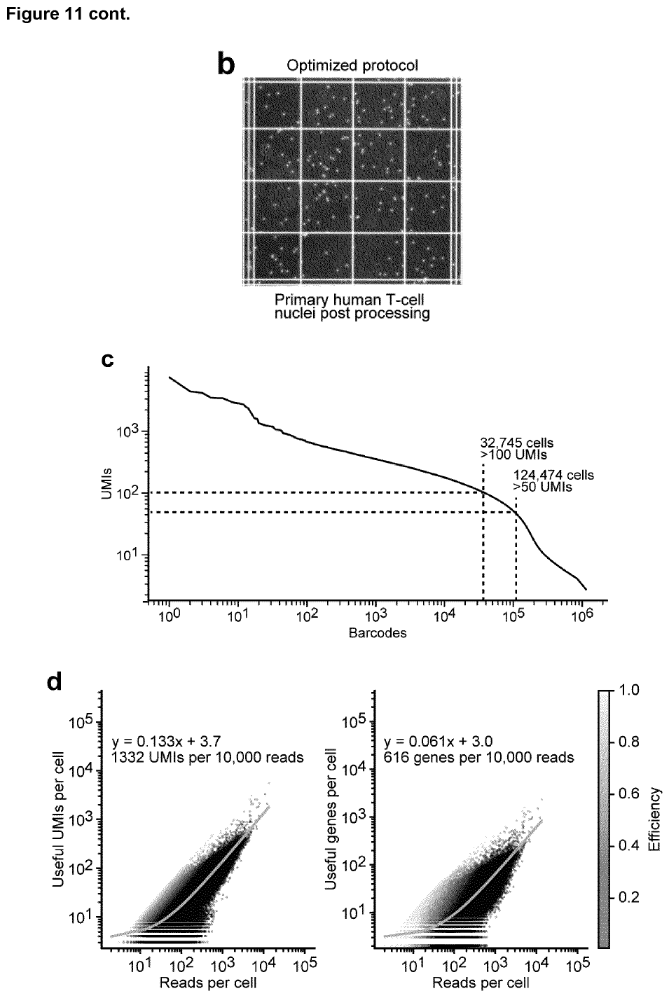

FIG. 11: a) Optimization of fixation and permeabilization conditions for the processing of human primary T-cells. One freeze-thaw cycle did not have a negative impact on the data quality; sampling and library preparation can thus be done on separate days or in separate labs, which adds to the usability and flexibility of the assay. b) Primary human T-cell nuclei after reverse transcription and second strand synthesis, visualized in a Fuchs Rosenthal counting chamber. An optimized protocol using fixation with 4% formaldehyde, freezing at −80° C. and permeabilization with Digitonin and Tween-20 was used to stabilize nuclei. c) Detected cell barcodes (x-axis) are ranked according to the sequenced reads per barcode (y-axis). The characteristic inflection point indicates that roughly 250,000 cells are contained in the dataset. At a modest sequencing coverage, 32,745 cells had over 100 UMIs, and 124,474 cells had over 50 UMIs. d) Our human primary T-cell dataset contains complex transcriptomic signatures. 10,000 sequenced reads correspond to 1,332 UMIs and 616 genes. Both plots are not saturated, and deeper sequencing would recover many more UMIs per cell.

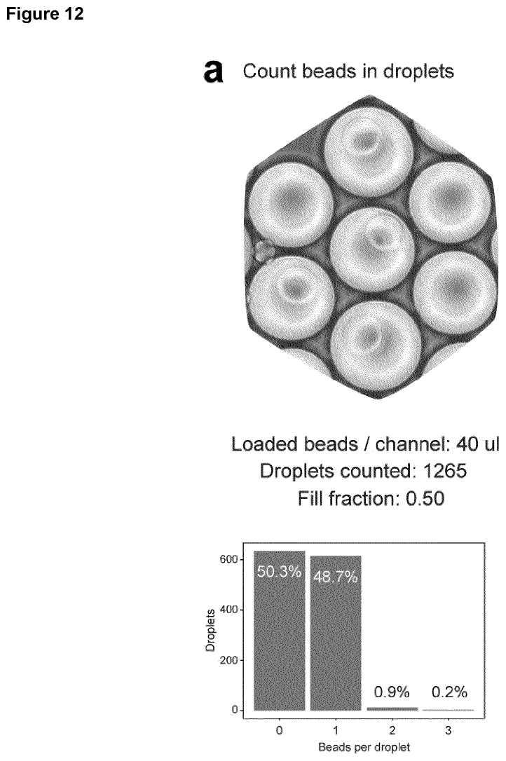

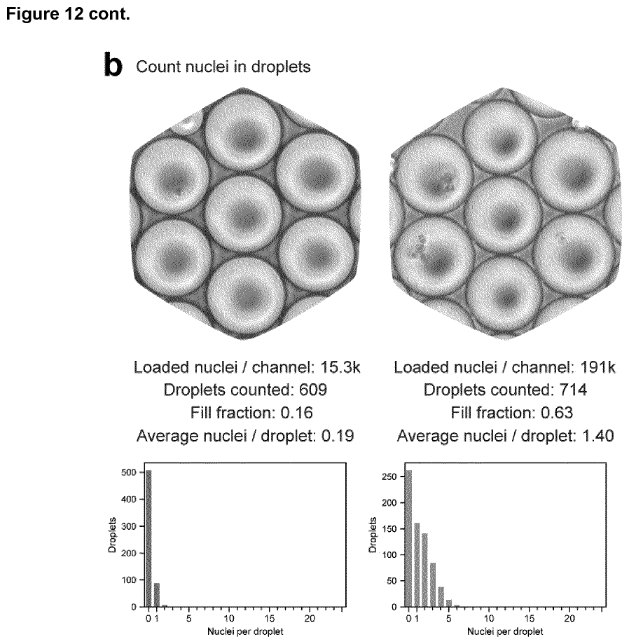

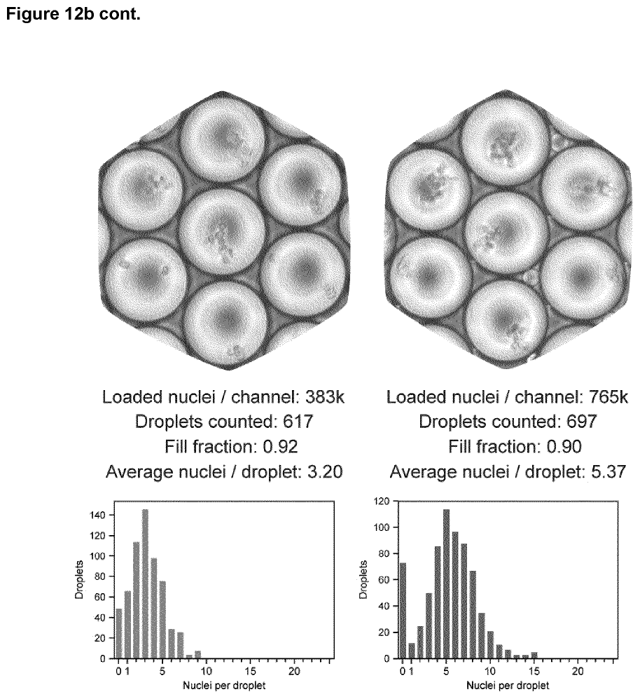

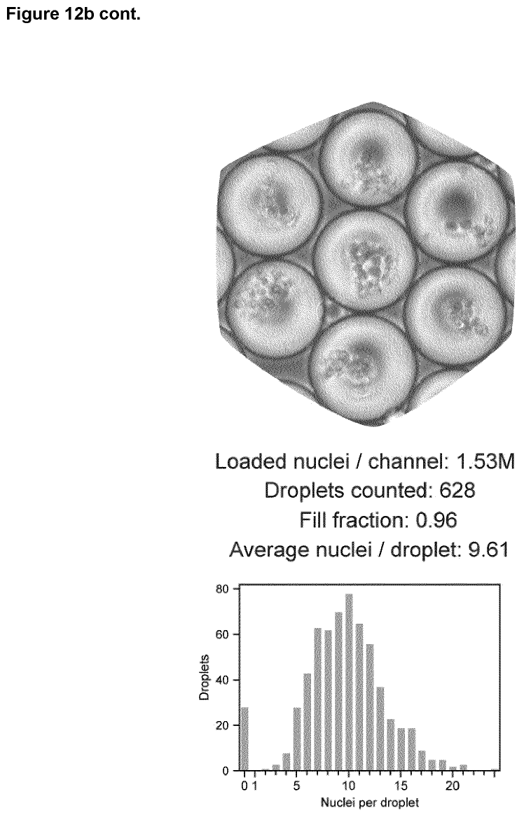

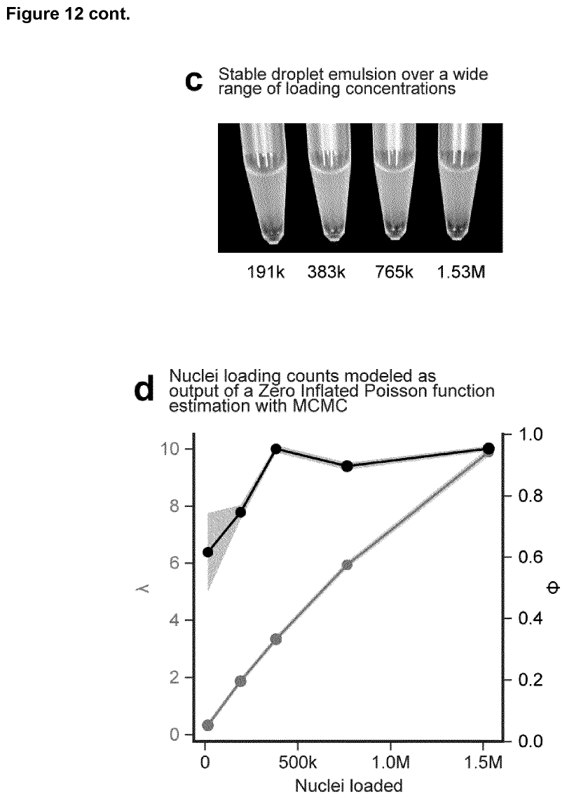

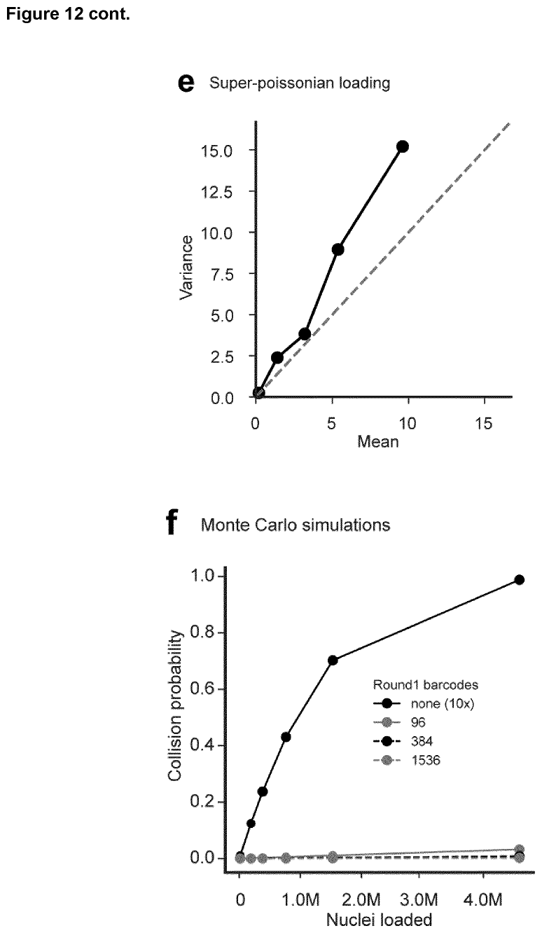

FIG. 12: a) By substituting the nuclei suspension with 1× Nuclei Buffer and omitting Reducing Agent B, intact gel beads could be visualized inside the emulsion droplets. Bead fill rates based on 1,265 evaluated droplet images are shown. b) By omitting the lysis reagents, intact nuclei could be imaged using a standard microscope. For droplets in the correct focal plane, this allows the exact counting of nuclei per droplet. Results for loading concentrations of 15,300, 191,000, 383,000, 765,000, and 1,530,000 cells/nuclei per channel are summarized as histograms. c) Despite substantial overloading of the microfluidic device, we obtained stable droplet emulsions for all tested conditions. d) Computational modeling of cells/nuclei loading as a zero-inflated Poisson function. e) Nuclei loading displays super-poissonian properties. f) Independent estimation of the cell doublet rates through Monte Carlo simulations of the scifi process.

FIG. 13: a) Enrichment of a primary human T-cell library containing 250,000 cells in seven qPCR reactions. Amplification was monitored based on the SYBR Green signal, and reactions were removed from the thermocycler as soon as they reached saturation (cycle 14). b) Typical size distribution of a final scifi-RNA-seq library. A library made from 250,000 primary human T-cells is shown. c-d) Key metrics from next-generation sequencing runs on the Illumina NextSeq 500 and NovaSeq 6000 platforms. e) Relationship of the percentage of occupied cluster positions and percent or number of pass-filter reads on the Illumina NovaSeq 6000 platform. The type of patterned flow cell (SP, S2) is color-coded. This information is intended to help users find the optimal loading for scifi-RNA-seq libraries. f) NGS performance statistics over key scifi-RNA-seq experiments.

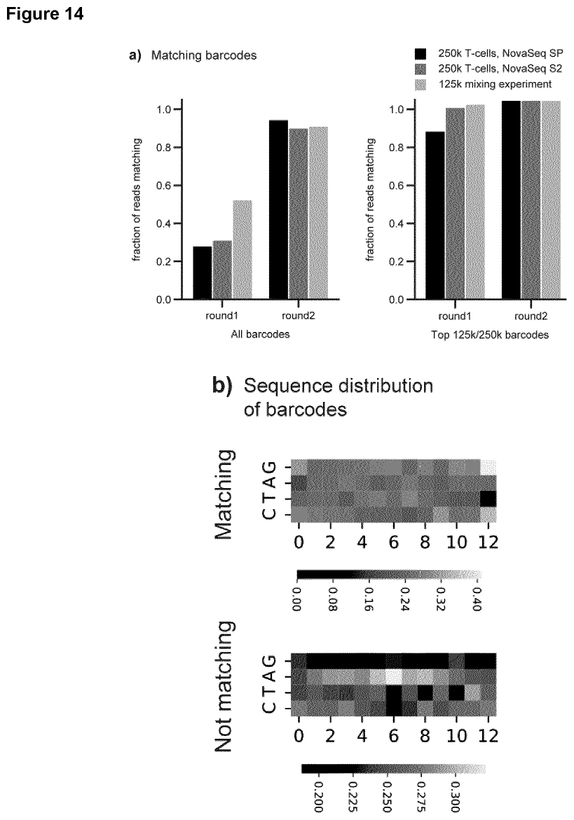

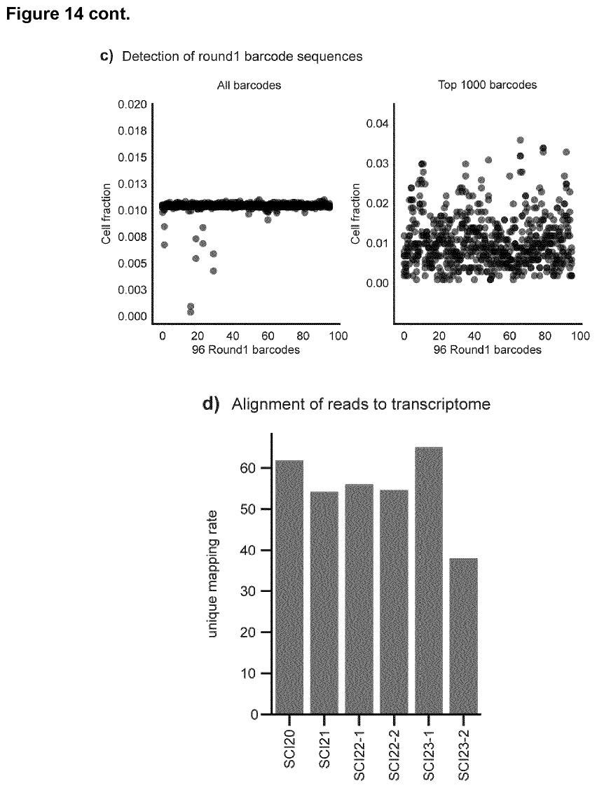

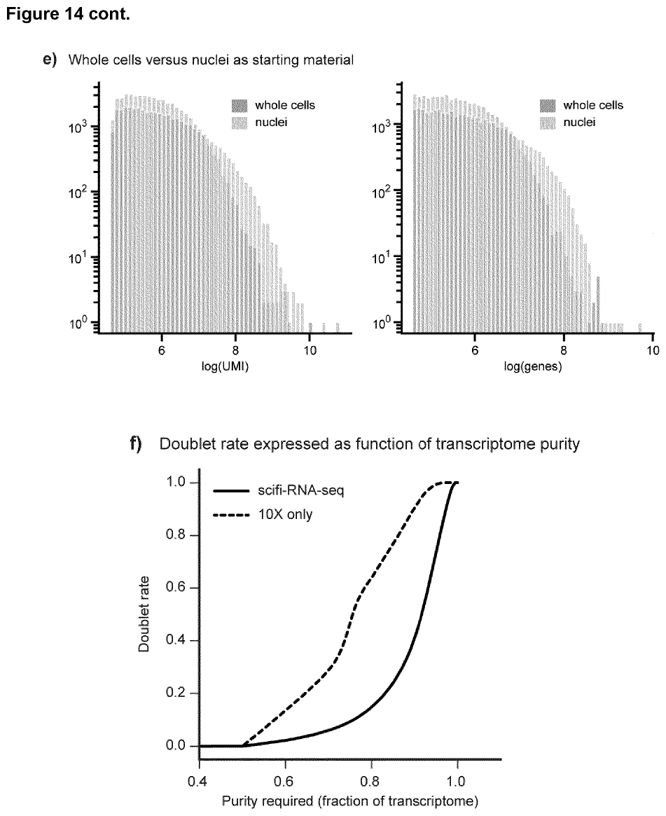

FIG. 14: a) Fraction of total reads with perfect matches to plate-based round1 or microfluidic round2 barcodes. Calculated separately for all detected barcodes (includes background), or barcodes corresponding to real cells (top 125,000 or 250,000 depending on the experiment). b) Matching barcodes show the expected random base distribution for bases 1 to 11, and the fixed V (not T) base at position 12 is detected. Sequences not matching the reference barcodes are biased towards A. c) Abundance of well-specific round1 barcodes is equally distributed over seven scifi-RNA-seq experiments. d) Rates of uniquely aligned reads to the human or mouse transcriptomes for a total of six scifi-RNA-seq runs. e) In a scifi-RNA-seq experiment containing a 1:1 mixture of human (Jurkat) and mouse (3T3) cells and nuclei, nuclei perform slightly better than whole cells. f) Cell doublet rate in a species mixing experiment versus the transcriptome purity threshold.

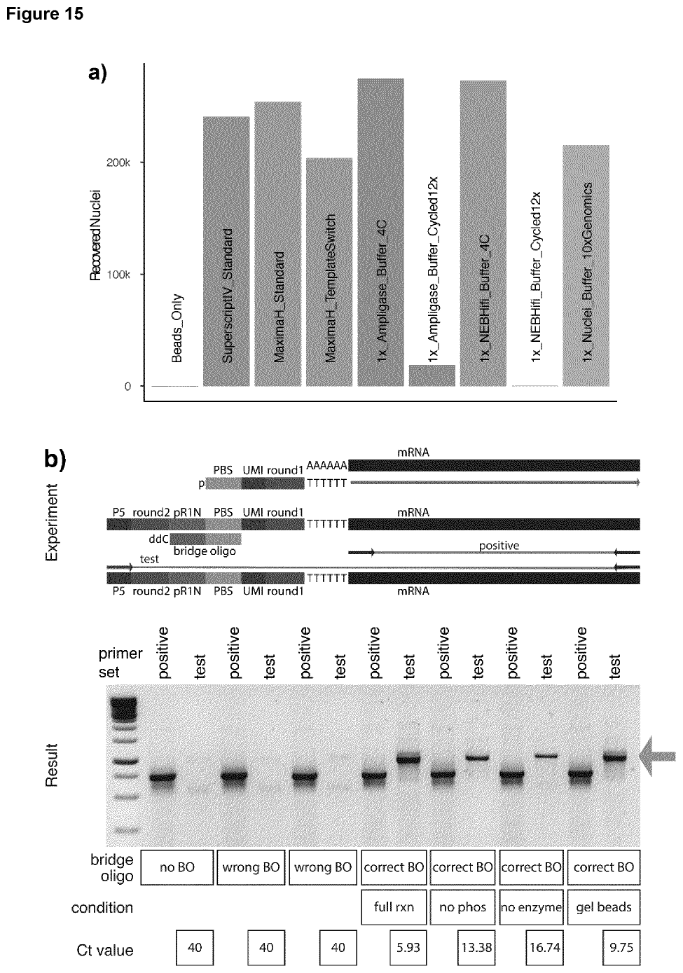

FIG. 15: a) 200,000 nuclei isolated from human Jurkat cells were subjected to reverse transcription reactions (Superscript IV without template switch, Maxima H Minus without template switch and Maxima H Minus with template switch). Afterwards, the number of intact nuclei was quantified by flow cytometry with fluorescent counting beads and visualized in a bar plot. The condition Beads_Only was a negative control reaction containing only counting beads. In a similar experiment, nuclei were instead resuspended in 1× Ampligase buffer (Lucigen), 1× Taq HiFi buffer (NEB) or 1× Nuclei Buffer (10× Genomics) and kept for 1 hour at 4° C. Nuclei were surprisingly stable under those conditions, but were lysed upon thermal cycling of the thermoligation reactions (which is desired to release cellular macromolecules into the emulsion droplets). b) In vitro transcribed, poly-adenylated BFP mRNA was reverse transcribed with 5′-phosphorylated scifi-RNA-seq LIG reverse transcription primer, and subjected to thermoligation with HiFi Taq Ligase. Two amplicons were amplified in a qPCR reaction: ‘positive’ is a positive control for the RT reaction where both primers bind BFP, ‘test’ uses the BFP-FWD primer and the Partial P5 primer, and can amplify only successfully ligated products. Reactions were performed without bridge oligo, with an unmatched bridge oligo, or the correct bridge oligo. When no bridge oligo or an unmatched bridge oligo were used, no ligation product was formed. This shows that the thermoligation reaction is highly specific. Importantly, when the correct bridge oligo was used, the expected ligation product (indicated by the arrow) formed. This was also the case when Single Cell ATAC Gel Beads along with Reducing Agent B (both from 10× Genomics) were used instead of the soluble oligonucleotide substrate. Interestingly, for conditions where the reverse transcription primer had no phosphate group, or where no ligase was used, there was some residual tagged product, probably due to annealing in the qPCR reaction. However, this product was much less abundant (13.38 and 16.74 amplification cycles instead of 5.93 for the full reaction). c) Same experiment as in b), for a wider range of primer binding sites (indrop, dropseq, truseq), thermostable ligases (Taq HiFi, Ampligase), with or without Reducing Agent B. Top: experiment done on poly-adenylated BFP mRNA. Bottom: experiment done on poly-adenylated MS2-p65-HSF1 mRNA. In all cases, the desired ligation product is formed (indicated by the arrow).

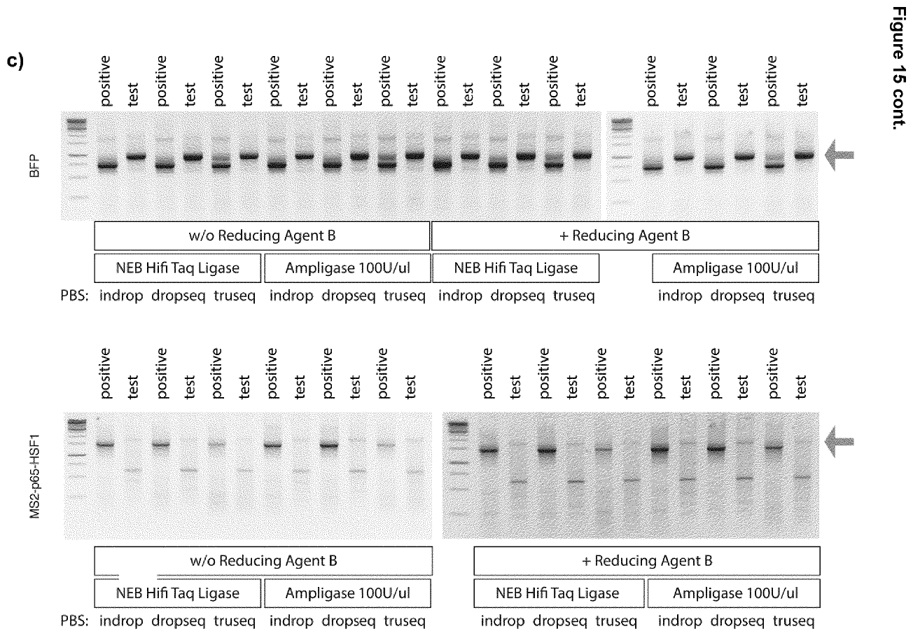

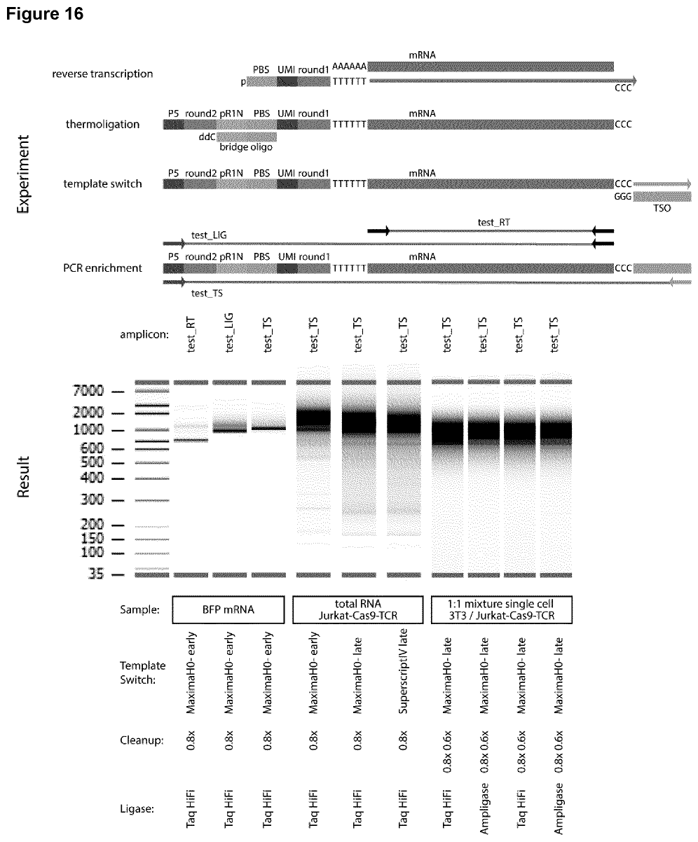

FIG. 16: BFP experiment: Poly-adenylated BFP mRNA was reverse transcribed with 5′-phosphorylated scifi-RNA-seq LIG reverse transcription primer using Maxima H Minus reverse transcriptase that adds untemplated cytosine bases upon reaching the transcript end. The cDNA was subjected to thermoligation with the thermostable ligase Taq HiFi, supplying a tagging oligonucleotide and matching bridge oligo. Afterwards, the 3′-end of the cDNA was tagged by template switching. Three amplicons were enriched by PCR: test_RT is a positive control for the reverse transcription, it uses forward and reverse primers specific for BFP. test_LIG uses the Partial P5 primer and BFP-FWD primer, and can form only upon successful thermoligation. test_TS uses the Partial P5 primer and TSO Enrichment primer and can form only upon successful thermoligation and template switching. Taken together the experiment on BFP mRNA demonstrates that both tagging reactions are successful. Total RNA experiment: The same experiment was performed on total RNA isolated from human Jurkat-Cas9-TCR cells. PCR amplification with Partial P5 and TSO Enrichment primers resulted in a cDNA library. This indicates that both tagging reactions work efficiently when using total RNA as the starting material. Single cell experiment: A similar experiment was performed on a 1:1 mixture of nuclei isolated from human Jurkat-Cas9-TCR cells and mouse 3T3 cells. The reverse transcription reaction was performed on 10,000 intact nuclei per well in a reaction volume of 10 μl. Afterwards, nuclei were pooled, concentrated and resuspended in thermoligation master mix using either Taq HiFi or Ampligase enzymes with their corresponding reaction buffers, and supplying a matching bridge oligonucleotide. Nuclei in reaction mix were then encapsulated into microfluidic droplets on the 10× Genomics Chromium Controller Chip E, along with Single Cell ATAC Gel Beads and Partitioning Oil (10× Genomics). Emulsion droplets were incubated, then the emulsion was broken. The cleaned sample was subjected to template switching and cleaned. cDNA was enriched using Partial P5 and TSO Enrichment primers. This experiment demonstrates that intact nuclei can be used as the starting material and that the thermoligation can be performed inside emulsion droplets.

FIG. 17: a) Design for the enrichment of specific transcripts from scifi-RNA-seq libraries. As an example, the enrichment of CRISPR gRNAs is shown—but the same strategy can be employed to enrich specific transcripts (e.g. immune repertoire of T- and B-cells), entire gene panels, or feature barcodes. In short, the reverse transcription and thermoligation steps are performed as already described. The tagging of the 3′ end via template switching is not required. Instead, PCR enrichment with a transcript-specific primer with a 5′-extension for next-generation sequencing introduces the P7 end of the library. b) Test of four different primers specific to the hU6 promoter in CRISPR gRNA transcripts (e.g. obtained by CROP-seq (Datlinger et al., 2017)). The four primers differ in the length of the P7 extension. This experiment demonstrates that it is possible to introduce the full P7 sequencing adapter in a single-step PCR (primer hU6 full Nextera). c) Enrichment of CRISPR gRNAs using Partial P5 and hU6 full Nextera primers, starting from cDNA obtained in a single-cell scifi-RNA-seq experiment (1:1 mixture of Jurkat-Cas9-TCR and 3T3 cells).

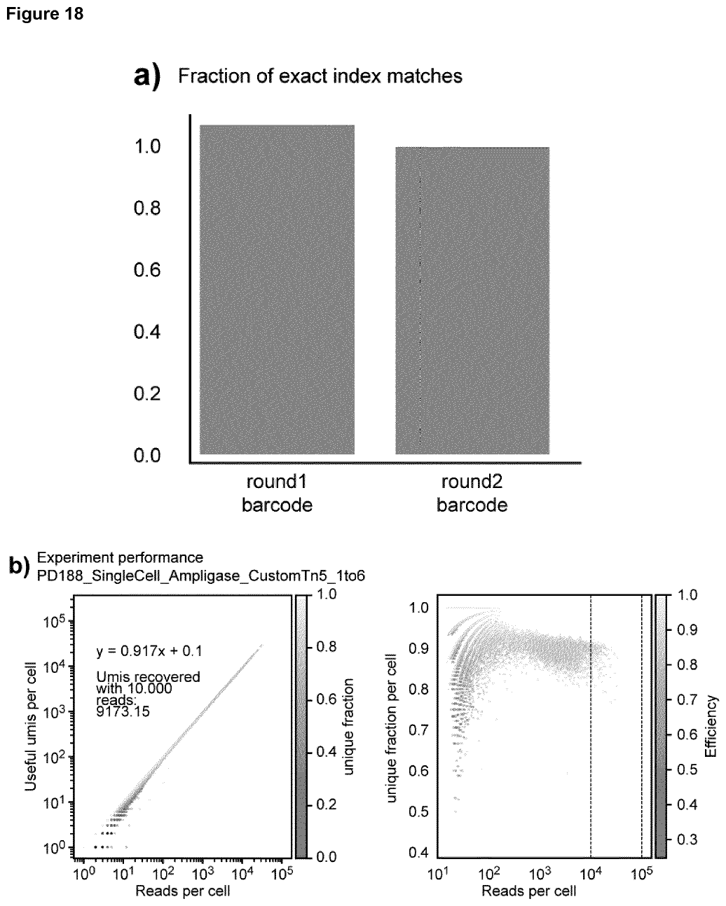

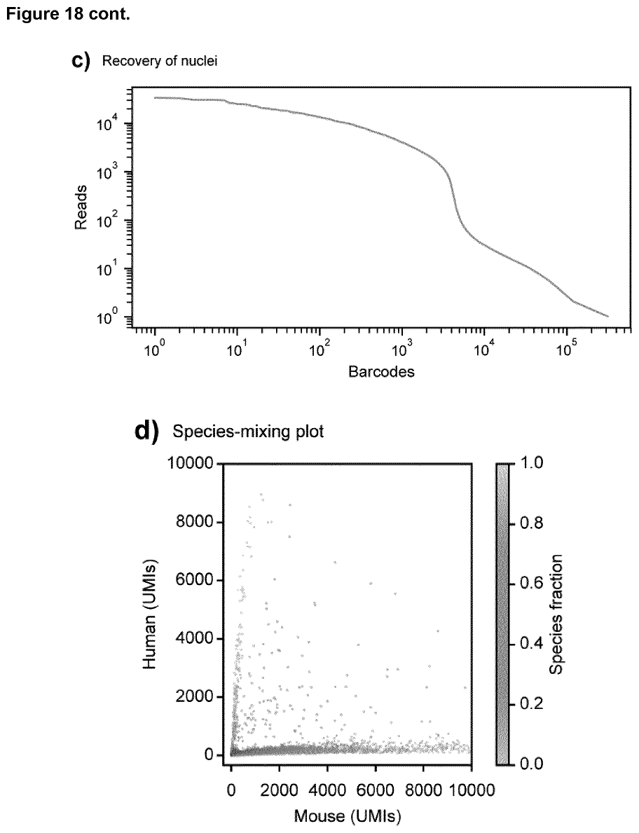

FIG. 18: Sequencing results for scifi-RNA-seq based on thermoligation and template switching

a) Fraction of exact matches for round1 and round2 barcodes. b) Experiment performance of a typical scifi-RNA-seq experiment based on thermoligation and template switching. Left: Reads per cell plotted against unique UMIs per cell reveal that single-cell transcriptomes are highly complex. Right: The rate of unique reads per cell averages around 90% over a wide range of reads sequenced. c) Ranked barcodes plotted against reads reveal a characteristic inflection point that separates cells from background noise. In this particular experiment 15,300 nuclei were loaded into the microfluidic device. d) Species-mixing plot for a 1:1 mixture of human (Jurkat-Cas9-TCR) and mouse (3T3) nuclei.

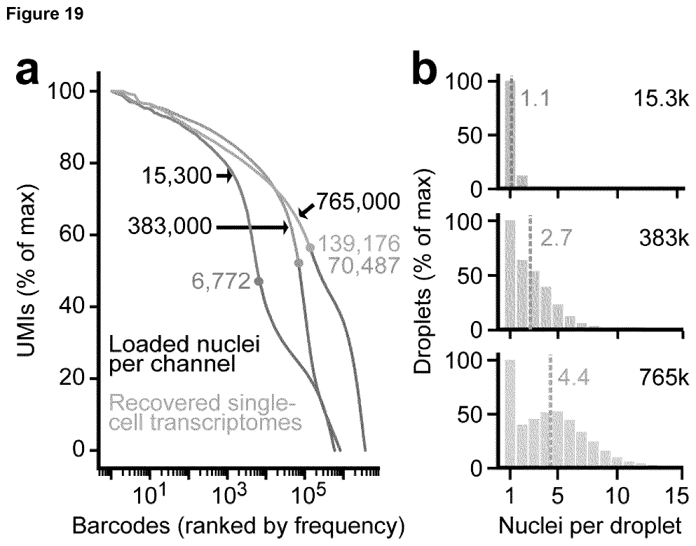

FIG. 19: a) A 1:1 mixture of human and mouse nuclei (Jurkat and 3T3, respectively) was processed with scifi-RNA-seq, loading 15,300, 383,000, and 765,000 nuclei into single microfluidic channels of the Chromium device. Plotting all detected barcodes ranked by frequency against the number of unique molecular identifiers (UMIs) per barcode identifies a characteristic inflection point that separates nuclei from background noise. b) Distribution of the number of nuclei (round1 indices) per droplet (round2 barcode) for increasing nuclei loading concentrations. The average number of nuclei per droplet and nuclei loading concentration per channel are indicated.

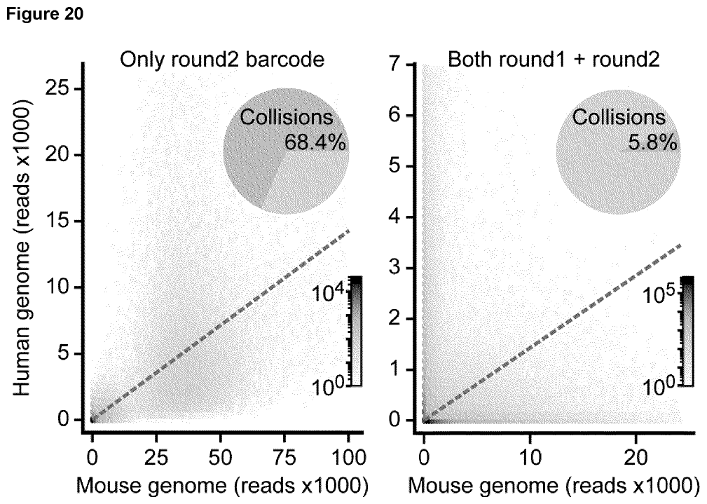

FIG. 20: The round1 transcriptome index can deconvolute multiple nuclei per droplet into the respective single-cell transcriptomes. 765,000 pre-indexed nuclei from a mixture of human (Jurkat) and mouse (3T3) cells were processed in a single microfluidic channel and demultiplexed based on the microfluidic round2 barcode only (left plot), or based on the combination of round1 and round2 barcodes (right plot). The percentages of detected inter-species collisions are shown by the pie charts.

FIG. 21: UMIs per cell and fraction of unique reads per cell were plotted against the number of nuclei contained in the respective droplet, showing no deterioration in the single-cell transcriptome complexity when many cells co-occupy the same droplet. This analysis is based on the largest human/mouse mixing experiment with 765,000 nuclei per microfluidic channel.

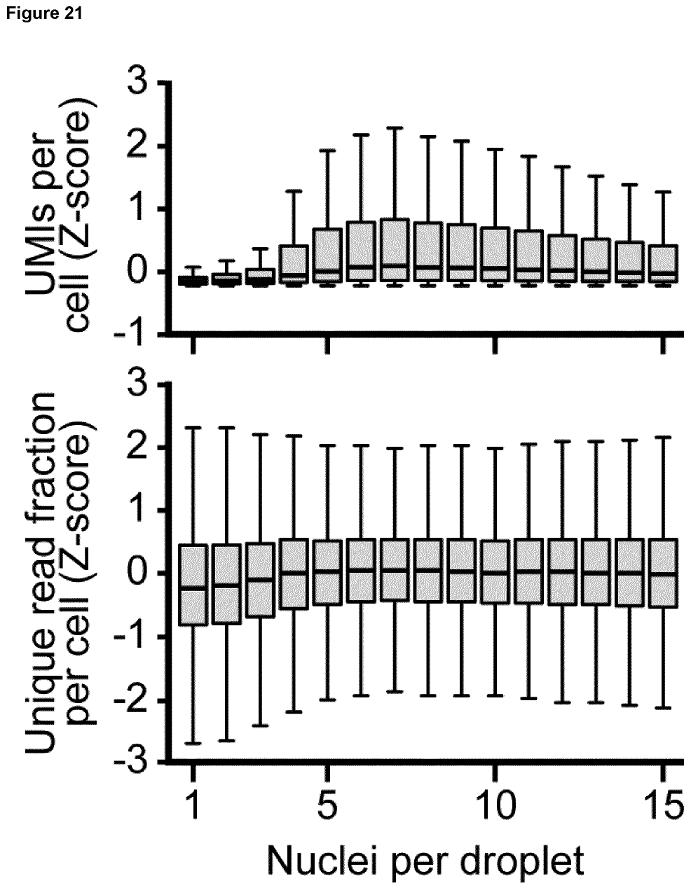

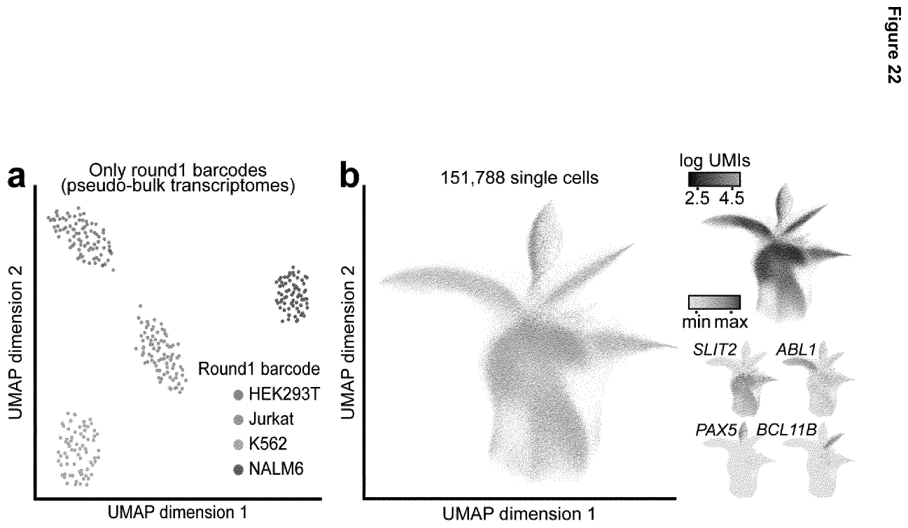

FIG. 22: a) Four human cell lines (HEK293T, Jurkat, K562, NALM6) were processed with scifi-RNA-seq, using defined sets of round1 barcodes for each cell line. Considering only round1 barcodes, the dataset gives rise to averaged pseudo-bulk RNA-seq profiles of the cell lines, which are plotted here. b) 151,788 single-cell transcriptomes derived from the human cell line mixture are displayed in a 2D projection using the UMAP algorithm and colored by round1 barcodes corresponding to cell lines (left), UMIs per cell (top right), or marker gene expression (bottom right).

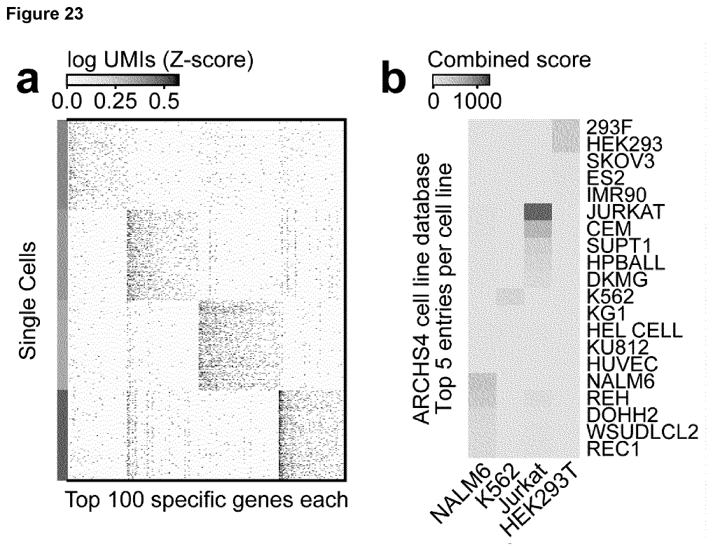

FIG. 23: a) Heatmap showing single-cell expression levels for the top 100 most specific genes for each cell line. We randomly sampled an equal number of single-cell transcriptomes per cell line without filtering for transcriptome quality. b) Gene set enrichment analysis of differentially expressed genes clearly identifies the cell lines.

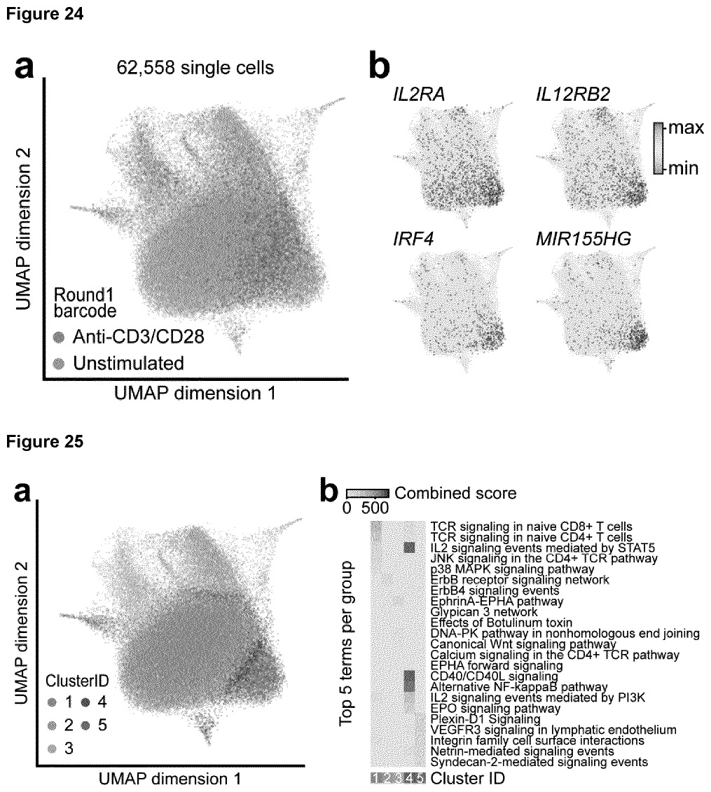

FIG. 24: a) Human primary T cells with or without T cell receptor stimulation were processed using scifi-RNA-seq, and the single-cell transcriptomes are displayed in a UMAP projection (color-coded by stimulation state). b) Expression levels of four genes induced by TCR stimulation overlaid on the UMAP projection.

FIG. 25: a) UMAP projection with single cells colored by clusters assigned by graph-based clustering using the Leiden algorithm. b) Gene set enrichment analysis for the differentially expressed genes in each cluster according to panel k.

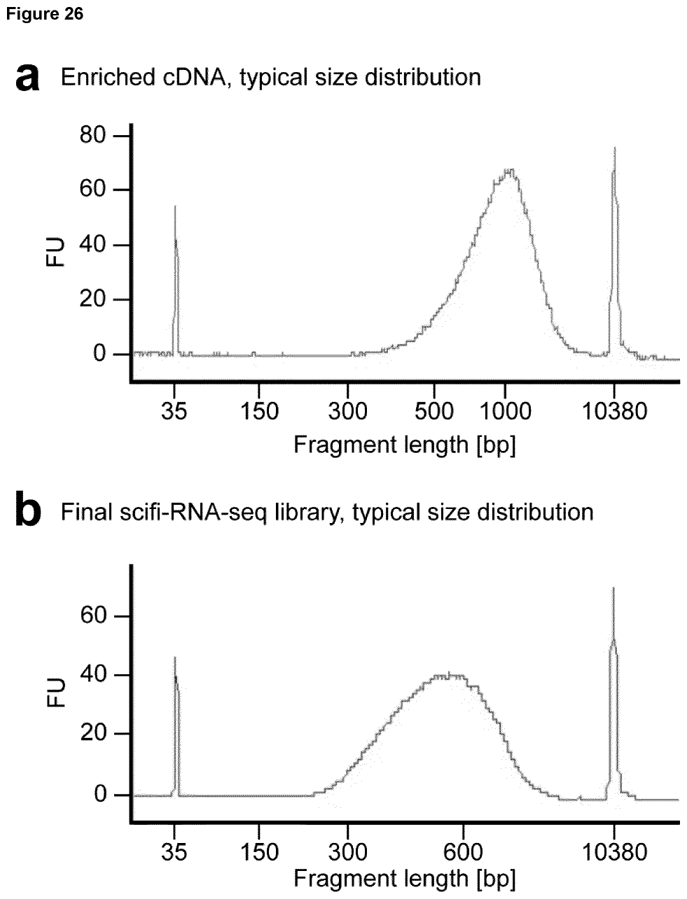

FIG. 26: a) Typical size distribution of enriched cDNA obtained using scifi-RNA-seq. b) Typical size distribution of a final scifi-RNA-seq library ready for next-generation sequencing.

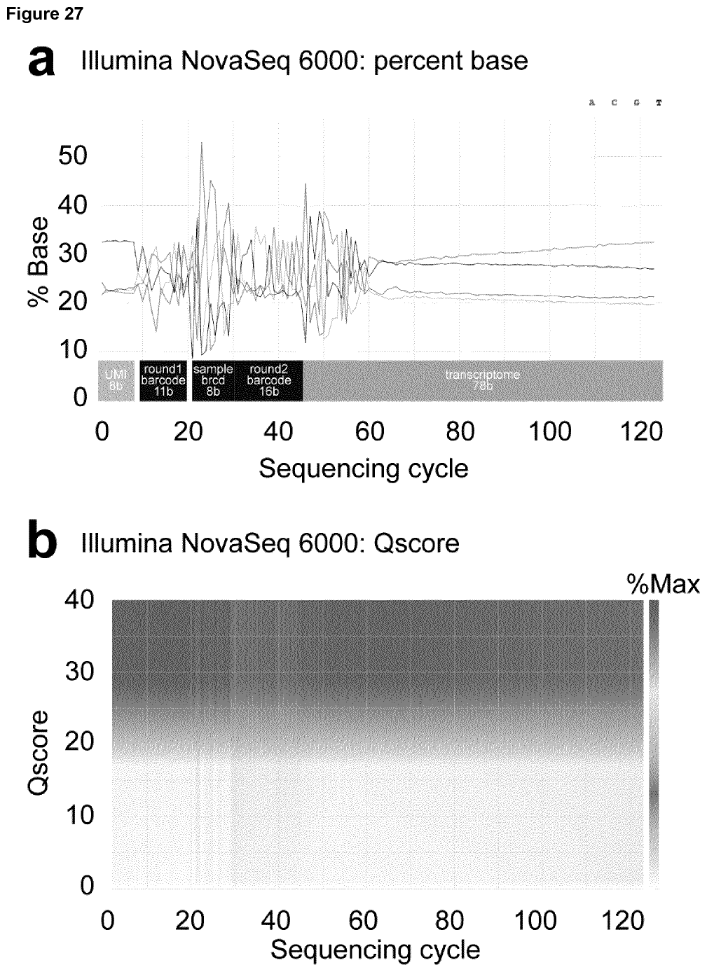

FIG. 27: a) Distribution of DNA bases along scifi-RNA-seq sequencing reads, showing the characteristic sequence patterns of the UMI, round1 barcode, round2 barcode, sample barcode, and transcript. b) Heatmap showing sequencing quality (Qscore) for each sequencing cycle.

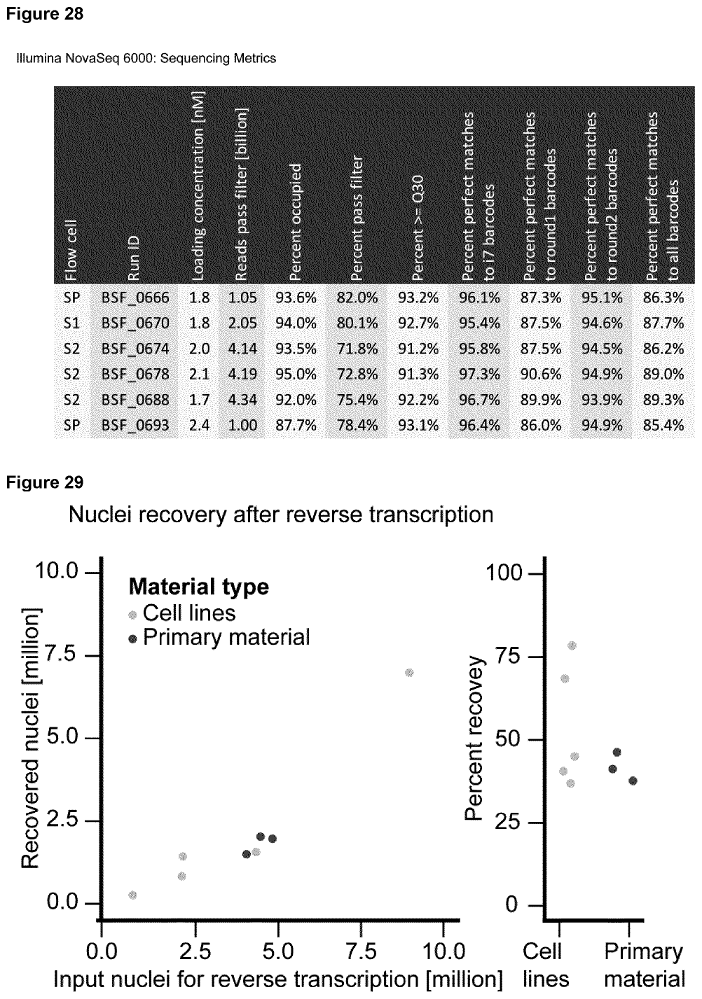

FIG. 28: Table summarizing all NovaSeq 6000 sequencing runs performed as part of this study. scifi-RNA-seq was thoroughly tested with NovaSeq SP, S1, and S2 reagents. The table also summarizes the percentage of reads with perfect match to the sample (i7) barcode, pre-indexing (round1) barcode, microfluidic (round2) barcode, and with a correct combination of all three barcodes.

FIG. 29: Nuclei recovery after pre-indexing of the whole transcriptome by reverse transcription. scifi-RNA-seq achieves high recovery rates for both cell lines and primary material.

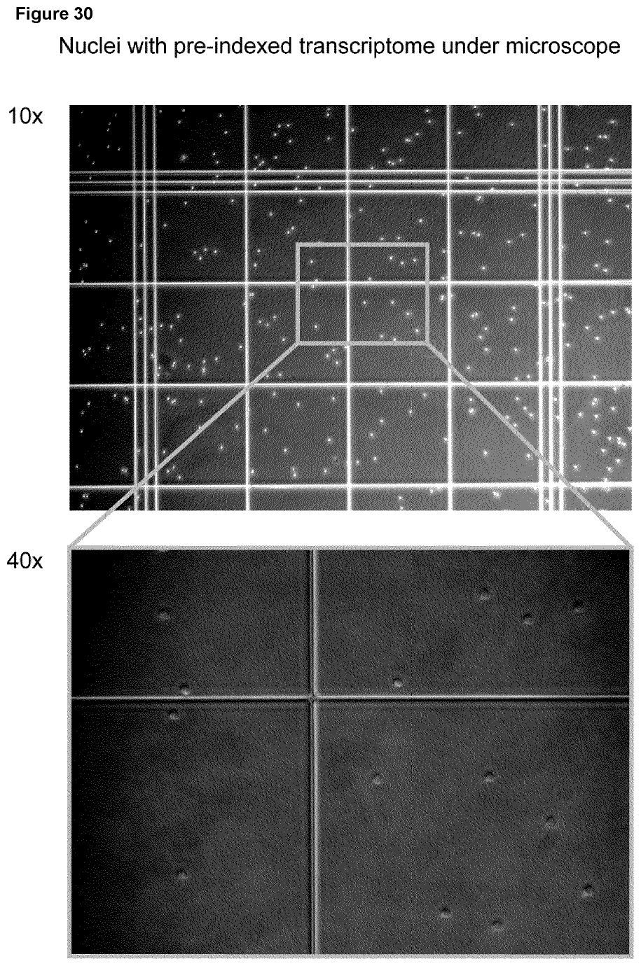

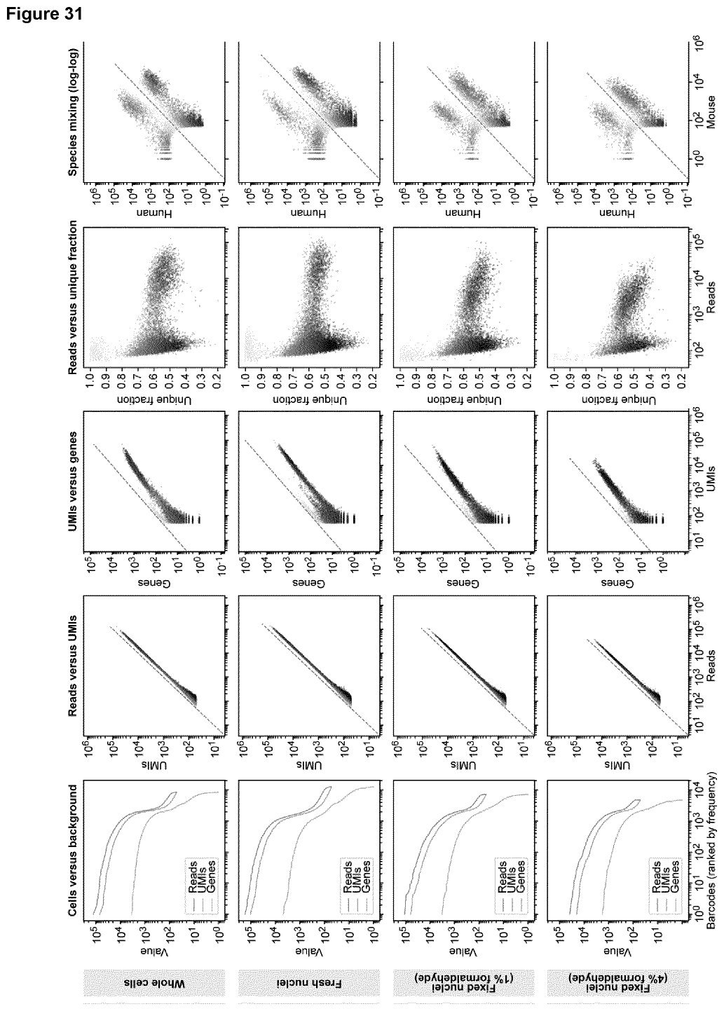

FIG. 30: Nuclei with pre-indexed transcriptome, prior to microfluidic device loading, visualized under a microscope in a counting chamber. The selected images show nuclei derived from human primary T cells.

FIG. 31: A mixture of human (Jurkat) and mouse (3T3) cells was prepared, and scifi-RNA-seq was performed on whole cells permeabilized by methanol, freshly isolated nuclei, and nuclei fixed with 1% or 4% formaldehyde that were cryopreserved, re-hydrated, and permeabilized. During reverse transcription on a 96-well plate, each sample was assigned a specific set of round1 barcodes. Afterward, all wells were pooled, and 15,300 cells/nuclei were loaded into a single channel of the Chromium device. The following performance plots are provided: (i) ranked barcodes plotted against reads, unique molecular identifiers (UMIs), or detected genes, distinguishing single-cell transcriptomes from background noise; (ii) reads plotted against UMIs; (iii) reads plotted against the number of detected genes; (iv) reads plotted against the fraction of unique reads; (v) species mixing plot showing the number of UMIs per cell aligning to the mouse genome (x-axis) versus the human genome (y-axis). To facilitate comparisons between different types of input material, the axes of the performance plots use the same scale across conditions.

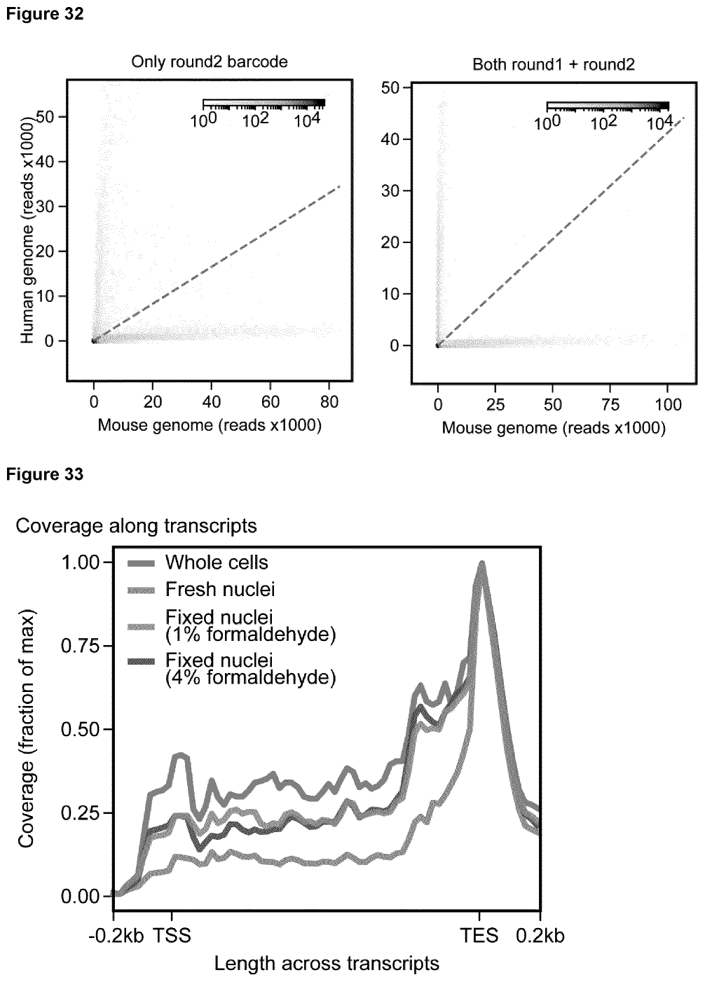

FIG. 32: 15,300 pre-indexed nuclei from a mixture of human (Jurkat) and mouse (3T3) cells were processed in a single microfluidic channel and demultiplexed based on the microfluidic round2 barcode only (left plot), or based on the combination of round1 and round2 barcodes (right plot). At the standard loading concentration of the Chromium device (15,300 nuclei per channel), the microfluidic (round2) index provides sufficient complexity to resolve single cells, although the combination of round1 and round2 barcodes still results in a reduction of background noise.

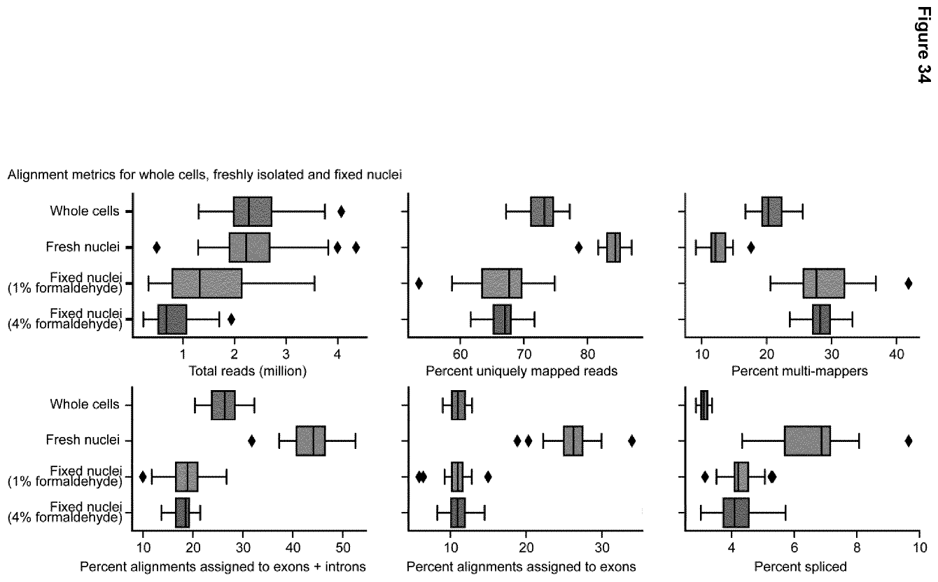

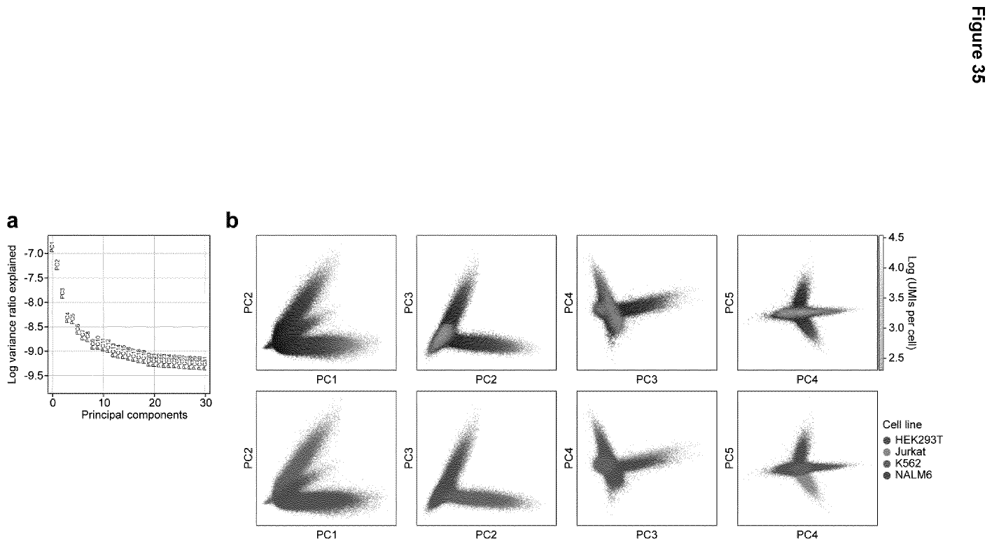

FIG. 33: Coverage along human and mouse transcripts from 200 bp upstream of the transcription start site (TSS) to 200 bp downstream of the transcription end site (TES), shown for whole cells permeabilized by methanol, freshly isolated nuclei, and nuclei fixed with 1% or 4% formaldehyde that were cryopreserved, re-hydrated, and permeabilized. Freshly isolated nuclei show the strongest 3′ enrichment.

FIG. 34: Boxplots summarizing sequence alignment metrics across the different types of input material: Total reads sequenced, percent uniquely mapped reads, percent multi-mappers, percent alignments to exons plus introns, percent alignments to exons, and percent spliced reads. Freshly isolated nuclei showed the best performance for these alignment metrics.

FIG. 35: Principal component analysis for a scifi-RNA-seq experiment on a 1:1:1:1 mixture of four human cell lines with unique characteristics. a) Variance explained by the top 30 principal components. b) Principal component analysis (PCA) projections for 151,788 single cells, color-coded with the number of UMIs per cell (top row) and with round1 barcodes denoting cell lines.

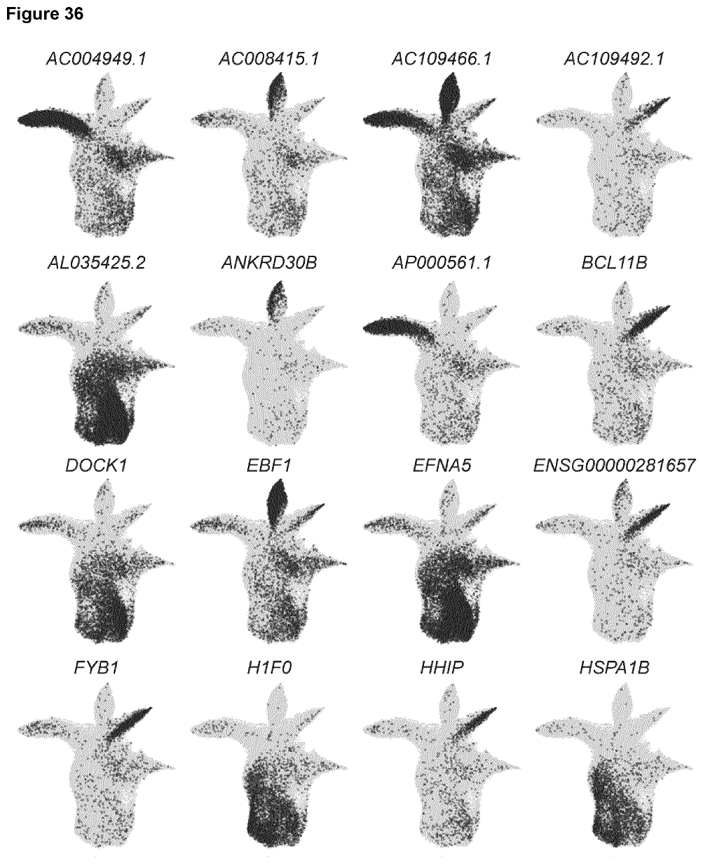

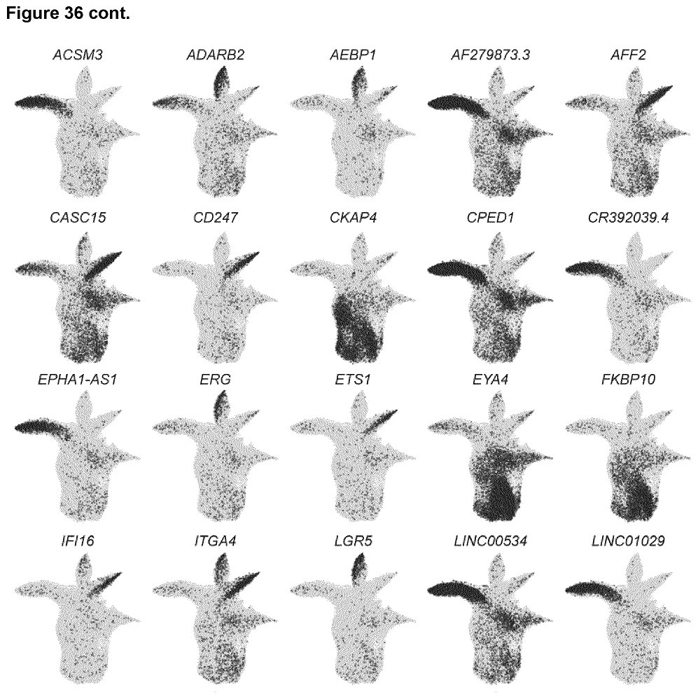

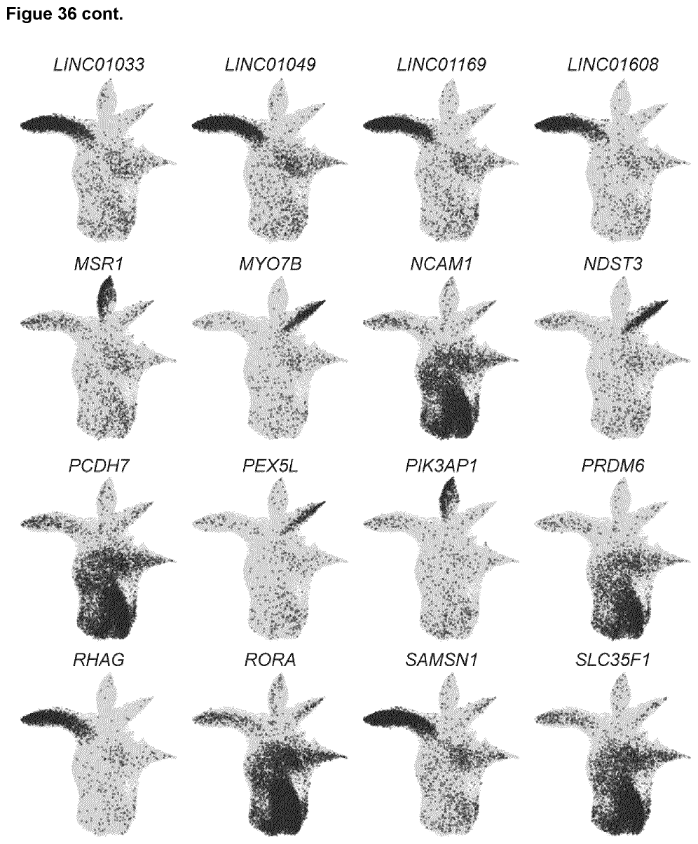

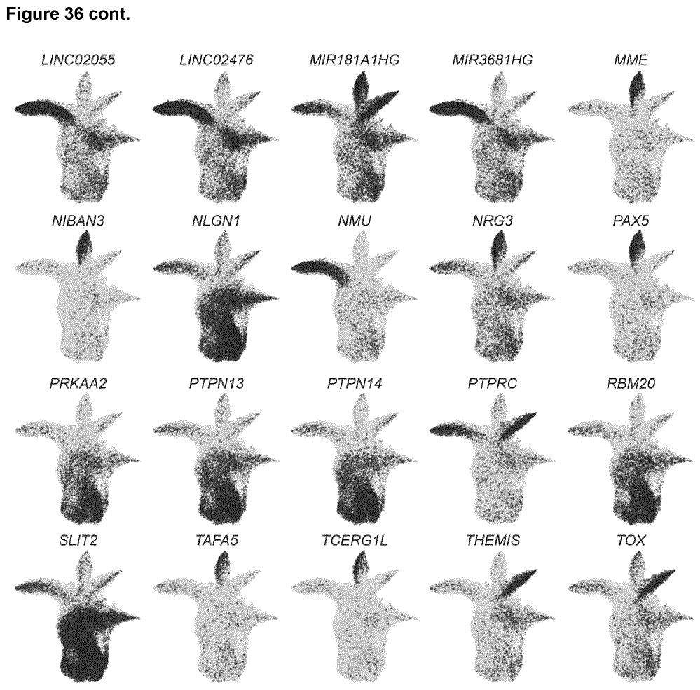

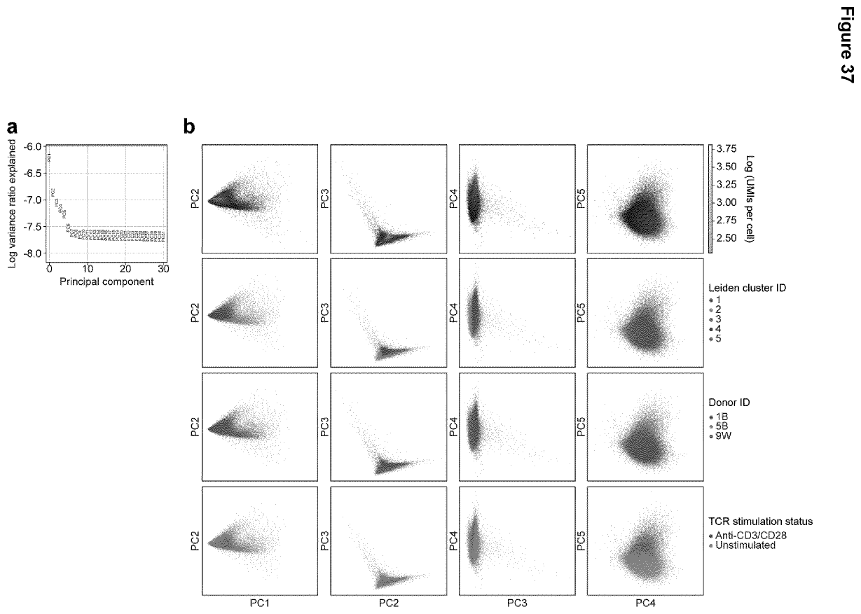

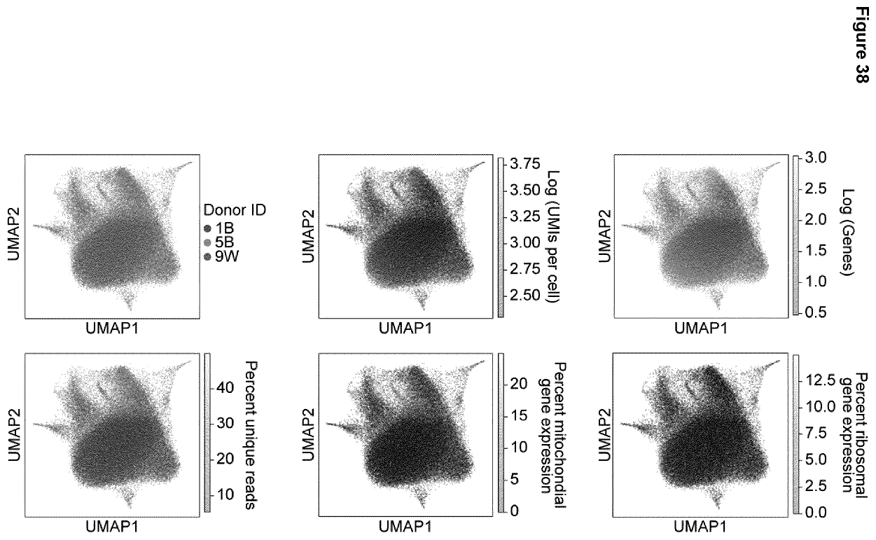

FIG. 36: Expression values of 72 additional cell line specific genes mapped onto the UMAP projection as shown in FIG. 22.

FIG. 37: Principal component analysis for a scifi-RNA-seq experiment on primary human T cells with or without T cell receptor stimulation. a) Variance explained by the top 30 principal components. b) PCA projections for 62,558 single cells. From top to bottom, the following variables are mapped onto these projections: Logarithm of UMIs per cell, cluster ID, donor ID, and T cell receptor (TCR) stimulation status.

FIG. 38: UMAP projections for 62,558 single cells (as shown in FIG. 24) with additional variables mapped onto the projections: Donor ID, logarithm of UMIs per cell, logarithm of detected genes per cell, percent unique reads per cell, percent mitochondrial expression, and percent ribosomal expression.

FIG. 39: a) An equal mixture of four human cell lines (HEK293T, Jurkat, K562, NALM6) was processed in parallel with scifi-RNA-seq and 10× Genomics v3 profiling, using intact cells, nuclei or methanol-fixed cells as input. To allow a direct comparison between the platforms, we loaded a standardized concentration of 7,500 cells/nuclei per microfluidic channel. To assess cell/nuclei recovery rates, we plotted all detected barcodes ranked by frequency against the number of unique molecular identifiers (UMIs) per barcode. b) Dimensionality reduction (UMAP) and clustering with the Leiden algorithm readily identify the four cell lines in all samples. For the Chromium system we detected additional, spurious clusters that are mixtures of the cell lines (gray), which are completely absent from scifi-RNA-seq data. c) Despite their highly distinct transcript content, cell lines are recovered at equal proportions. d) Clustering of gene expression profiles based on Pearson correlation grouped samples by cell line, irrespective of the technology or cell preparation method used.

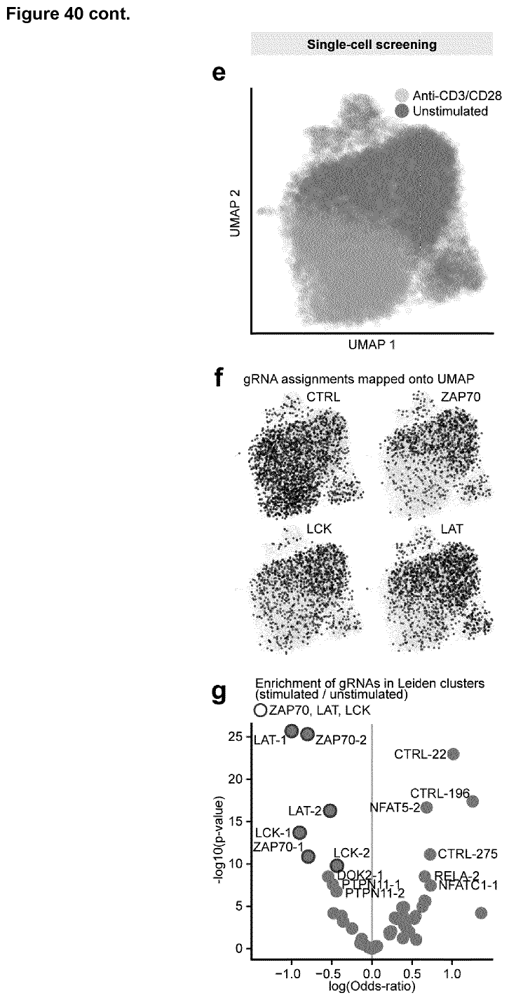

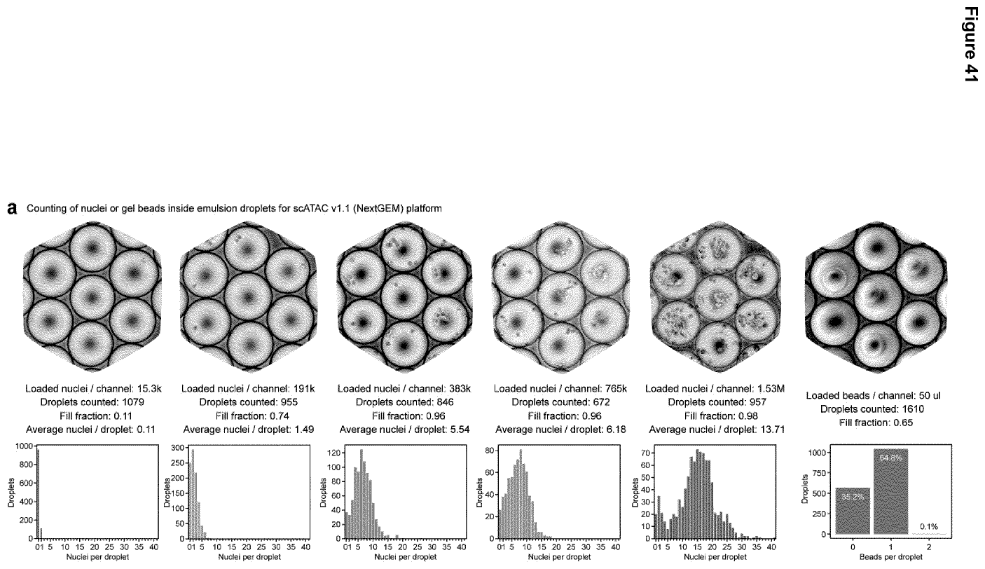

FIG. 40: a) Human Jurkat cells expressing Cas9 were transduced in an arrayed format with lentiviral constructs encoding 48 distinct gRNAs. After efficient genome editing, samples were split and stimulated with anti-CD3/CD28 beads to activate the T cell receptor (TCR) or were left untreated. The plate was processed with scifi-RNA-seq, labeling CRISPR perturbations and the treatment with specific round1 reverse transcription barcodes. This proof-of-concept screen demonstrates the potential of scifi round1 multiplexing for genetic perturbation and drug screens with hundreds to thousands of conditions, which is useful for drug development. b) Principal component analysis of 96 bulk transcriptomes colored by the treatment and labeled with the genetic perturbation. Key activators of the TCR pathway are highlighted with a circle. c) The top 300 differentially expressed genes between stimulated and unstimulated control cells were used as a screening signature. A heatmap for this gene set was prepared (data not shown). Gene perturbations were assigned a TCR activation score based on the expression of these genes. Samples have been sorted by TCR activation score. Some gene knockouts result in a decreased TCR activation score similar to unstimulated samples. d) TCR activation score based on the transcriptome plotted against a proliferation score derived from cell counts. e) Single-cell transcriptomes derived from the CRISPR screen are displayed in a 2D projection using the UMAP algorithm and colored by the TCR treatment. f) Cells assigned to control gRNAs, or gRNAs targeting ZAP70, LCK, LAT are highlighted in black. g) Enrichment of gRNAs in the Leiden cluster identified as stimulated over the unstimulated cluster. gRNAs targeting ZAP70, LAT, LCK are highlighted with a circle.

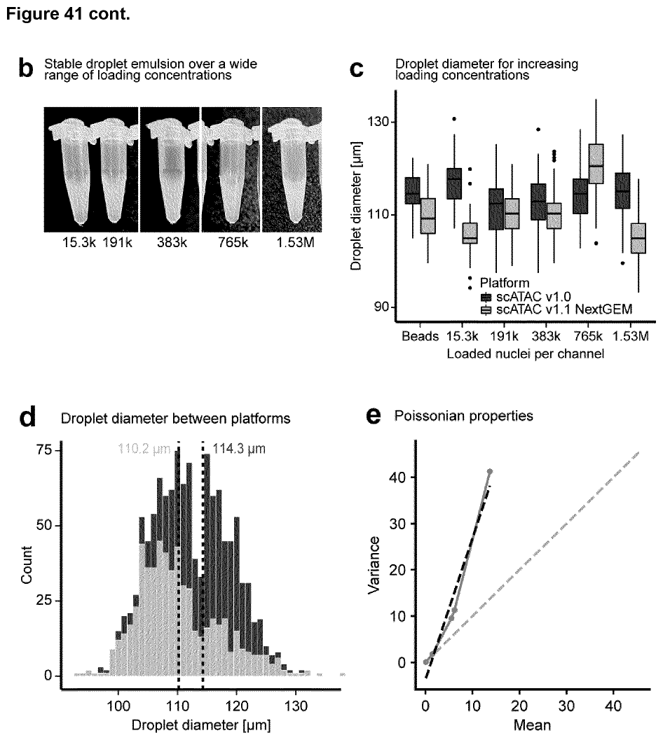

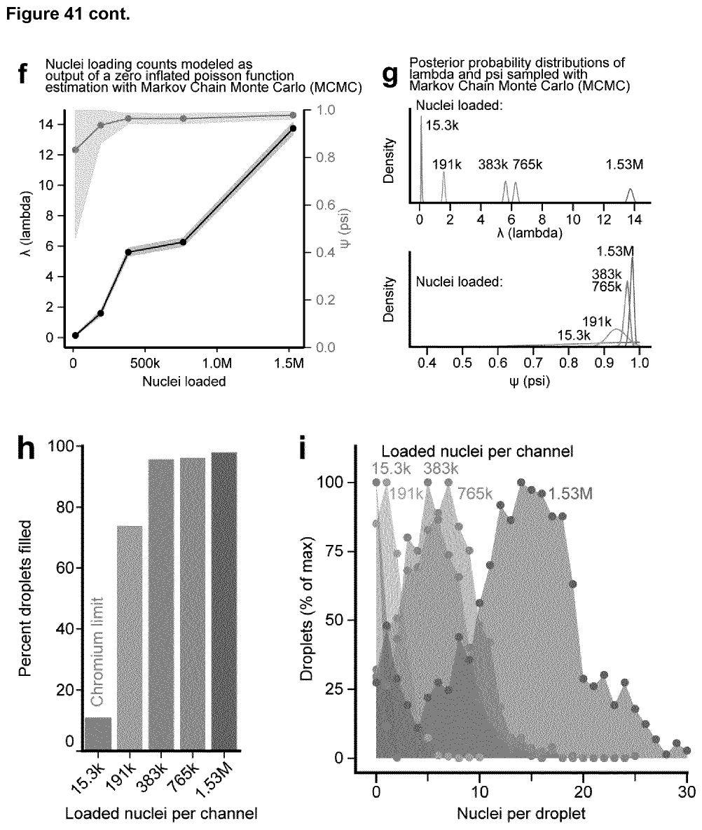

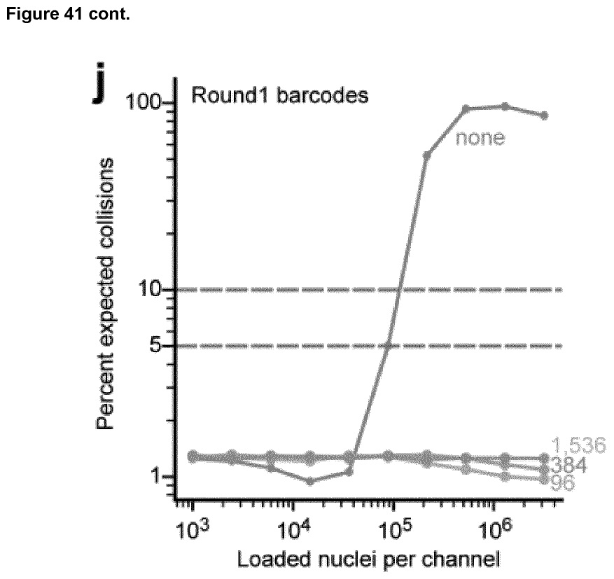

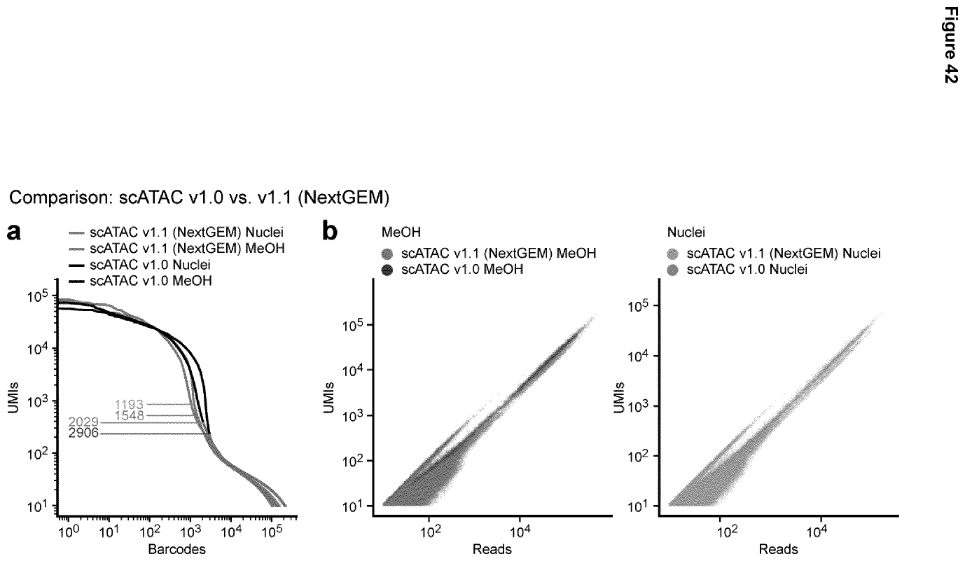

FIG. 41: a) Droplet overloading experiments repeated on the Chromium NextGEM platform. By omitting the lysis reagents, nuclei remained intact and were imaged using a standard microscope, allowing the counting of nuclei per droplet. Results for loading concentrations of 15,300, 191,000, 383,000, 765,000, and 1,530,000 nuclei per channel are summarized as histograms. For each loading concentration, the number of evaluated droplet images, the droplet fill fraction, and the average number of nuclei per droplet are shown. Furthermore, by substituting the nuclei suspension with 1× Nuclei Buffer and omitting Reducing Agent B, intact gel beads were visualized inside the emulsion droplets. Bead fill rates based on 1,610 evaluated droplet images are shown. b) Despite substantial droplet overloading, we obtained stable droplet emulsions for all tested conditions. c) Droplet diameter compared between the scATAC 1.0 and scATAC 1.1 (NextGEM platforms), for increasing loading concentrations. Per condition, 100 droplets were measured. d) Droplet diameter displayed as histogram. Data for different loading concentrations was pooled, for a total of 500 droplets per platform. e) Nuclei loading displays properties of a Poisson-like distribution. The mean is plotted on the x-axis against the variance on the y-axis. f) Computational modeling of nuclei loading as a zero-inflated Poisson function. g) Posterior probability distributions of lambda and psi sampled with Markov Chain Monte Carlo (MCMC). h) Droplet overloading boosts the percentage of droplets filled with nuclei for the NextGEM platform. i) Droplet overloading causes the average number of nuclei per droplet to increase in a controlled fashion while maintaining the desired Poisson-like loading distribution. j) Expected collision rate as a function of the cell/nuclei loading concentration per channel for standard Chromium profiling and for defined sets of round1 barcodes. The cell/nuclei fill rate was modelled as a zero-inflated Poisson distribution.

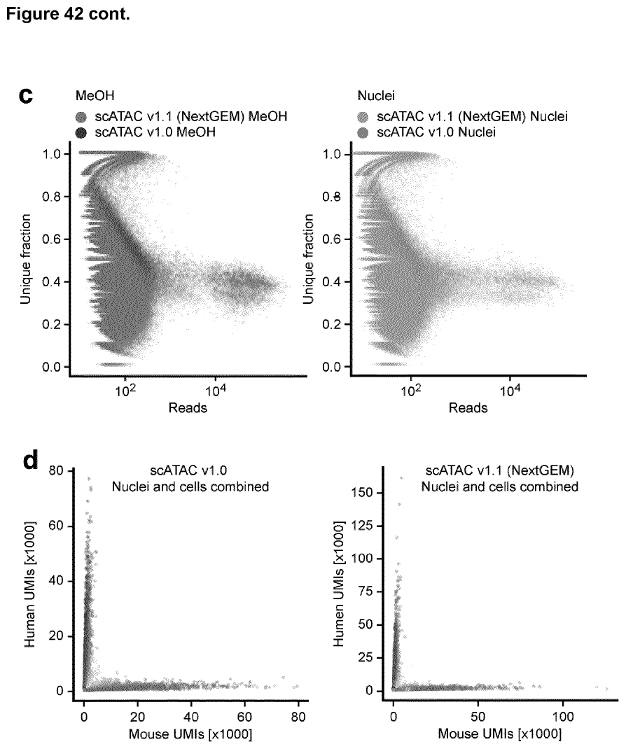

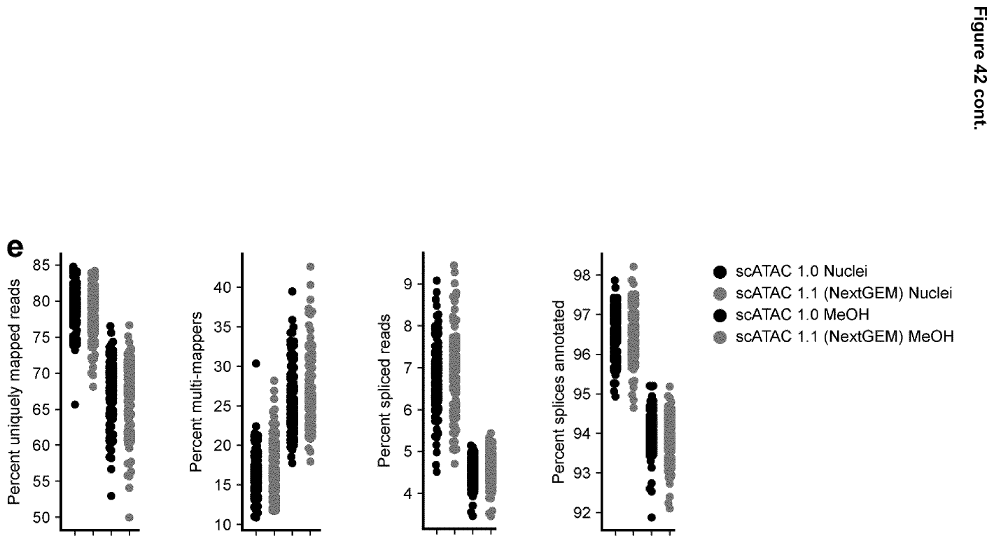

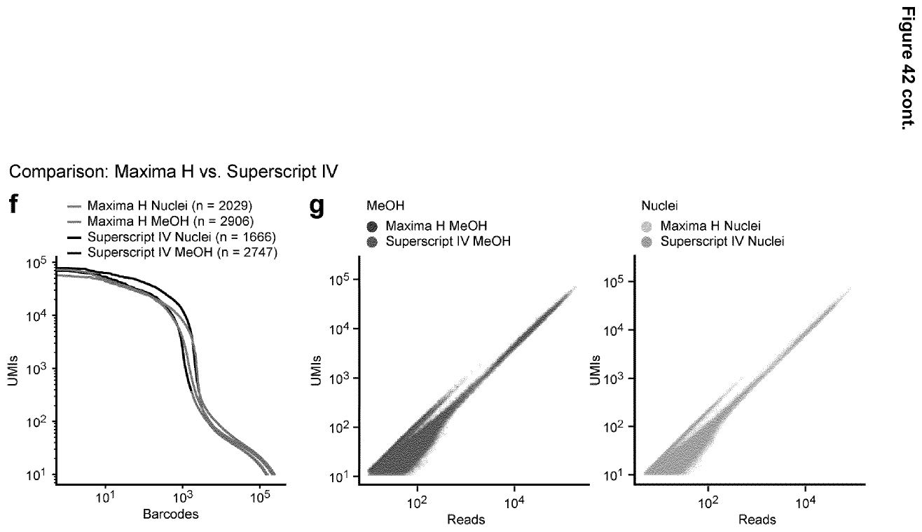

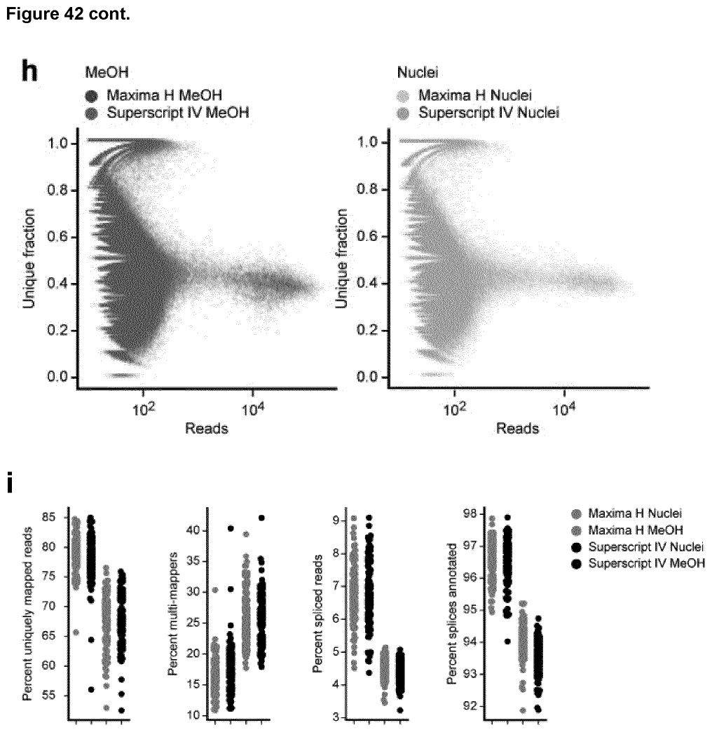

FIG. 42: a) Cell barcodes ranked by frequency versus UMIs per cell. b) Reads per cell plotted against UMIs per cell to assess the level of sequencing saturation. c) Reads per cell plotted against the unique read fraction per cell so assess PCR duplication and library complexity. d) Alignments to the human genome versus alignments to the mouse genome. e) Alignment metrics compared between the ScATAC 1.0 and 1.1 (NextGEM) platforms. f) Cell barcodes ranked by frequency versus UMIs per cell. g) Reads per cell plotted against UMIs per cell to assess the level of sequencing saturation. h) Reads per cell plotted against the unique read fraction per cell so assess PCR duplication and library complexity. i) Alignment metrics for scifi-RNA-seq using Maxima H Minus compared to Superscript IV reverse transcriptase for the reverse transcription step. The template switching was performed with Maxima H Minus reverse transcriptase in both cases.

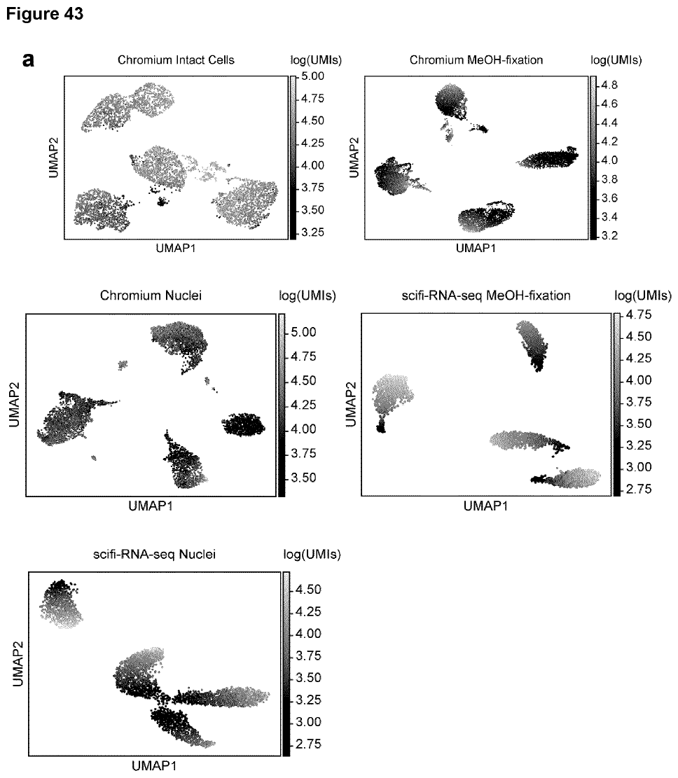

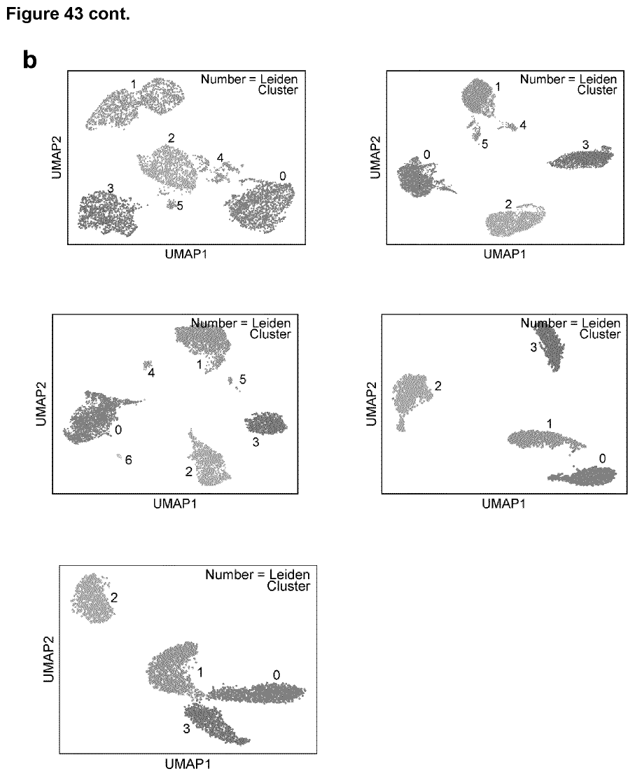

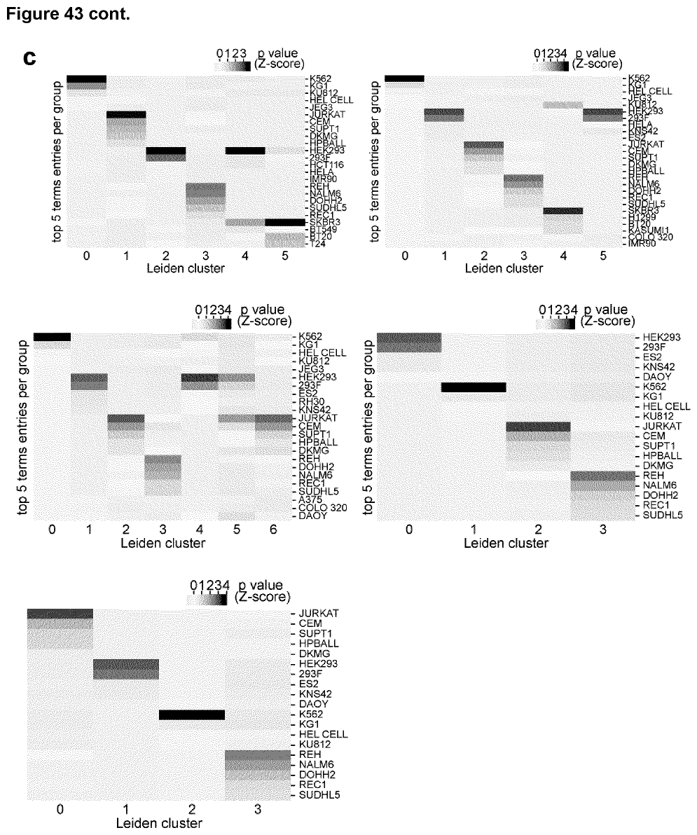

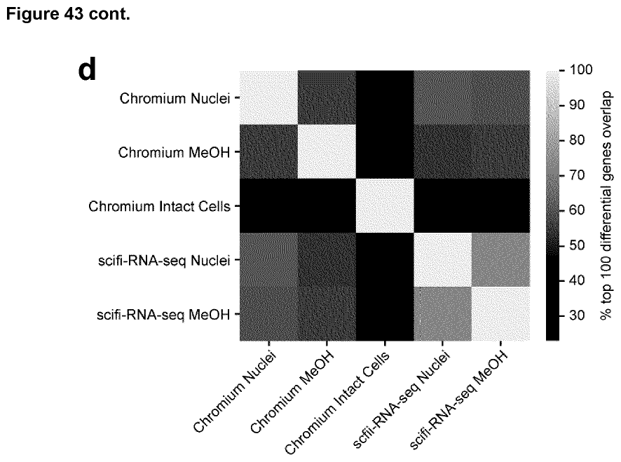

FIG. 43: An equal mixture of four human cell lines (HEK293T, Jurkat, K562, NALM-6) was processed in parallel with scifi-RNA-seq and the Chromium v3 Single Cell Gene Expression kit. a) Single-cell transcriptomes are displayed in a 2D projection using the UMAP algorithm with the number of UMIs per cell mapped on top. b) Clustering of single cells with the Leiden algorithm, with cluster IDs mapped onto the UMAP projection. c) Enrichment of cell line signatures obtained from the ARCHS4 database for the identified Leiden clusters. These results can be used to label clusters with their respective cell line, and to identify spurious clusters of doublet cells. d) Percent overlap of the top 100 differentially expressed genes between samples.

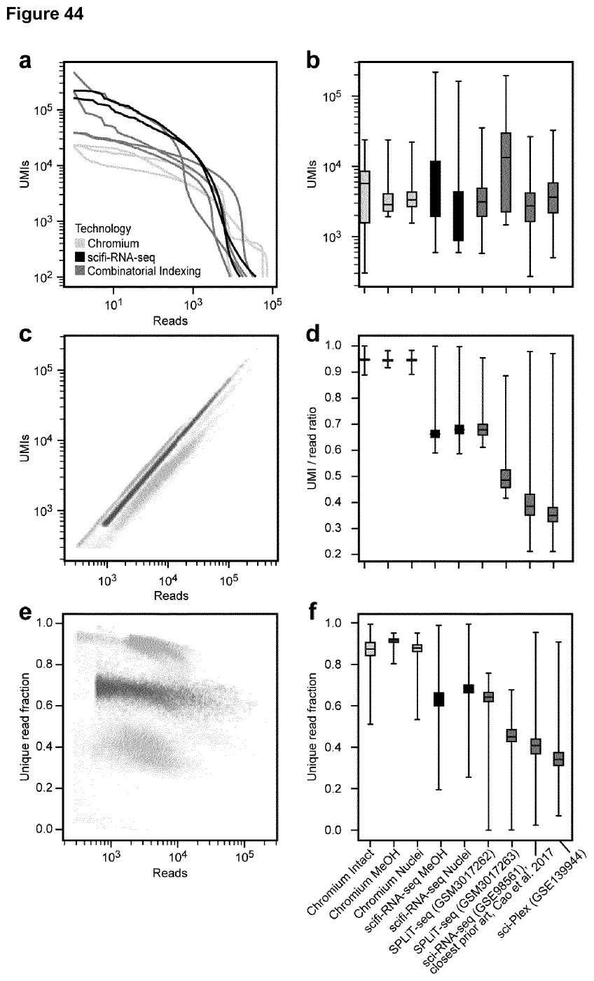

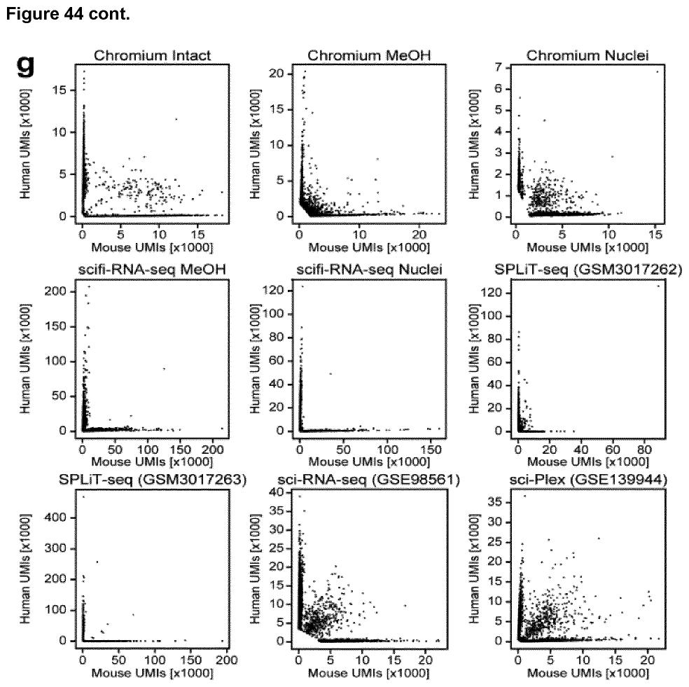

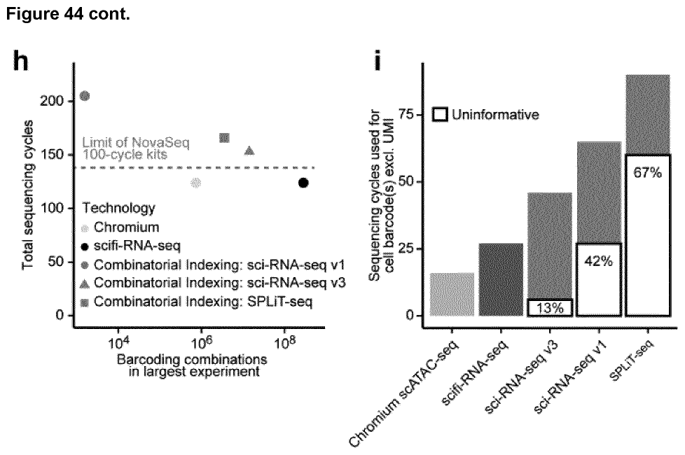

FIG. 44: Technology comparison between scifi-RNA-seq and existing, multi-round combinatorial indexing approaches or the 10× Genomics Chromium platform. For this comparison publicly available combinatorial indexing data was obtained, including that of Cao et al., 2017. The Cao et al., 2017 dataset is highlighted in the Figure. A species mixture of human Jurkat cells and mouse 3T3 cells was also processed in parallel with the methods of the invention and the 10× Genomics Chromium workflow. a) Detected cell barcodes ranked by frequency plotted against the number of unique molecular identifiers (UMIs) per barcode. b) UMI counts summarized as a bar plot. c) Reads per cell plotted against UMIs per cell, to assess sequencing saturation. d) UMIs over read ratio, as a metric for PCR duplication. e) Reads per cell plotted against the fraction of unique reads per cell. f) Unique read fraction summarized as a bar plot. g) Alignments to the human genome versus alignments to the mouse genome. h) Barcoding combinations in the largest, actually performed experiment against the total number of sequencing cycles used in that experiment. The grey line shows the 138 sequencing cycles included in the NovaSeq 100-cycle kits. i) Sequencing cycles used for reading the composite cell barcode (excluding the UMI). Uninformative sequencing cycles from ligation overhangs, primer binding sites and transposase mosaic ends are depicted in gray, and the percentage of uninformative sequencing cycles is provided. In summary, it could be shown consistently that superior data quality over the method of Cao et al., 2017 and over all other published combinatorial indexing methods can be achieved with the methods of the invention. scifi-RNA-seq also provides an at least 15-fold increased cell throughput compared to 10× Genomics Chromium.

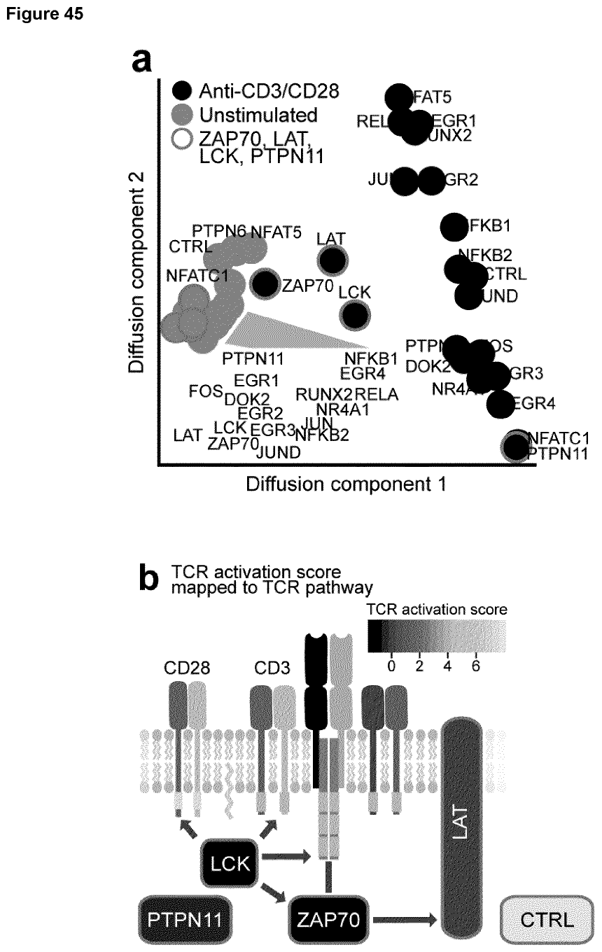

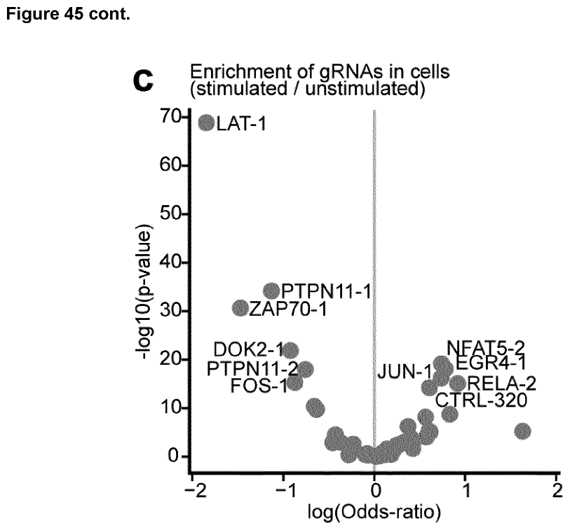

FIG. 45: a) Diffusion map of 96 bulk transcriptomes (48 CRISPR knockouts, 2 treatments), colored by the treatment and labeled with the gene perturbation. Key regulators of the T cell receptor (TCR) pathway are highlighted with circles. Knockout of ZAP70, LAT and LCK makes cells more similar to unstimulated samples. b) TCR activation signature defined in FIG. 3c, mapped onto a schematic of TCR pathway activation. c) Enrichment of cells with the indicated gRNAs in the stimulated over the unstimulated group. This is a measurement of proliferation, in contrast to the TCR activation that we define based on the transcriptome.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While the invention has been illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention also covers all further features shown in the figures individually, although they may not have been described in the afore or following description. Also, single alternatives of the embodiments described in the figures and the description and single alternatives of features thereof can be disclaimed from the subject matter of the other aspect of the invention.

Furthermore, in the claims the word “comprising” does not exclude other elements or steps, and the indefinite article “a” or “an” does not exclude a plurality. A single unit may fulfil the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

### Example 1-Preparation of Cells/Nuclei

1.1 Preparation of Permeabilized Whole Cells from Human and Mouse Cell Lines

5 million cells were washed with 10 ml of ice-cold 1×PBS (Gibco cat. no. 14190-094, centrifugation: 300 rcf, 5 min, 4° C.) and fixed in 5 ml of ice-cold methanol (Fisher Scientific cat. no. M/4000/17) at −20° C. for 10 min. After two additional washes (centrifugation: 300 rcf, 5 min, 4° C.) with 5 ml of ice-cold PBS-BSA-SUPERase (1×PBS supplemented with 1% w/v BSA (Sigma cat. no. A8806-5) and 1% v/v SUPERase-In RNase Inhibitor (Thermo Fisher Scientific cat. no. AM2696)) permeabilized cells were resuspended in 200 μl of ice-cold PBS-BSA-SUPERase, and filtered through a cell strainer (40 UM or 70 UM depending on the cell size). 10 μl of the sample were used for cell counting on a CASY device (Schärfe System), and diluted to 5,000 cells per μl with ice-cold PBS-BSA-SUPERase. It was immediately proceeded with the reverse transcription step.

1.2 Preparation of Fresh Nuclei from Human and Mouse Cell Lines

5 million cells were washed with 10 ml of ice-cold 1×PBS (Gibco cat. no. 14190-094, 300 rcf, 5 min, 4° C.). Nuclei were prepared by resuspending cells in 500 μl of ice-cold Nuclei Preparation Buffer (10 mM Tris-HCl PH 7.5 (Sigma cat. no. T2944-100ML), 10 mM NaCl (Sigma cat. no. S5150-1L), 3 mM MgCl2 (Ambion cat. no. AM9530G), 1% w/v BSA (Sigma cat. no. A8806-5), 1% v/v SUPERase-In RNase Inhibitor (Thermo Fisher Scientific cat. no. AM2696), 0.1% v/v Tween-20 (Sigma cat. no. P7949-500ML), 0.1% v/v IGEPAL CA-630 (Sigma cat. no. 18896-50ML), 0.01% v/v Digitonin (Promega cat. no. G944A)), followed by 5 min of incubation on ice. Lysis of the plasma membrane was stopped by adding 5 ml of ice-cold Nuclei Wash Buffer (10 mM Tris-HCl PH 7.5, 10 mM NaCl, 3 mM MgCl2, 1% w/v BSA, 1% v/v SUPERase-In Rnase Inhibitor, 0.1% v/v Tween-20). Nuclei were collected by centrifugation (500 rcf, 5 min, 4° C.), resuspended in 200 μl of ice-cold PBS-BSA-SUPERase (1×PBS supplemented with 1% w/v BSA and 1% v/v SUPERase-In Rnase Inhibitor (20 U/μl, cat. no.)) and filtered through a cell strainer (40 μM or 70 μM depending on the cell size). 10 μl of the sample were used for cell counting on a CASY device (Schärfe System), and diluted to 5,000 cells per μl with ice-cold PBS-BSA-SUPERase. It was immediately proceeded with the reverse transcription step.

1.3 Preparation of Nuclei from Primary Cells with Formaldehyde Fixation and Permeabilization

5 million primary cells were washed with 10 ml of ice-cold 1×PBS (Gibco cat. no. 14190-094, centrifugation: 300 rcf, 5 min, 4° C.). Nuclei were prepared by resuspending cells in 500 μl of ice-cold Nuclei Preparation Buffer without Digitonin and without Tween-20 (10 mM Tris-HCl PH 7.5 (Sigma cat. no. T2944-100ML), 10 mM NaCl (Sigma cat. no. S5150-1L), 3 mM MgCl2 (Ambion cat. no. AM9530G), 1% w/v BSA (Sigma cat. no. A8806-5), 1% v/v SUPERase-In RNase Inhibitor (Thermo Fisher Scientific cat. no. AM2696), 0.1% v/v IGEPAL CA-630 (Sigma cat. no. 18896-50ML)), followed by 5 min of incubation on ice. Lysis of the plasma membrane was stopped by addition of 5 ml of Nuclei Wash Buffer without Tween-20 (10 mM Tris-HCl pH 7.5, 10 mM NaCl, 3 mM MgCl2, 1% w/v BSA, 1% v/v SUPERase-In Rnase Inhibitor). Nuclei were collected by centrifugation (500 rcf, 5 min, 4° C.), and fixed in 5 ml of ice-cold 1×PBS containing 4% Formaldehyde (Thermo Fisher Scientific cat. no. 28908) for 15 min on ice. Fixed nuclei were collected (500 rcf, 5 min, 4° C.), the pellet was resuspended in 1.5 ml of ice-cold Nuclei Wash Buffer without Tween-20 and transferred to a 1.5 ml tube. After one more wash with 1.5 ml of ice-cold Nuclei Wash Buffer without Tween-20 (500 rcf, 5 min, 4° C.), fixed nuclei were resuspended in 200 μl of Nuclei Wash Buffer without Tween-20, snap-frozen in liquid nitrogen and stored at −80° C.

For processing with scifi-RNA-seq, frozen samples were thawed in a 37° C. water bath for exactly 1 min, and immediately placed on ice. Following centrifugation (500 rcf, 5 min, 4° C.), fixed nuclei were resuspended in 250 μl of ice-cold Permeabilization Buffer (10 mM Tris-HCl, 10 mM NaCl, 3 mM MgCl2, 1% w/v BSA, 1% v/v SUPERase-In Rnase Inhibitor, 0.01% v/v Digitonin (Promega cat. no. G944A), 0.1% v/v Tween-20 (Sigma cat. no P7949-500ML)). After 5 min of incubation in ice, 250 μl of Nuclei Wash Buffer without Tween-20 were added per sample, and nuclei were collected (500 rcf, 5 min, 4° C.). After one more wash with 250 μl of Nuclei Wash Buffer without Tween-20, nuclei were taken up in 100 μl of 1×PBS containing 1% w/v BSA and 1% v/v SUPERase-In Rnase Inhibitor. 5 μl of the sample were used for cell counting on a CASY device (Schärfe Systems), and diluted to 5,000 cells per μl with PBS-BSA-SUPERase. It was immediately proceeded with the reverse transcription step.

### Example 2-Testing of Equipment

### 2.1 Testing the Nuclei Loading Capacity of the Chromium Controller

Human Jurkat cells (clone E6-1) were cultured in RPMI medium (Gibco cat. no. 21875-034) supplemented with 10% FCS (Sigma) and penicillin-streptomycin (Gibco cat. no. 15140122). Fresh nuclei were isolated as described above. Next, samples of 15.3 k, 191 k, 383 k, 765 k and 1.53M nuclei were prepared, 1.5 μl of Reducing Agent B (10× Genomics cat. no. 2000087) and 1× Nuclei Buffer (10× Genomics cat. no. 2000153) were added to a total volume of 80 μl. This buffer does not contain detergents, hence the nuclei remain intact during the microfluidic run and can be visualized inside the emulsion droplets with a standard light microscope. At the same time, Reducing Agent B dissolves the Gel Beads, which might otherwise obstruct the view. The microfluidic chip (Single Cell E Chip, 10× Genomics 2000121) was loaded as follows: 75 μl of nuclei sample at the indicated loading concentrations into inlet 1, 40 μl of Single Cell ATAC Gel Beads (10× Genomics cat. no. 2000132) into inlet 2, and 240 μl of Partitioning Oil (10× Genomics cat. no. 220088) into inlet 3. To image the resulting droplets, 15 μl of Partitioning Oil were pipetted onto a glass slide, followed by 5 μl of emulsion droplets, and images were taken at 10× magnification. An average of 653 droplets per condition were counted.

### 2.2 Measuring the Bead Fill Rate of the Chromium Controller

To measure the bead fill rate, the Single Cell E Chip (10× Genomics 2000121) was loaded with 80 μl of 1× Nuclei Buffer (10× Genomics cat. no. 2000153) into inlet 1, 40 μl of Single Cell ATAC Gel Beads (10× Genomics cat. no. 2000132) into inlet 2, and 240 μl of Partitioning Oil (10× Genomics cat. no. 220088) into inlet 3. By leaving out Reducing Agent B, it was ensured that Gel Beads remain intact throughout the microfluidic run, such that they can be visualized inside the emulsion droplets using a standard light microscope. The fill rate calculations are based on a total of 1,265 droplets.

### Example 3-Scifi-RNA-Seq Based on Linear Extension and a Custom Tn5 Transposome (Version EXT-TN5)

Reverse Transcription: Sets of 96 and 384 indexed reverse transcription primers were synthesized by Sigma Aldrich and shipped at 100 μM in EB Buffer in 96-well plates. Primers had the sequence (5′-TCGTCGGCAGCGTCGGATGCTGAGTGATTGCTTGTGACGCCTTCNNNNNNNNN XXXXXXXXXXXVTTTTTTTTTTTTTTTTTTTVN-3′), where N indicates a random base, the underlined bases are known for a given primer, and X is an 11-base-long primer-specific index sequence. 96-well plates with barcoded oligo-dT primers were prepared prior to the experiment and stored at −20° C. (1 μl of 25 μM per well). 10,000 permeabilized cells or nuclei (2 μl of a 5,000/μl suspension) were added to the pre-dispensed primers and well assignments were recorded. The plate was incubated for 5 min at 55° C. (to resolve RNA secondary structures), then placed immediately on ice (to prevent their re-formation). Per well, a mix of 3 μl nuclease-free water, 2 μl 5× Superscript IV Buffer, 0.5 μl of 100 mM DTT, 0.5 μl of 10 mM dNTPs (Invitrogen cat. no. 18427-088), 0.5 μl of RNaseOUT RNase inhibitor (40 U/ml, Invitrogen cat. no. 10777019), and 0.5 μl of Superscript IV Reverse Transcriptase (200 U/ml, Thermo Fisher Scientific cat. no. 18090200) was added. The reverse transcription was incubated as follows: (heated lid set to 60° C.), 4° C. for 2 min, 10° C. for 2 min, 20° C. for 2 min, 30° C. for 2 min, 40° C. for 2 min, 50° C. for 2 min, 55° C. for 15 min, storage at 4° C.

Second Strand Synthesis and Cell/Nuclei Recovery: For the second strand synthesis, a mix of 1.33 μl Second Strand Synthesis Reaction Buffer and 0.67 μl Second Strand Synthesis Enzyme Mix (NEB cat. no. E6111L) was added per well, followed by 2 hours of incubation at 16° C. Processed nuclei were recovered from the plates and pooled in one 15 ml tube per plate. Wells were washed with 1×PBS-1% BSA, which was transferred to the same tube for maximum recovery. The volume was topped up to 10 ml with 1×PBS-1% BSA, and nuclei were collected (500 rcf, 5 min, 4° C.). We used two additional wash steps with 1×PBS-1% BSA to remove cellular debris. The resulting pellet was resuspended in 1.5 ml of 1× Nuclei Buffer (10× Genomics cat. no. 2000153), transferred to a 1.5 ml tube and centrifuged (500 rcf, 5 min, 4° C.). The supernatant was removed completely, and the tube was centrifuged briefly (500 rcf, 30 s, 4° C.) to collect the remaining liquid at the bottom of the tube. Typically, this resulted in less than 10 μl of a highly concentrated suspension, which was diluted 1:50 and counted in a Fuchs Rosenthal counting chamber (Incyto cat. no. DHC-F01). Tagmentation: For the tagmentation, processed nuclei were combined with 1× Nuclei Buffer for a total volume of 5 μl, and mixed with 7 μl of ATAC Buffer (10× Genomics cat. no. 2000122) and 6 μl of custom i7-only transposome (prepared as described below). Double-stranded cDNA inside the processed nuclei was tagmented at 37° C. for 1 hour, followed by storage at 4° C.

Linear barcoding: Unused channels in the Chromium Chip E (10× Genomics cat. no. 2000121) were filled with 75 μl (inlet 1), 40 μl (inlet 2) or 240 μl (inlet 3) of 50% glycerol solution (Sigma cat. no. G5516-100ML). Right before loading the chip, a mix of 61.5 μl Barcoding Reagent, 1.5 μl Reducing Agent B and 2.0 μl of Barcoding Enzyme (all from 10× Genomics cat. no. 1000110) was added per tagmentation reaction. The microfluidic chip was loaded with 75 μl of tagmented nuclei in barcoding mix (inlet 1), 40 μl of Single Cell ATAC Gel Beads (inlet 2, 10× Genomics cat. no. 2000132) and 240 μl of Partitioning Oil (inlet 3, 10× Genomics cat. no. 220088) and run on the 10× Genomics Chromium controller. The linear barcoding reaction was incubated as follows: (heated lid set to 105° C., volume set to 125 μl), 72° C. for 5 min, 98° C. for 30 s, 12× (98° C. for 10 s, 59° C. for 30 s, 72° C. for 1 min), storage at 15° C. The emulsion was broken by addition of 125 μl of Recovery Agent (10× Genomics cat. no. 220016) and 125 μl of the pink oil phase were removed by pipetting. The remaining sample was mixed with 200 μl of Dynabead Cleanup Master Mix (per reaction: 182 μl Cleanup Buffer (10× Genomics cat. no. 2000088), 8 μl Dynabeads MyOne Silane (Thermo Fisher Scientific cat. no. 37002D), 5 μl Reducing Agent B (10× Genomics cat. no. 2000087), 5 μl of nuclease-free water). After 10 min of incubation at room temperature, samples were washed twice with 200 μl of freshly prepared 80% ethanol (Merck cat. no. 603-002-00-5) and eluted in 40.5 μl of EB Buffer (Qiagen cat. no. 19086) containing 0.1% Tween (Sigma cat. no. P7949-500ML) and 1% v/v Reducing Agent B. Bead clumps were sheared with a 10 μl pipette or needle. 40 μl of the sample were transferred to a fresh tube strip and subjected to a 1.2× cleanup with SPRIselect beads (Beckman Coulter cat. no. B23318), eluting in 40.5 μl of EB Buffer.

Enrichment PCR: Each sample was enriched in eight separate PCR reactions containing 50 μl of NEBNext High Fidelity 2× Master Mix (NEB cat. no. M0541S), 5 μl of primer 06-11_Partial-P5 (10 μM, 5′-AATGATACGGCGACCACCGAGA-3′), 1 μl of 100×SYBR Green in DMSO (Life Technologies cat. no. S7563), 34 μl of water, 5 μl of indexed 06-11_P7-Read2N-00X primer (10 μM, 5′-CAAGCAGAAGACGGCATACGAGAT[indexi7] GTCTCGTGGGCTCGG-3′) and 5 μl of sample from the previous step. Reactions were incubated in a qPCR machine: 98° C. for 45 s, 40× (98° C. for 20 s, 67° C. for 30 s, 72° C. for 30 s followed by the plate read). During the run, the fluorescence signal was monitored and samples were removed from the thermocycler when they reached saturation. To complete unfinished PCR products, the sample was incubated for 2 min at 72° C. in another thermocycler.

Size selection and quality control: PCR reactions were cleaned with a 0.7× standard SPRI cleanup, followed by a double-sided 0.5×/0.7×SPRI cleanup. The library size distribution was checked on a Bioanalyzer HS chip (Agilent cat. no. 5067-4626 and 5067-4627) and the concentration of dsDNA was measured in a Qubit dsDNA HS assay (Thermo Fisher Scientific cat. no. Q32854).

### Example 4-Scifi-RNA-Seq Based on Thermocycling Ligation and Template Switching (Version LIG-TS)

Reverse Transcription: Sets of 96 and 384 indexed reverse transcription primers were synthesized by Sigma Aldrich and shipped at 100 μM in EB Buffer in 96-well plates. Primers had the sequence (5′-[phos]ACACTCTTTCCCTACACGACGCTCTTCCGATCTNNNNNNNNNXXXXXXXXX XXVTTTTTTTTTTTTTTTTTTTTTTTTTTTTTTVN-3′), where N indicates a random base, the underlined bases are known for a given primer, X is an 11-base-long primer-specific index sequence and a 5′ phosphate group allows the ligation of this oligo. 96-well plates with barcoded oligo-dT primers were prepared prior to the experiment and stored at −20° C. (1 μl of 25 μM per well). 10,000 permeabilized cells or nuclei (2 μl of a 5,000/μl suspension) were added to the pre-dispensed primers and well assignments were recorded. The plate was incubated for 5 min at 55° C. (to resolve RNA secondary structures), then placed immediately on ice (to prevent their re-formation). Per well, a mix of 3 μl nuclease-free water, 2 μl 5× Reverse Transcription Buffer, 0.5 μl of 100 mM DTT, 0.5 μl of 10 mM dNTPs (Invitrogen cat. no. 18427-088), 0.5 μl of RNaseOUT RNase inhibitor (40 U/ml, Invitrogen cat. no. 10777019), and 0.5 μl of Maxima H Minus Reverse Transcriptase (200 U/ml, Thermo Fisher Scientific cat. no. EP0753) was added. The reverse transcription was incubated as follows: (heated lid set to 60° C.), 50° C. for 10 min, 3 Cycles of {8° C. for 12 sec, 15° C. for 45 sec, 20° C. for 45 sec, 30° C. for 30 sec, 42° C. for 2 min, 50° C. for 3 min}, 50° C. for 5 min, store at 4° C.

Cell/Nuclei recovery and pooling: Processed cells/nuclei were recovered from the plates and pooled in one 15 ml tube per plate. Wells were washed with 1×PBS-1% BSA, which was transferred to the same tube for maximum recovery. The volume was topped up to 15 ml with 1×PBS-1% BSA, and nuclei were collected (500 rcf, 5 min, 4° C.). The resulting pellet was resuspended in 1.0 ml of 1× HiFi Taq DNA Ligase Buffer (NEB #M0647S) or 1× Ampligase Reaction Buffer (Lucigen #A0102K), filtered through a cell strainer (40 μm or 70 μm depending on the cell/nuclei size) into a 1.5 ml tube and centrifuged (500 rcf, 5 min, 4° C.). The supernatant was removed completely, and the tube was centrifuged briefly (500 rcf, 30 s, 4° C.) to collect the remaining liquid at the bottom of the tube. Typically, this resulted in less than 10 μl of a highly concentrated suspension, which was diluted 1:50 and counted in a Fuchs Rosenthal counting chamber (Incyto cat. no. DHC-F01). The desired number of cells/nuclei was brought to a volume of 15 μl with 1× HiFi Taq DNA Ligase Buffer (NEB #M0647S) or 1× Ampligase Reaction Buffer (Lucigen #A0102K).

Microfluidic thermoligation barcoding: Unused channels in the Chromium Chip E (10× Genomics cat. no. 2000121) were filled with 75 μl (inlet 1), 40 μl (inlet 2) or 240 μl (inlet 3) of 50% glycerol solution (Sigma cat. no. G5516-100ML). Right before loading the chip, a mix of 47.4 μl nuclease-free water, 11.5 μl of either HiFi Taq DNA Ligase Buffer (10×, NEB #M0647S) or Ampligase Reaction Buffer (10×, Lucigen #A0102K), 2.3 μl of either HiFi Taq DNA Ligase (NEB #M0647S) or Ampligase (Lucigen #A0102K), 1.5 μl of Reducing Agent B (10× Genomics cat. no. 2000087) and 2.3 μl of Bridge Oligo (100 HM, 5′-CGTCGTGTAGGGAAAGAGTGTGACGCTGCCGACGA[ddC]-3′) was added per sample. The microfluidic chip was loaded with 75 μl of cells/nuclei in thermoligation mix (inlet 1), 40 μl of Single Cell ATAC Gel Beads (inlet 2, 10× Genomics cat. no. 2000132) and 240 μl of Partitioning Oil (inlet 3, 10× Genomics cat. no. 220088) and run on the 10× Genomics Chromium controller. The thermoligation barcoding reaction was incubated as follows: (heated lid set to 105° C., volume set to 100 μl), 12× (98° C. for 30 s, 59° C. for 2 min), storage at 15° C. The emulsion was broken by addition of 125 μl of Recovery Agent (10× Genomics cat. no. 220016) and 125 μl of the pink oil phase were removed by pipetting. The remaining sample was mixed with 200 μl of Dynabead Cleanup Master Mix (per reaction: 182 μl Cleanup Buffer (10× Genomics cat. no. 2000088), 8 μl Dynabeads MyOne Silane (Thermo Fisher Scientific cat. no. 37002D), 5 μl Reducing Agent B (10× Genomics cat. no. 2000087), 5 μl of nuclease-free water). After 10 min of incubation at room temperature, samples were washed twice with 200 μl of freshly prepared 80% ethanol (Merck cat. no. 603-002-00-5) and eluted in 40.5 μl of EB Buffer (Qiagen cat. no. 19086) containing 0.1% Tween (Sigma cat. no. P7949-500ML) and 1% v/v Reducing Agent B. Bead clumps were sheared with a 10 μl pipette or needle. 40 μl of the sample were transferred to a fresh tube strip and subjected to a 1.0×cleanup with SPRIselect beads (Beckman Coulter cat. no. B23318), eluting in 22 μl of EB Buffer.

Template switching: 20 μl of sample from the previous step were mixed with 10 μl of 5× Reverse Transcription Buffer, 10 μl of Ficoll PM-400 (20%, Sigma #F5415-50ML), 5 μl of 10 mM dNTPs (Invitrogen cat. no. 18427-088), 1.25 μl of Recombinant Ribonuclease Inhibitor (Takara #2313A), 1.25 μl of Template Switching Oligo (100 HM, 5′-AAGCAGTGGTATCAACGCAGAGTGAATrGrGrG-3′, where r indicates RNA bases) and 2.5 μl of Maxima H Minus Reverse Transcriptase (200 U/ml, Thermo Fisher Scientific cat. no. EP0753). The template switching reaction was incubated for 30 min at 25° C., 90 min at 42° C., storage at 4° C. and cleaned with a 1.0×SPRI cleanup, eluting in 17 μl of EB buffer.

cDNA enrichment: 15 μl of the above sample were mixed with 33 μl of nuclease-free water, 50 μl of NEBNext High Fidelity 2× Master Mix (NEB #M0541S), 0.5 μl of Partial P5 primer (100 μM, 5′-AATGATACGGCGACCACCGAGA-3′), 0.5 μl of TSO Enrichment Primer (100 μM, 5′-AAGCAGTGGTATCAACGCAGAGT-3′) and 1 μl of SYBR Green (100× in DMSO). cDNA was amplified in a thermocycler: 98° C. for 30 sec, Cycle until fluorescent signal >2000 RFU {98° C. for 20 sec, 65° C. for 30 sec, 72 C for 3 min}, 72° C. for 5 min in another thermocycler, storage at 4° C. cDNA was cleaned by one 0.8×SPRI cleanup followed by a 0.6×SPRI cleanup, quantified with a Qubit HS assay (ThermoFisher Scientific #Q32854) and 1.5 ng were checked on a Bioanalyzer High-Sensitivity DNA chip (Agilent #5067-4626 and #5067-4627).

Library preparation: cDNA can be converted into NGS-ready libraries by various established methods: (i) tagmentation of double-stranded cDNA with a commercially available (e.g. Illumina Nextera) or custom-made Tn5 transposase (instructions on how to prepare the transposome are included below) followed by PCR enrichment. (ii) fragmentation of double-stranded cDNA by mechanical (e.g. sonication) or enzymatic (e.g. NEB dsDNA fragmentase) means followed by end repair, A-tailing, adapter ligation and PCR enrichment. (iii) linear extension by random priming with a high-processivity polymerase (e.g. Klenow fragment) followed by PCR enrichment.

### Example 5-Scifi-RNA-Seq Based on Linear Extension and Random Priming (EXT-RP)

Random priming (RP) provides an alternative means to introduce a defined sequence at the end of the library fragment distal to the sequence captured during the reverse transcription (e.g. the poly-A tail). It is compatible with version TN5 (where it replaces the tagmentation step) and version LIG (where it replaces the template switching step). Reverse transcription, second strand synthesis and cell/nuclei recovery and counting were performed as described above for version EXT-TN5 (Example 3). However, the tagmentation is no longer required. Instead, processed cells/nuclei in a total volume of 11 μl 1× Nuclei Buffer were mixed with 7 μl of ATAC Buffer (10× Genomics cat. no. 2000122), 61.5 μl Barcoding Reagent, 1.5 μl Reducing Agent B and 2.0 μl of Barcoding Enzyme (all from 10× Genomics cat. no. 1000110) and the microfluidic chip was loaded and run as described previously. The sample was cleaned by silane and SPRI bead cleanups as described above for version EXT-TN5, eluting in a volume of 43 μl nuclease-free water. 41.75 μl of the cleaned sample were mixed with 5 μl of Blue Buffer (10×, Enzymatics #P7010-HC-L), 1.25 μl 10 mM dNTPs (Invitrogen cat. no. 18427-088) and 1 μl of Random Primer (100 μM, 5′-[Btn]GTCTCGTGGGCTCGGAGATGTGTATAAGAGACAGNNNN, where the underlined part corresponds to a stretch of random bases ideally four to eight bases in length and the biotin modification is optional). The sample was then denatured for 5 min at 95° C., and immediately cooled on ice to prevent the re-formation of secondary structures and allow the annealing of the random primer. Next, 1 μl of Klenow Exo-Polymerase (50 U/μl, Enzymatics #P7010-HC-L) was added, the reaction was mixed by pipetting and incubated in a thermocycler: 4° C. for 15 min, then ramp to 37° C. at 1° C./min, 37° C. for 1 hour, then 70° C. for 10 min (enzyme inactivation), storage at 4° C. Excess random primer was removed by addition of 2.5 μl Exonuclease I (20 U/μl, NEB #M0293S) and 1.25 μl of rSAP (1 U/μl, NEB #M0371S) followed by incubation for 1 hour at 37° C. and heat inactivation for 20 min at 80° C., then store at 4° C. After performing a 0.8×SPRI cleanup or a Streptavidin-Bead cleanup, the library was enriched by PCR as described above for version EXT-TN5.

### Example 6: Scifi-RNA-Seq Based on Thermocycling Ligation and Random Priming (Version LIG-RP)

Reverse transcription, cell/nuclei recovery and counting, thermoligation barcoding on the microfluidic device and the silane cleanup are performed as described above for version LIG (Example 4). At the end of the SPRI cleanup, the sample is eluted in 43 μl of nuclease-free water. Random priming replaces the Template Switching step and is performed as follows. 41.75 μl of the cleaned sample are mixed with 5 μl of Blue Buffer (10×, Enzymatics #P7010-HC-L), 1.25 μl 10 mM dNTPs (Invitrogen cat. no. 18427-088) and 1 μl of Random of Primer (100 μM, 5′-[Btn]GTCTCGTGGGCTCGGAGATGTGTATAAGAGACAGNNNN, where the underlined part corresponds to a stretch of random bases ideally four to eight bases in length and the biotin modification is optional). The sample is then denatured for 5 min at 95° C., and immediately cooled on ice to prevent the re-formation of secondary structures and allow the annealing of the random primer. Next, 1 μl of Klenow Exo-Polymerase (50 U/μl, Enzymatics #P7010-HC-L) are added, the reaction is mixed by pipetting and incubated in a thermocycler: 4° C. for 15 min, then ramp to 37° C. at 1° C./min, 37° C. for 1 hour, then 70° C. for 10 min (enzyme inactivation), storage at 4° C. Excess random primer is removed by addition of 2.5 μl Exonuclease I (20 U/μl, NEB #M0293S) and 1.25 μl of rSAP (1 U/μl, NEB #M0371S) followed by incubation for 1 hour at 37° C. and heat inactivation for 20 min at 80° C., then store at 4° C. After performing a 0.8×SPRI cleanup or a Streptavidin-Bead cleanup, the library is enriched by PCR as described above for version EXT-TN5.

### Example 7-Scifi-RNA-Seq Based on Linear Extension and Template Switching (EXT-TS)

Template Switching (TS) provides an alternative means to introduce a defined sequence at the end of the library fragment distal to the sequence captured during the reverse transcription (e.g. the poly-A tail). TS is already used in version LIG-TS, but is also compatible with version EXT-TN5, as described below. Reverse transcription is performed with Maxima H Minus Reverse Transcriptase or an alternative reverse transcriptase that adds untemplated C bases to the cDNA upon reaching the transcript end. Reverse transcription primers have the sequence (5′-TCGTCGGCAGCGTCGGATGCTGAGTGATTGCTTGTGACGCCTTCNNNNNNNNN XXXXXXXXXXXVTTTTTTTTTTTTTTTTTTTTTTTVN-3′), where N indicates a random base, the underlined bases are known for a given primer, and X is an 11-base-long primer-specific index sequence. 96-well plates with barcoded oligo-dT primers are prepared prior to the experiment and stored at −20° C. (1 μl of 25 μM per well). 10,000 permeabilized cells or nuclei (2 μl of a 5,000/μl suspension) are added to the pre-dispensed primers and well assignments are recorded. The plate is incubated for 5 min at 55° C. (to resolve RNA secondary structures), then placed immediately on ice (to prevent their re-formation).

Per well, a mix of 1 μl 5× Reverse Transcription Buffer, 1 μl of Ficoll PM-400 (20%, Sigma #F5415-50ML), 0.5 μl of 10 mM dNTPs (Invitrogen cat. no. 18427-088), 0.125 μl of Recombinant Ribonuclease Inhibitor (Takara #2313A), 0.125 μl of Template Switching Oligo (100 μM, 5′-AAGCAGTGGTATCAACGCAGAGTGAATrGrGrG-3′, where r indicates RNA bases) and 0.25 μl of Maxima H Minus Reverse Transcriptase (200 U/ml, Thermo Fisher Scientific cat. no. EP0753) is added. The combined reverse transcription and template switching reaction is incubated as follows: (heated lid set to 60° C.), 25° C. for 30 min, 42° C. for 90 min, storage at 4° C. Cell/nuclei recovery and counting are performed as described above for version EXT-TN5. However, the tagmentation is no longer required. Instead, processed cells/nuclei in a total volume of 9.7 μl 1× Nuclei Buffer are mixed with 7 μl of ATAC Buffer (10× Genomics cat. no. 2000122), 61.5 μl Barcoding Reagent, 1.5 μl Reducing Agent B, 2.0 μl of Barcoding Enzyme (all from 10× Genomics cat. no. 1000110) and 1.3 μl of TSO Enrichment Primer (100 μM, 5′-AAGCAGTGGTATCAACGCAGAGT-3′). The microfluidic chip is loaded and run and the droplet emulsion is incubated as described previously. The sample is cleaned by silane and SPRI bead cleanups as described above for version EXT-TN5. The cDNA is amplified and libraries are prepared as described above for version LIG-TS.

### Example 8-Assembly of the Custom i7-Only Transposome

Oligonucleotides Tn5-top_ME (5′-[Phos]CTGTCTCTTATACACATCT-3′) and Tn5-bottom_Read2N (5′-GTCTCGTGGGCTCGGAGATGTGTATAAGAGACAG-3′) were synthesized by Sigma Aldrich and reconstituted in EB buffer (Qiagen cat. no. 19086) at 100 μM. 22.5 μl of each oligonucleotide and 5 μl of 10× Oligonucleotide Annealing Buffer (10 mM Tris-HCl (Sigma cat. no. T2944-100ML), 50 mM NaCl (Sigma cat. no. S5150-1L), 1 mM EDTA (Invitrogen cat. no. AM9260G)) were mixed and annealed in a thermocycler: 95° C. for 3 min, 70° C. for 3 min, ramp to 25° C. at 2° C. per minute. The annealing reaction was then diluted by addition of 180 μl of water. At this point, the diluted oligonucleotide cassette can be aliquoted and frozen for future transposome assemblies. To load the Tn5 transposase, we mixed 20 μl of diluted oligonucleotide cassette from the previous step with 20 μl of 100% glycerol (Sigma cat. no. G5516-100ML) and 10 μl of EZ-Tn5 Transposase (Lucigen cat. no. TNP92110), and incubated for 30 min at 25° C. in a thermocycler. The resulting 50 μl of assembled transposome are sufficient for eight scifi-RNA-seq reactions with the EXT-TN5 protocol (6 μl per reaction) or over 200 library preparations for scifi-RNA-seq implementations with cDNA enrichment. The transposome can be stored at −20° C. for at least one month.

### Example 9-Activity Check for the Custom i7-Only Transposome by qPCR

Tagmented DNA flanked by two Illumina i7 adapters is suppressed in PCR reactions due to competition between intramolecular annealing and primer binding. The custom i7-only transposome is therefore tested in a negative qPCR assay as described previously (Rykalina et al., 2017). Briefly, a defined PCR product is subjected to one tagmentation reaction and one no-enzyme control reaction. Both samples are then re-amplified with the same primers in a qPCR reaction. Since the tagmentation fragments the PCR product, the corresponding reaction should yield higher Ct values. The tagmentation efficiency can then be calculated from the shift of Ct values:
Tagmentation efficiency [%]=100/[2 \hat{ }(average Ct tagmentation−average Ct no-enzyme control)].

Generation of the PCR product: Oligonucleotides pUC19-FWD (5′-AAGTGCCACCTGACGTCTAAG-3′) and pUC19-REV (5′-CAACAATTAATAGACTGGATGGAGGCGG-3′) were synthesized by Sigma Aldrich and reconstituted in EB buffer (Qiagen cat. no. 19086) at 100 μM. Next, a 1,961 bp PCR product was generated by mixing 128.7 μl of water, 33 μl of 50 pg/μl pUC19 plasmid (NEB cat. no. N3041S), 1.65 μl each of primers pUC19-FWD and pUC19-REV (100 μM) combined with 165 μl of 2× Q5 HotStart High-Fidelity Master Mix (NEB cat. no. M0494L). The resulting 6.6× master mix was distributed into a tube strip (six reactions of 50 μl) and amplified in a thermocycler: 98° C. for 30 s; 31× (98° C. for 10 s, 68° C. for 30 s, 72° C. for 1 min), 72° C. for 2 min, storage at 12° C. To each 50 μl PCR reaction, we added 6.25 μl of 10× CutSmart Buffer and 6.25 μl of Dpnl (NEB cat. no. R0176L) and incubated at 37° C. for 1 hour to digest the PCR template plasmid. The six PCR reactions were pooled and cleaned with the QiaQuick PCR Purification Kit (Qiagen cat. no. 28106) using two columns and eluting with 30 μl of EB buffer per column. Eluates were pooled, and the purity of the PCR fragment was checked on a 1% agarose gel containing ethidium bromide. We then measured the concentration of dsDNA with a Qubit HS assay (Thermo Fisher Scientific cat. no. Q32854), and diluted the PCR product to 25 ng/μl with EB buffer.

Tagmentation: Tagmentation reactions were set up by mixing 2 μl of 25 ng/μl pUC19 PCR product from the previous step, 7 μl of ATAC Buffer (10× Genomics cat. no. 2000122), and either 6 μl of custom i7-only transposome (tagmentation reaction) or 6 μl of water (no-enzyme control reaction). After 60 min of incubation at 37° C., the Tn5 enzyme was stripped from the DNA by addition of 1.75 μl of 1% SDS solution (Sigma cat. no. 71736-100ML) followed by incubation at 70° C. for 10 min. The two reactions were diluted 1/100 with EB buffer, and qPCR reactions were set up in triplicates: 2 μl of 1/100-diluted reaction, 10 μl of 2× GoTaq qPCR Master Mix (Promega cat. no. A600A), 0.1 μl each of 100 μM pUC19-FWD and pUC19-REV primers and 7.8 μl of water. qPCR reactions were incubated as follows: 95° C. for 2 min, 40× (95° C. for 30 s, 68° C. for 30 s, 72° C. for 2 min and plate read).

### Example 10-Next-Generation Sequencing

Resulting scifi-RNA-seq libraries were sequenced on the Illumina NextSeq 500 platform, using High Output v2.5 reagents (75 Cycles, Illumina cat. no. 20024906). We used custom sequencing primers 18-12_scifi_SEQ_inDrop_read1 (5′-GGATGCTGAGTGATTGCTTGTGACGCC*T*T*C, where * denotes phosphorothioate bonds) for Read1 and 18-12_scifi_SEQ_inDrop_index2 (5′-GCATCCGACGCTGCCGA*C*G*A-3′) for Index2. The machine was set to read lengths of 21 bases (Read1), 47 bases (Read2), 8 bases (Index1, i7) and 16 bases (Index2, i5).

Large single-cell libraries were sequenced on the Illumina NovaSeq 6000 platform, using NovaSeq 6000 SP (100 Cycles, Illumina cat. no. 20027464) or S2 (100 Cycles, Illumina cat. no. 20012862) reagents. Custom sequencing primer 18-12_scifi_SEQ_inDrop_read1 (5′-GGATGCTGAGTGATTGCTTGTGACGCC*T*T*C, where * denotes phosphorothioate bonds) was applied for Read1. Due to a different sequencing chemistry, Index2 can be read with standard NovaSeq primers. The sequencer was set to a read structure of 21 bases (Read1), 55 bases (Read2), 8 bases (Index1, i7) and 16 bases (Index2, i5).

In some implementations of scifi-RNA-seq, a primer binding site compatible with standard Illumina sequencing primers was used, so that custom primers were no longer required.

### Example 11-Scifi-RNA-Seq on a 1:1 Mixture of Human and Mouse Cells

Cell culture: Human Jurkat-Cas9-TCRlib cells were cultured in RPMI medium (Gibco #21875-034) containing 10% FCS (Sigma) and penicillin-streptomycin and were continuously selected with 25 μg/ml blasticidin (Invivogen #ant-bl-5) and 2 μg/ml puromycin (Fisher Scientific #A1113803). Mouse 3T3 cells were cultured in DMEM medium (Gibco #10569010) containing 10% FCS (Sigma) and penicillin-streptomycin.

Single-cell RNA-seq: A nuclei suspension from human Jurkat-Cas9-TCRlib cells and mouse 3T3 cells was freshly prepared, as described in Example 1.2, supra. To evaluate the performance of scifi-RNA-seq as a function of droplet overloading, 15,300, 383,000, or 765,000 pre-indexed nuclei were loaded into a single channel of the Chromium system. Both the number of single-cell transcriptomes and the average number of nuclei inside each droplet scaled linearly with the loading amount (FIG. 19). In addition, this dataset, which was based on a 1:1 mixture of human and mouse cell lines, allowed us to validate our pre-indexing strategy for the correct assignment of transcripts to single cells. To that end, we compared the number of human-mouse cell doublets based on the microfluidic (round2) barcode only with the number of such doublets based on the combination of pre-indexing (round1) and microfluidic (round2) barcodes (FIG. 20). As expected for a loading rate of 765,000 nuclei per channel, almost all droplets contained both human and mouse cells (FIG. 20, left panel), but the vast majority of these doublets could be resolved when considering both the round1 and round2 barcode (FIG. 20, right panel). As expected, the striking effect of pre-indexing was seen only when the droplet generator was overloaded, while the microfluidic round2 barcode alone was sufficient for minimizing cell doublets at a standard loading rate of 15,300 nuclei per channel (FIG. 33).

Finally, the dataset allowed to conclusively resolve a third feasibility concern for scifi-RNA-seq-whether the reagents in each droplet would be sufficient for effective barcoding of the transcriptomes from multiple nuclei. When plotting UMI counts and fractions of unique reads per cell against the number of nuclei per droplet (FIG. 21), no trend towards lower transcriptome complexity was observed in droplets containing up to 15 individual nuclei, strongly suggesting that reagents for droplet-based indexing are not a limiting factor in scifi-RNA-seq.

### Example 12-Scifi-RNA-Seq on a Mixture of Four Human Cell Lines

Cell culture: Jurkat-Cas9-TCRlib, K562 and NALM-6 cell lines were cultured in RPMI medium (Gibco #21875-034) containing 10% FCS (Sigma) and penicillin-streptomycin. Jurkat-Cas9-TCRlib cells were continuously selected with 25 μg/ml blasticidin (Invivogen #ant-bl-5) and 2 μg/ml puromycin (Fisher Scientific #A1113803). HEK293T cells were cultured in DMEM medium (Gibco #10569010) containing 10% FCS (Sigma) and penicillin-streptomycin.

Single-cell RNA-seq: A nuclei suspension from four human cell lines with unique characteristics (Jurkat, K562, NALM-6, HEK293T) was freshly prepared, as described in Example 1.2, supra. Next, these nuclei were subjected to scifi-RNA-seq as described in Example 4, supra, according to the protocol based on thermocycling ligation and template switching (LIG-TS). During the reverse transcription step on a 384-well plate, each cell line was assigned a specific set of pre-indexing (round1) barcodes. After the pre-indexing samples were pooled and 383,000 nuclei were loaded into a single microfluidic channel of the Chromium system. 151,788 single-cell transcriptomes passed quality control (FIGS. 22-23, 35-36), which constitutes a 15-fold increase over the output of the standard Chromium protocol. This experiment also allowed to demonstrate the method's inherent support for multiplexing of up to 384 distinct samples in a single experiment.

### Example 13-Scifi-RNA-Seq on Primary Human T Cells

Isolation of primary human T cells: Peripheral blood from healthy donors was obtained from as blood packs with buffered sodium citrate as anti-coagulant. For each donor, we prepared T cells from 3×15 ml of peripheral blood, according to the following protocol. 15 ml of peripheral blood were mixed with 750 μl of RosetteSep Human T Cell Enrichment Cock-tail (Stemcell #15061). After 10 min of incubation at room temperature, the sample was diluted by addition of 15 ml 1×PBS (Gibco #14190-094) containing 2% v/v FCS (Sigma). SepMate tubes (Stemcell #86450) were loaded with 15 ml of Lymphoprep density gradient medium (Stemcell #07851) and the blood sample was poured on top. After centrifugation (1,200 rcf, 10 min, room temperature, brake set to 9), the supernatant was transferred to a fresh 50 ml tube, topped up to 50 ml with 1×PBS containing 2% FCS, and centrifuged (1200 rcf, 10 min, room temperature, brake set to 3). After one additional wash with 50 ml of 1×PBS containing 2% FCS (1200 rcf, 10 min, room temperature, brake set to 3), T cells were resuspended in 10 ml of 1×PBS containing 2% FCS, filtered through a 40 μM cell strainer, and counted using a CASY device (Schärfe Systems). For accurate cell counting, it was important to exclude contaminating erythrocytes, which will be lysed during the subsequent nuclei preparation.

Anti-CD3/CD28 stimulation of human T cells: Freshly isolated primary human T cells were resuspended at a density of 1 million cells per ml in Human T Cell Medium (OpTmizer medium (Thermo Fisher #A1048501) containing 1/38.5 volumes of OpTmizer supplement, 1× GlutaMax (Thermo Fisher #35050061), 1× Penicillin/Streptomycin (Thermo Fisher #15140122), 2% heat-inactivated human AB serum (Fisher Scientific #MT35060CI), 10 ng/ml of recombinant human IL-2 (PeproTech #200-02)). The culture was split into two flasks, and one was treated with Human T-Activator CD3/CD28 Dynabeads (25 μl beads per 1 million cells, Thermo Fisher #11131D). After 16 hours, we prepared formaldehyde-fixed nuclei and snap-froze the nuclei suspension as described herein.

Flow cytometry analysis of T cell populations: A total of 1 million primary human T cells were washed twice with 1×PBS containing 0.1% BSA and 5 mM EDTA (PBS-BSA-EDTA). Single-cell suspension was incubated with anti-CD16/CD32 (clone 93, 1:200, Biolegend #101301) to prevent nonspecific binding and stained with combinations of antibodies against CD4 (PE-TxRed, clone OKT4, 1:200, Biolegend #317448), CD8 (APC-Cy7, clone SK1, 1:150, Biolegend #344746), CD25 (PE-Cy7, clone BC96, 1:100, Biolegend #302612), CD45RA (PerCp-Cy5.5, clone HI100, 1:100, Biolegend #304122), CD45RO (AF700, clone UCHL1, 1:100, Biolegend #304218), CD69 (AF488, clone FN50, 1:100, Biolegend #310916), CD127 (APC, clone A019D5, 1:100, Biolegend #351342), CD197 (CCR7, PE, clone G043H7, 1:100, Biolegend #353204), and DAPI viability dye (Biolegend #422801) for 30 min at 4° C. After two washes with PBS-BSA-EDTA, cells were acquired with an LSRFortessa Cell Analyzer (BD). CD4+ and CD8+ T cells were subdivided into naive T cells (CD45RA+CCR7+), effector memory T cells (CD45RA-CCR7−), central memory T cells (CD45RA-CCR7+) and TEMRA cells (CD45RA+CCR7−). T cell receptor-mediated activation of CD4+ and CD8+ T cells was assessed based on CD25 and CD69 expression.

Single-cell RNA-seq: scifi-RNA-seq was performed as described in Example 4, following the protocol based on thermocycling ligation and template switching (LIG-TS). During the reverse transcription step on a 384-well plate, donor identity and TCR stimulation status were barcoded with a set of unique round1 pre-indices. After the pre-indexing samples were pooled and 765,000 nuclei were loaded into a single microfluidic channel of the Chromium system. Results are shown in FIGS. 24-25,37-38.

### Example 14-Comparison to Existing Combinatorial Indexing Protocols

In this experiment the performance of the methods of the invention was compared to existing multi-round combinatorial indexing technologies. Publicly available data was obtained for sci-RNA-seq v1 (Cao, Packer et al., 2017), SPLIT-seq (Rosenberg, Roco et al, 2018), sci-RNA-seq v3 (Cao, Spielmann et al., 2019) and sci-Plex (Srivatsan, McFaline-Figueroa, Ramani et al., 2020). Using mouse 3T3 cells as a common point of reference, it was demonstrated that the library quality of scifi-RNA-seq was consistently superior to sci-RNA-seq v1, sci-RNA-seq v3 and sci-Plex (FIG. 44a-f), and a greatly reduced percentage of doublet cells in human/mouse species mixing experiments was observed (FIG. 44g). The data quality of scifi-RNA-seq was more reproducible than for SPLIT-seq, where highly variable results for two replicate samples were obtained (FIG. 44a-f).

In addition, the library design and sequencing read structure was compared between the methods, in order to assess their cost-effectiveness. Because scifi-RNA-seq does not read uninformative ligation overhangs, all sequencing cycles spent on cell barcodes are informative, in contrast to sci-RNA-seq v1 (58% informative), sci-RNA-seq v3 and sci-Plex (87% informative), and SPLIT-seq (33% informative). As a result, scifi-RNA-seq greatly reduces the bottleneck of sequencing cost for ultra-high throughput single-cell RNA-seq. (FIG. 44h-i). In summary, it was found that scifi-RNA-seq resulted in superior data quality and reproducibility, greatly reduces the experimental workload, and can be performed faster than existing methods.

### Example 15-Comparison to the 10× Genomics Chromium Platform

For a comparison to microfluidic single-cell RNA-seq, scifi-RNA-seq was benchmarked against the widely used 10× Genomics technology, using the latest v3 chemistry. In a series of new wet-lab experiments, test samples were split and processed side-by-side with both assays, loading the same number of 7,500 nuclei/cells per microfluidic channel and comparing the results between permeabilized nuclei, methanol-fixed cells, and intact cells. An equal mixture of four human cell lines with variable transcript content was used (K562, HEK293T, Jurkat, NALM-6) as well as a cross-species mixture of human (Jurkat) and mouse (3T3) cells. This setup allowed separation of the effects of permeabilization methods, technology platform, cell type, species and transcript content.

In summary, these experiments showed that: (i) Pre-indexed cells/nuclei in scifi-RNA-seq are recovered at almost the same rate as native cells/nuclei on the 10× Genomics system. Due to the minimal sequencing coverage spent on background this can be compensated for by increased loading concentrations (FIG. 39a). (ii) The wash and filtration steps in scifi-RNA-seq efficiently remove permeabilization artefacts such as free-floating RNA and cell fragments that were common in 10× Genomics data of nuclei and methanol-fixed cells, further demonstrating the advantage of the claimed protocols (FIG. 39a). (iii) Spurious clusters of doublet cells were commonly detected in 10× Genomics data but completely absent from scifi-RNA-seq data, demonstrating the methods vastly larger barcoding capacity. (FIG. 39b and FIG. 43a-c) (iv) The four human cell lines were recovered at equal proportions, indicating that there was little cell-type specific sampling bias, or bias due to transcript content (FIG. 39c). (v) Gene expression profiles correlated by the cell line, irrespective of the technology (scifi-RNA-seq vs 10× Genomics) and sample preparation method (nuclei, methanol-fixed cells, intact cells). (v) While, by design, no combinatorial indexing method is expected to reach the library complexity of direct single-cell RNA-seq using the latest 10× Genomics v3 chemistry, the data quality of scifi-RNA-seq does not fall behind, while providing greatly increased cell throughput (at least 15× more cells per run).

### Example 16-Compatibility with Chromium Single Cell ATAC v.1.1 (NextGEM) Design

It was shown that droplet overloading according to the methods of the present invention is compatible with the Chromium Single Cell ATAC v.1.1 (NextGEM) kit (FIG. 41a). All tested loading concentrations resulted in a stable, monodispersed droplet emulsion (FIG. 41b) and the droplet fill rate and number of nuclei per droplet increased in a controlled fashion with the loading concentrations, ranging from 15,000 to 1.53 million nuclei per channel. Design-specific differences of the NextGEM compared to the original chip design were identified, specifically higher numbers of nuclei per droplet, a superior bead loading rate and a greatly reduced number of empty droplets. It was also demonstrated that the droplet diameter is highly similar between platforms and does not change when overloading droplets with nuclei (FIG. 41c-d). Based on the NextGEM-specific data, the nuclei loading into droplets was computationally modeled (FIG. 41e-g), and the droplet fill rate and nuclei loading distributions were visualized (FIG. 41h-i), and the expected percentages of cell doublets for different numbers of round1 pre-indexing barcodes were determined (FIG. 41j). Finally, the methods of the invention were applied in parallel using scATAC v1.0 and v1.1 (NextGEM) reagents and it was demonstrated that the data quality and single cell purity are comparable (FIG. 42a-e). In conclusion, these experiments demonstrated that the methods of the invention are perfectly compatible with the NextGEM chip design, both in terms of droplet overloading and the enzymatic reactions.

### Example 17-Scifi Multiplexing Enables Large-Scale Perturbation Screens at the Single Cell Level

The advantages of the whole transcriptome pre-indexing step in scifi-RNA-seq are two-fold. First, barcoded cells/nuclei can be loaded into the second compartment at a rate of multiple cells/nuclei per compartment, allowing the ultra-high throughput processing of the sample. Secondly, the round1 pre-index can label hundreds to thousands of experimental conditions, thereby enabling large-scale perturbation studies such as drug screens or genetic perturbation screens at the single-cell level.

To demonstrate the multiplexing capabilities of the invention, and the benefits of profiling very high numbers of single-cells for drug development and target discovery, the following experiment was performed. The human Jurkat cell line was transduced with a lentiviral vector to express the Cas9 nuclease. These cells were further modified with a second lentiviral vector expressing 48 distinct CRISPR guide RNAs (gRNAs), targeting 20 genes with 2 gRNAs each plus 8 non-targeting control gRNAs. We allowed 10 days for efficient genome editing under antibiotic selection. Afterwards, the 48 single knockout cell lines were split into two parts, which received stimulation of the T cell receptor with anti-CD3/CD28 antibodies or were left untreated. For the resulting 96 samples methanol-fixed cells were prepared and scifi-RNA-seq according to the described methods of the invention was performed (FIG. 40a). A signature of 300 genes differentially expressed between the stimulated and unstimulated conditions was used to define a T-cell receptor activation score for each gene knockout (FIG. 40c). Using the transcriptome data from this screen, key regulators of the T-cell receptor pathway were identified, such as the kinases ZAP70 and LCK, the adaptor protein LAT, and the phosphatase PTPN11 at both the level of bulk transcriptomes (FIG. 40b-d) and at the single-cell level (FIG. 40e-g).

The above highlights the potential of the methods of the invention for drug discovery and target validation. The methods of the invention derive relevant screening signatures directly from the transcriptome of control cells, so that no prior knowledge about the mechanism of action of a drug is required. This can save valuable time in prioritizing lead candidates and in bringing a drug product to the market. Moreover, the single-cell resolution of the methods of the invention can assess the effect of drug treatments on different cell types in a complex mixture (for instance PBMCs), or on a mixture of cells from distinct donors.

1. A method for sequencing oligonucleotides comprising RNA, the method comprising the steps of:
(a) providing permeabilized cells and/or nuclei comprising a first oligonucleotide comprising RNA;
(b) combining said cells and/or nuclei of (a) with a second oligonucleotide comprising DNA in a first reaction compartment, wherein the second oligonucleotide comprises at least a first sequence at least partially complementary to a sequence of the first oligonucleotide, a second sequence comprising an indexing sequence, and a third sequence comprising a primer binding site, under conditions to allow annealing of the first sequence of the second oligonucleotide to the first oligonucleotide;
(c) reversely transcribing the first oligonucleotide in said cells/nuclei to obtain an elongated second oligonucleotide;
(d) combining said cells and/or nuclei obtained in step (c) with a microbead-bound third oligonucleotide in a second reaction compartment, wherein the third oligonucleotide comprises
(i) a first sequence corresponding to a fourth sequence comprised in the second oligonucleotide used in step (b); or
(ii) a first sequence complementary to a first sequence of a fourth oligonucleotide, wherein the fourth oligonucleotide further comprises a second sequence at least partially complementary to the third sequence of the second oligonucleotide;
wherein for (i), the method further comprises a step of second strand DNA synthesis subsequent to step (c) and prior to step (d) and wherein for (ii) the method further comprises a step of DNA ligation;
and wherein the third oligonucleotide further comprises a second sequence comprising an indexing sequence and a third sequence comprising a primer binding site;
(e) amplifying the DNA oligonucleotides obtained in step (d); and
(f) sequencing of amplified DNA oligonucleotides.

2. The method of claim 1, wherein in step (c) untemplated nucleotides are added to the 3′-end of the second oligonucleotide.

3. The method of claim 2, wherein second strand DNA synthesis comprises the use of primers comprising a sequence complementary to the added untemplated nucleotides.

4. The method of claim 2, wherein a primer comprising RNA nucleotides complementary to the added untemplated nucleotides is added for extension.

5. The method of claim 1, wherein second strand DNA synthesis comprises
(a) introducing nicks in the first oligonucleotide;
(b) extending nicked oligonucleotides; and
(c) ligating extended oligonucleotides.

6. The method of claim 1, further comprising subsequent to or concurrently with second strand DNA synthesis a step of introducing untemplated nucleotides at the 5′-end of the synthesized second strand DNA.

7. The method of claim 6, wherein untemplated nucleotides are introduced using a transposase enzyme.

8. The method of claim 1, wherein the method further comprises a step of linear extension subsequent to DNA ligation, wherein linear extension comprises adding a primer comprising RNA nucleotides and adding a reverse transcriptase enzyme.

9. The method of claim 1, wherein the method further comprises a step of linear extension comprising adding a primer comprising random nucleotides.

10. The method of claim 1, wherein the sequence of the first oligonucleotide bound by the first sequence of the second oligonucleotide is located at the 3′-end of the first oligonucleotide.

11. The method of claim 1, wherein the first sequence of the second oligonucleotide is complementary to the 3′ poly-A tail of the first oligonucleotide.

12. The method of claim 1, wherein the first reaction compartment comprises permeabilized intact cells and/or nuclei.

13. (canceled)

14. The method of claim 1, wherein the second reaction compartment comprises lysed cells and/or nuclei.

15. (canceled)

16. The method of claim 1, wherein the second reaction compartment is a microfluidic droplet or a well on a microtiter plate.

17. The method of claim 16, wherein the second reaction compartment is a microfluidic droplet and the third oligonucleotide is released from the microbead upon formation of the droplets.

18. The method of claim 1, wherein the second oligonucleotide further comprises a unique molecular identifier (UMI).

19. The method of claim 1, wherein the cells and/or nuclei are obtained from in vitro cultures or fresh or frozen samples.

20. The method of claim 1, wherein the cells and/or nuclei are
(a) obtained from existing cell lines, primary cells, blood cells, somatic cells, derived from organoids or xenografts;
(b) CAR-T cells, CAR-NK cells, modified T-cells, B-cells, NK cells, immune cells, or isolated from patients treated with such products; or
(c) pluripotent stem cells (iPS) or embryonic stem cells undergoing natural differentiation or artificially induced reprogramming or transdifferentiation.

21. The method of claim 1, wherein DNA ligation uses a thermostable DNA ligase.

22-24. (canceled)

25. A kit comprising a second oligonucleotide comprising at least a first sequence at least partially complementary to a sequence of a first RNA oligonucleotide, a second sequence comprising an indexing sequence, and a third sequence comprising a primer binding site, optionally together with instructions regarding the use of the second oligonucleotide in the method of claim 1.

26-28. (canceled)


SEQUENCE LISTING (19 sequences)

SEQ ID NO: 1 - DNA, 97 bp, Artificial Sequence
  Reverse transcription primer
  misc_feature (45)..(64): n = a, t, g, or c
  misc_feature (97)..(97): n = a, t, g, or c
tcgtcggcagcgtcggatgctgagtgattgcttgtgacgccttcnnnnnnnnnnnnnnnnnnnnvttttttttttttttttttttttttttttttvn

SEQ ID NO: 2 - DNA, 22 bp, Artificial Sequence
  primer 06-11_Partial-P5
aatgatacggcgaccaccgaga

SEQ ID NO: 3 - DNA, 47 bp, Artificial Sequence
  indexed 06-11_P7-Read2N-00X primer
  misc_feature (25)..(32): n = t, g, a, or c
caagcagaagacggcatacgagatnnnnnnnngtctcgtgggctcgg

SEQ ID NO: 4 - DNA, 86 bp, Artificial Sequence
  Reverse transcription primer
  modified_base (1)..(1): 5′ phosphorylation
  misc_feature (34)..(53): n = a, t, g, or c
  misc_feature (86)..(86): n = a, t, g, or c
acactctttccctacacgacgctcttccgatctnnnnnnnnnnnnnnnnnnnnvttttttttttttttttttttttttttttttvn

SEQ ID NO: 5 - DNA, 36 bp, Artificial Sequence
  Bridge Oligo
  modified_base (36)..(36): dideoxycytidine
cgtcgtgtagggaaagagtgtgacgctgccgacgac

SEQ ID NO: 6 - DNA, 30 bp, Artificial Sequence
  Template Switching Oligo
  misc_feature (28)..(30): n = RNA base g
aagcagtggtatcaacgcagagtgaatnnn

SEQ ID NO: 7 - DNA, 23 bp, Artificial Sequence
  TSO Enrichment Primer
aagcagtggtatcaacgcagagt

SEQ ID NO: 8 - DNA, 38 bp, Artificial Sequence
  Random primer
  modified_base (1)..(1): biotin modification (optional)
  misc_feature (35)..(38): n = a, t, c, or g
gtctcgtgggctcggagatgtgtataagagacagnnnn

SEQ ID NO: 9 - DNA, 19 bp, Artificial Sequence
  Oligonucleotide Tn5-top_ME
  modified_base (1)..(1): 5′ phosphorylation
ctgtctcttatacacatct

SEQ ID NO: 10 - DNA, 34 bp, Artificial Sequence
  Oligonucleotide Tn5-bottom_Read2N
gtctcgtgggctcggagatgtgtataagagacag

SEQ ID NO: 11 - DNA, 21 bp, Artificial Sequence
  Oligonucleotide pUC19-FWD
aagtgccacctgacgtctaag

SEQ ID NO: 12 - DNA, 28 bp, Artificial Sequence
  Oligonucleotide pUC19-REV
caacaattaatagactggatggaggcgg

SEQ ID NO: 13 - DNA, 30 bp, Artificial Sequence
  Sequencing primer 18-12_scifi_SEQ_inDrop_read1
  misc_feature (27)..(27): linkage to the next base is a phosphorothioate bond
  misc_feature (28)..(28): linkage to the next base is a phosphorothioate bond
  misc_feature (29)..(29): linkage to the next base is a phosphorothioate bond
ggatgctgagtgattgcttgtgacgccttc

SEQ ID NO: 14 - DNA, 20 bp, Artificial Sequence
  Sequencing primer 18-12_scifi_SEQ_inDrop_index2
  misc_feature (17)..(17): linkage to the next base is a phosphorothioate bond
  misc_feature (18)..(18): linkage to the next base is a phosphorothioate bond
  misc_feature (19)..(19): linkage to the next base is a phosphorothioate bond
gcatccgacgctgccgacga

SEQ ID NO: 15 - DNA, 23 bp, Artificial Sequence
  hU6 no extension
cttgtggaaaggacgaaacaccg

SEQ ID NO: 16 - DNA, 36 bp, Artificial Sequence
  hU6 shorter
gtctcgtgggctcggtgtggaaaggacgaaacaccg

SEQ ID NO: 17 - DNA, 55 bp, Artificial Sequence
  hU6 short
gtctcgtgggctcggagatgtgtataagagacagtgtggaaaggacgaaacaccg

SEQ ID NO: 18 - DNA, 87 bp, Artificial Sequence
  hU6 full Nextera
caagcagaagacggcatacgagattcgccttagtctcgtgggctcggagatgtgtataagagacagtgtggaaaggacgaaacaccg

SEQ ID NO: 19 - DNA, 27 bp, Artificial Sequence
  gRNA backbone REV1
gtgtctcaagatctagttacgccaagc